SEQUENCE LISTING (52 sequences)

SEQ ID NO: 1 - protein, 8 aa, Artificial Sequence
  Description of Artificial Sequence Synthetic peptide
GFTFEAYA

SEQ ID NO: 2 - protein, 8 aa, Artificial Sequence
  Description of Artificial Sequence Synthetic peptide
INWNSGRI

SEQ ID NO: 3 - protein, 16 aa, Artificial Sequence
  Description of Artificial Sequence Synthetic peptide
AKDIRRFSTGGAEFEY

SEQ ID NO: 4 - protein, 8 aa, Artificial Sequence
  Description of Artificial Sequence Synthetic peptide
SSNIGSNF

SEQ ID NO: 5 - protein, 3 aa, Artificial Sequence
  Description of Artificial Sequence Synthetic peptide
RNN

SEQ ID NO: 6 - protein, 12 aa, Artificial Sequence
  Description of Artificial Sequence Synthetic peptide
AAWDDSLGGHYV

SEQ ID NO: 7 - protein, 142 aa, Artificial Sequence
  Description of Artificial Sequence Synthetic polypeptide
MEFGLSWLFLVAILKGVQCQVQLVESGGGSVQPGRSLRLSCEASGFTFEAYAMHWVRQPPGKGLEWVSSINWNSGRIAYADSVKGRFTISRDNARNSLYLQMNSLRLEDTAFYYCAKDIRRFSTGGAEFEYWGQGTLVTVSS

SEQ ID NO: 8 - protein, 130 aa, Artificial Sequence
  Description of Artificial Sequence Synthetic polypeptide
MAGFPLLLTLLTHCAGSWAQSVLTQPPSASGTPGQRVTISCSGSSSNIGSNFVYWYQQLPGTAPKLLIYRNNQRPSGVPDRFSGSRSGTSASLAISGLRSEDEADYYCAAWDDSLGGHYVFGTGTKVTVL

SEQ ID NO: 9 - DNA, 426 bp, Artificial Sequence
  Description of Artificial Sequence Synthetic polynucleotide
atggagtttgggctgagctggctttttcttgtggctattttaaaaggcgtacagtgccaggtgcagctggtggagtctgggggaggctcggtgcagcctggcaggtccctgagactctcctgtgaagcctctggattcacctttgaggcctatgccatgcactgggtccggcaacctccagggaagggcctggagtgggtctcaagtattaattggaatagtggtcgcatagcctatgcggactctgtgaagggccgattcaccatctccagagacaacgccaggaattccctgtatctgcaaatgaacagtctgagacttgaggacacggccttctattactgtgcaaaagatatacggaggtttagtaccgggggggcggagtttgagtactggggccagggaaccctggtcaccgtctcctca

SEQ ID NO: 10 - DNA, 390 bp, Artificial Sequence
  Description of Artificial Sequence Synthetic polynucleotide
atggccggcttccctctcctcctcaccctcctcactcactgtgcagggtcttgggcccagtctgtgctgactcagccgccctcagcgtctgggacccccgggcagagggtcaccatctcttgttctggaagcagctccaacatcggaagtaattttgtatactggtaccagcagctcccaggaacggcccccaaactcctcatatataggaataatcagcggccctcaggggtccctgaccgattctctggctccaggtctggcacctcagcctccctggccatcagtggactccggtccgaggatgaggctgattattactgtgcagcatgggatgacagcctgggaggccattatgtcttcggaactgggaccaaggtcaccgtcctt

SEQ ID NO: 11 - protein, 15 aa, Artificial Sequence
  Description of Artificial Sequence Synthetic peptide
GGGGSGGGGSGGGGS

SEQ ID NO: 12 - DNA, 45 bp, Artificial Sequence
  Description of Artificial Sequence Synthetic oligonucleotide
ggcggcggcggatctggaggtggtggctcaggtggcggaggctcc

SEQ ID NO: 13 - protein, 23 aa, Unknown
  Description of Unknown CD8 signal sequence
TAMALPVTALLLPLALLLHAARP

SEQ ID NO: 14 - DNA, 69 bp, Unknown
  Description of Unknown CD8 signal sequence
actgccatggccctgccagtaacggctctgctgctgccacttgctctgctcctccatgcagccaggcct

SEQ ID NO: 15 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 16 - DNA, 321 bp, Homo sapiens
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 17 - protein, 235 aa, Unknown
  Description of Unknown CD8 sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 18 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 19 - DNA, 78 bp, Homo sapiens
ctaattgtgggaatcgttgttggtctcctccttgctgcccttgttgctggtgtcgtctactggctgtacatgaagaag

SEQ ID NO: 20 - DNA, 116 bp, Homo sapiens
accaactggagagaacagtaaactccttgaatgtctctgctataagtattccagaacacgatgaggcagacgagataagtgatgaaaacagagaaaaggtgaatgaccaggcaaaa

SEQ ID NO: 21 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 336 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 24 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 25 - DNA, 336 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 26 - protein, 22 aa, Unknown
  Description of Unknown ITAM1 sequence
QNQLYNELNLGRREEYDVLDKR

SEQ ID NO: 27 - DNA, 66 bp, Unknown
  Description of Unknown ITAM1 sequence
cagaaccagctctataacgagctcaatctaggacgaagagaggagtacgatgttttggacaagaga

SEQ ID NO: 28 - protein, 22 aa, Artificial Sequence
  Description of Artificial Sequence Synthetic peptide
QNQLFNELNLGRREEFDVLDKR

SEQ ID NO: 29 - DNA, 66 bp, Artificial Sequence
  Description of Artificial Sequence Synthetic oligonucleotide
cagaaccagctctttaacgagctcaatctaggacgaagagaggagttcgatgttttggacaagaga

SEQ ID NO: 30 - protein, 23 aa, Unknown
  Description of Unknown ITAM2 sequence
QEGLYNELQKDKMAEAYSEIGMK

SEQ ID NO: 31 - DNA, 69 bp, Unknown
  Description of Unknown ITAM2 sequence
caggaaggcctgtacaatgaactgcagaaagataagatggcggaggcctacagtgagattgggatgaaa

SEQ ID NO: 32 - protein, 23 aa, Artificial Sequence
  Description of Artificial Sequence Synthetic peptide
QEGLFNELQKDKMAEAFSEIGMK

SEQ ID NO: 33 - DNA, 69 bp, Artificial Sequence
  Description of Artificial Sequence Synthetic oligonucleotide
caggaaggcctgttcaatgaactgcagaaagataagatggcggaggccttcagtgagattgggatgaaa

SEQ ID NO: 34 - protein, 22 aa, Unknown
  Description of Unknown ITAM3 sequence
HDGLYQGLSTATKDTYDALHMQ

SEQ ID NO: 35 - DNA, 66 bp, Unknown
  Description of Unknown ITAM3 sequence
cacgatggcctttaccagggtctcagtacagccaccaaggacacctacgacgcccttcacatgcag

SEQ ID NO: 36 - protein, 22 aa, Artificial Sequence
  Description of Artificial Sequence Synthetic peptide
HDGLFQGLSTATKDTFDALHMQ

SEQ ID NO: 37 - DNA, 66 bp, Artificial Sequence
  Description of Artificial Sequence Synthetic oligonucleotide
cacgatggccttttccaggggctcagtacagccaccaaggacaccttcgacgcccttcacatgcag

SEQ ID NO: 38 - protein, 5 aa, Unknown
  Description of Unknown BRS1 sequence
KRRGR

SEQ ID NO: 39 - DNA, 15 bp, Unknown
  Description of Unknown BRS1 sequence
aagagacgtggccgg

SEQ ID NO: 40 - protein, 5 aa, Unknown
  Description of Unknown BRS2 sequence
KPRRK

SEQ ID NO: 41 - DNA, 15 bp, Unknown
  Description of Unknown BRS2 sequence
aagccgagaaggaag

SEQ ID NO: 42 - protein, 8 aa, Unknown
  Description of Unknown BRS3 sequence
KGERRRGK

SEQ ID NO: 43 - DNA, 24 bp, Unknown
  Description of Unknown BRS3 sequence
aaaggcgagcgccggaggggcaag

SEQ ID NO: 44 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 45 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 46 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 47 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 48 - DNA, 123 bp, Mus musculus
aatagtagaaggaacagactccttcaaagtgactacatgaacatgactccccggaggcctgggctcactcgaaagccttaccagccctacgcccctgccagagactttgcagcgtaccgcccc

SEQ ID NO: 49 - protein, 41 aa, Mus musculus
NSRRNRLLQSDYMNMTPRRPGLTRKPYQPYAPARDFAAYRP

SEQ ID NO: 50 - DNA, 123 bp, Mus musculus; identical to SEQ ID NO: 48

SEQ ID NO: 51 - protein, 41 aa, Homo sapiens
RSKRSRLLHSDYMNMTPRRPGPTRKHYQPYAPPRDFAAYRS

SEQ ID NO: 52 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

(19) United States
(12) Patent Application Publication
(10) Pub. No.: US 2021/0246221 A1
(43) Pub. Date: Aug. 12, 2021

(54) CHIMERIC ANTIGEN RECEPTOR TARGETING SIALYL LEWIS A AND USES THEREOF

(71) Applicants: MEMORIAL SLOAN-KETTERING CANCER CENTER, New York, NY (US); BIONTECH RESEARCH AND DEVELOPMENT, INC., Cambridge, MA (US)

(72) Inventors: Michel Sadelain, New York, NY (US); David J. Hansen, Escondido, CA (US)

(73) Assignees: MEMORIAL SLOAN-KETTERING CANCER CENTER, New York, NY (US); BIONTECH RESEARCH AND DEVELOPMENT, INC., Cambridge, MA (US)

(21) Appl. No.: 17/233,994

(22) Filed: Apr. 19, 2021

Related U.S. Application Data
(63) Continuation of application No. PCT/US2019/057017, filed on Oct. 18, 2019.
(60) Provisional application No. 62/748,198, filed on Oct. 19, 2018.

(51) Int. Cl. C07K 16/30; C07K 14/725; C07K 14/705; A61K 35/17; A61P 35/00

(52) U.S. Cl. CPC C07K 16/3076; C07K 14/7051; C07K 14/70521; C07K 2317/622; A61P 35/00; C07K 2319/03; A61K 35/17

(57) ABSTRACT

The presently disclosed subject matter provides for methods and compositions for treating cancer (e.g., pancreatic cancer). It relates to antigen recognizing receptor (e.g., chimeric antigen receptors (CARs)) that specifically target Sialyl Lewis A (e.g., human Sialyl Lewis A), and immunoresponsive cells comprising such CARs. The presently disclosed Sialyl Lewis A-specific CARs have enhanced immune-activating properties, including anti-tumor activity.

### CROSS-REFERENCE TO RELATED APPLICATIONS

This application is a Continuation of International Patent Application No. PCT/US2019/057017, filed on Oct. 18, 2019, which claims priority to U.S. Provisional Application No. 62/748,198, filed on Oct. 19, 2018, the contents of each of which are incorporated by reference in their entirety, and to each of which priority is claimed.

### SEQUENCE LISTING

The specification further incorporates by reference the Sequence Listing submitted herewith via EFS on Apr. 19, 2021. Pursuant to 37 C.F.R. § 1.52(e)(5), the Sequence Listing text file, identified as 0727341246SL.txt, is 36,876 bytes and was created on Apr. 19, 2021. The Sequence Listing electronically filed herewith, does not extend beyond the scope of the specification and thus does not contain new matter.

### INTRODUCTION

The presently disclosed subject matter provides for methods and compositions for treating cancer (e.g., pancreatic cancer). It relates to chimeric antigen receptors (CARs) that specifically target Sialyl Lewis A. The presently disclosed subject matter further provides immunoresponsive cells comprising such CARs, and methods of using such CARs and such cells for treating cancers (e.g., pancreatic cancer).

### BACKGROUND

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating hematologic malignancies (Dunbar et al., Science (2018); 359). Responses to CAR therapy targeting solid tumors have to date been relatively scarce (Sadelain et al., Nature (2017); 545:423-431). One of the challenges to overcome in all cancers and especially solid tumors is antigen heterogeneity. Whereas all or most B cell malignancies express CD19 (Brentjens et al., Nat Med (2003); 9:279-286), numerous potential CAR targets are only expressed in a fraction of all tumor cells within a patient, posing the risk of antigen escape. Low-level antigen expression may also result in resistance to CAR therapy (Fry et al., Nat Med (2018); 24:20-28). Targeting two or more antigens can be implemented in the event of a defined escape population or clone (Wilkie et al., J Clin Immunol (2012); 32:1059-1070; Kloss et al., Nat Biotechnol (2013); 31:71-75; Ruella et al., J Clin Invest (2016); 126:3814-3826; Hegde et al., J Clin Invest (2016); 126:3036-3052; Zah et al., Cancer Immunol Res 2016; 4:498-508), but other approaches are needed to overcome greater or undefined target heterogeneity. Accordingly, there are needs for novel therapeutic strategies to design CARs targeting antigens that are highly expressed in solid tumor cells and for strategies capable of inducing potent anti-cancer effect with minimal toxicity.

### SUMMARY

The presently disclosed subject matter generally provides chimeric antigen receptor (CAR) targeting Sialyl Lewis A.

In certain embodiments, the CAR comprises an extracellular antigen-binding domain, a transmembrane domain and an intracellular domain, wherein the extracellular antigen-binding domain cross-competes for binding to Sialyl Lewis A with a reference antibody or an antigen-binding portion thereof. In some such embodiments, the reference antibody or antigen-binding portion thereof that binds to Sialyl Lewis A comprises a heavy chain variable region comprising one, two, or three heavy chain complementarity determining regions (CDR1, CDR2 and/or CDR3) and a light chain variable region comprising one, two, or three light chain CDRs (CDR1, CDR2 and/or CDR3), wherein the heavy chain CDR1, CDR2 and CDR3 and the light chain CDR1, CDR2 and CDR3 are selected from the heavy chain CDR1, CDR2 and CDR3 and the light chain CDR1, CDR2 and CDR 3 of any one of the antibodies disclosed in U.S. Pat. No. 9,475,874, the content of which is incorporated by reference in its entirety.

In certain embodiments, the CAR comprises an extracellular antigen-binding domain, a transmembrane domain and an intracellular domain, wherein the extracellular antigen-binding domain cross-competes for binding to Sialyl Lewis A with a reference antibody or an antigen-binding portion thereof, wherein the reference antibody or antigen-binding portion thereof comprises: a heavy chain variable region CDR1 comprising the amino acid sequence set forth in SEQ ID NO: 1; a heavy chain variable region CDR2 comprising the amino acid sequence set forth in SEQ ID NO: 2; a heavy chain variable region CDR3 comprising the amino acid sequence set forth in SEQ ID NO: 3; a light chain variable region CDR1 comprising the amino acid sequence set forth in SEQ ID NO: 4; a light chain variable region CDR2 comprising the amino acid sequence set forth in SEQ ID NO: 5 and a light chain variable region CDR3 comprising the amino acid sequence set forth in SEQ ID NO: 6.

In certain embodiments, the CAR comprises an extracellular antigen-binding domain, a transmembrane domain and an intracellular domain, wherein the extracellular antigen-binding domain binds to the same or overlapping epitope on Sialyl Lewis A as a reference antibody or an antigen-binding portion thereof, wherein the reference antibody or antigen-binding portion thereof comprises: a heavy chain variable region CDR1 comprising the amino acid sequence set forth in SEQ ID NO: 1; a heavy chain variable region CDR2 comprising the amino acid sequence set forth in SEQ ID NO: 2; a heavy chain variable region CDR3 comprising the amino acid sequence set forth in SEQ ID NO: 3; a light chain variable region CDR1 comprising the amino acid sequence set forth in SEQ ID NO: 4; a light chain variable region CDR2 comprising the amino acid sequence set forth in SEQ ID NO: 5 and a light chain variable region CDR3 comprising the amino acid sequence set forth in SEQ ID NO: 6.

In certain embodiments, the CAR comprises an extracellular antigen-binding domain, a transmembrane domain and an intracellular domain, wherein the extracellular antigen-binding domain specifically binds to Sialyl Lewis A, and comprises: a heavy chain variable region CDR3 comprising the amino acid sequence set forth in SEQ ID NO: 3 or a conservative modification thereof, and a light chain variable region CDR3 comprising the amino acid sequence set forth in SEQ ID NO: 6 or a conservative modification thereof.

In certain embodiments, the extracellular antigen-binding domain comprises a heavy chain variable region CDR2 comprising the amino acid sequence set forth in SEQ ID NO: 2 or a conservative modification thereof, and a light chain variable region CDR2 comprising the amino acid sequence set forth in SEQ ID NO: 5 or a conservative modification thereof.

In certain embodiments, the extracellular antigen-binding domain comprises a heavy chain variable region CDR1 comprising the amino acid sequence set forth in SEQ ID NO: 1 or a conservative modification thereof, and a light chain variable region CDR1 comprising the amino acid sequence set forth in SEQ ID NO: 4 or a conservative modification thereof.

In certain embodiments, the CAR comprises an extracellular antigen-binding domain, a transmembrane domain and an intracellular domain, wherein the extracellular antigen-binding domain specifically binds to Sialyl Lewis A, and comprises a heavy chain variable region CDR1 comprising the amino acid sequence set forth in SEQ ID NO: 1; a heavy chain variable region CDR2 comprising the amino acid sequence set forth in SEQ ID NO: 2; and a heavy chain variable region CDR3 comprising the amino acid sequence set forth in SEQ ID NO: 3.

In certain embodiments, the CAR comprises an extracellular antigen-binding domain, a transmembrane domain and an intracellular domain, wherein the extracellular antigen-binding domain specifically binds to Sialyl Lewis A, and comprises a light chain variable region CDR1 comprising the amino acid sequence set forth in SEQ ID NO: 4; a light chain variable region CDR2 comprising the amino acid sequence set forth in SEQ ID NO: 5; and a light chain variable region CDR3 comprising the amino acid sequence set forth in SEQ ID NO: 6.

In certain embodiments, the extracellular antigen-binding domain comprises a heavy chain variable region CDR1 comprising the amino acid sequence set forth in SEQ ID NO: 1; a heavy chain variable region CDR2 comprising the amino acid sequence set forth in SEQ ID NO: 2; a heavy chain variable region CDR3 comprising the amino acid sequence set forth in SEQ ID NO: 3; a light chain variable region CDR1 comprising the amino acid sequence set forth in SEQ ID NO: 4; a light chain variable region CDR2 comprising the amino acid sequence set forth in SEQ ID NO: 5 and a light chain variable region CDR3 comprising the amino acid sequence set forth in SEQ ID NO: 6.

In certain embodiments, the CAR comprises an extracellular antigen-binding domain, a transmembrane domain and an intracellular domain, wherein the extracellular antigen-binding domain specifically binds to Sialyl Lewis A, and comprises a heavy chain variable region comprising an amino acid sequence that is at least about 80% homologous (e.g., at least about 80% identical) to SEQ ID NO: 7. In certain embodiments, the extracellular antigen-binding domain comprises a heavy chain variable region comprising the amino acid sequence set forth in SEQ ID NO: 7.

In certain embodiments, the CAR comprises an extracellular antigen-binding domain, a transmembrane domain and an intracellular domain, wherein the extracellular antigen-binding domain specifically binds to Sialyl Lewis A, and comprises a light chain variable region comprising an amino acid sequence that is at least about 80% homologous (e.g., at least about 80% identical) 1 to SEQ ID NO: 8. In certain embodiments, the extracellular antigen-binding domain comprises a light chain variable region comprising the amino acid sequence set forth in SEQ ID NO: 8.

In certain embodiments, the CAR comprises an extracellular antigen-binding domain, a transmembrane domain and an intracellular domain, wherein the extracellular antigen-binding domain specifically binds to Sialyl Lewis A, and comprises:

a) a heavy chain variable region comprising an amino acid sequence that is at least about 80% homologous (e.g., at least about 80% identical) to SEQ ID NO: 7; and

b) a light chain variable region comprising an amino acid sequence that is at least about 80% homologous (e.g., at least about 80% identical) to SEQ ID NO: 8.

In certain embodiments, the extracellular antigen-binding domain comprises: a heavy chain variable region comprising the amino acid sequence set forth in SEQ ID NO: 7; and a light chain variable region comprising the amino acid sequence set forth in SEQ ID NO: 8.

In certain embodiments, the extracellular antigen-binding domain comprises a heavy chain variable region comprising an amino acid sequence that is at least about 80% homologous (e.g., at least about 80% identical) to SEQ ID NO: 7; and a light chain variable region comprising an amino acid sequence that is at least about 80% homologous (e.g., at least about 80% identical) to SEQ ID NO: 8. In certain embodiments, the extracellular antigen-binding domain comprises a heavy chain variable region comprising the amino acid sequence set forth in SEQ ID NO: 7, and a light chain variable region comprising amino acids having a sequence set forth in SEQ ID NO: 8. In certain embodiments, the extracellular antigen-binding domain comprises a heavy chain variable region comprising the amino acid sequence set forth in SEQ ID NO: 7, and a light chain variable region comprising amino acids having a sequence set forth in SEQ ID NO: 8.

In certain embodiments, the extracellular antigen-binding domain comprises a single-chain variable fragment (scFv). In certain embodiments, the extracellular antigen-binding domain comprises a human scFv. In certain embodiments, the extracellular antigen-binding domain comprises a Fab, which is optionally crosslinked. In certain embodiments, the extracellular antigen-binding domain comprises a F(ab)2. In certain embodiments, one or more of the scFV, Fab and F(ab)₂ are comprised in a fusion protein with a heterologous sequence to form the extracellular antigen-binding domain. In certain embodiments, the extracellular antigen-binding domain comprises a linker between a heavy chain variable region and a light chain variable region of the extracellular antigen-binding domain. In certain embodiments, the extracellular antigen-binding domain comprises a signal peptide that is covalently joined to the 5′ terminus of the extracellular antigen-binding domain.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof. In certain embodiments, the intracellular domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide.

In certain embodiments, the intracellular signaling domain of a CAR described herein comprises a wild-type CD3ζ polypeptide or a modified CD3ζ polypeptide. In some embodiments, a modified CD3ζ polypeptide (a) lacks all or part of at least one or more (e.g., 1, 2, or 3) immunoreceptor tyrosine-based activation motifs (ITAMs), wherein an ITAM may be or comprise ITAM1, ITAM2, and/or ITAM3; and/or (b) lacks all or part of at least one or more (e.g., 1, 2, or 3) basic-rich stretch (BRS) regions, wherein a BRS region may be or comprise BRS1, BRS2, and BRS3. In certain embodiments, a modified CD3ζ polypeptide included in an intracellular signaling domain of a CAR described herein comprises at least one or more of the following features:

a) lacks ITAM2 or a portion thereof, optionally further lacks i) ITAM3 or a portion thereof, and/or ii) ITAM1 or a portion thereof;

b) lacks ITAM1 or a portion thereof, optionally further lacks ITAM3 or a portion thereof;

c) lacks ITAM3 or a portion thereof;

d) comprises a deletion of ITAM2 or a portion thereof, optionally further comprises i) a deletion of ITAM3 or a portion thereof, and/or ii) a deletion of ITAM1 or a portion thereof;

e) comprises a deletion of ITAM1 or a portion thereof, optionally further comprises a deletion of ITAM3 or a portion thereof; and/or

f) comprises a deletion of ITAM3 or a portion thereof.

In certain embodiments, a modified CD3ζ polypeptide included in an intracellular signaling domain of a CAR described herein comprises at least one or more of the following features:

a) lacks BRS2 or a portion thereof, and optionally further lacks i) BRS3 or a portion thereof, and/or ii) BRS1 or a portion thereof;

b) lacks BRS1 or a portion thereof, and optionally further lacks BRS3 or a portion thereof;

c) lacks BRS3 or a portion thereof; and/or

d) lacks BRS1 or portion thereof, BRS2 or portion thereof, and BRS3 or a portion thereof;

e) comprises a deletion of BRS2 or a portion thereof, and optionally further comprises i) a deletion of BRS3 or a portion thereof, and/or ii) a deletion of BRS1 or a portion thereof;

f) comprises a deletion of BRS1 or a portion thereof, and optionally further comprises a deletion of BRS3 or a portion thereof;

g) comprises a deletion of BRS3 or a portion thereof; and/or

h) comprises a deletion of BRS1 or portion thereof, BRS2 or portion thereof, and BRS3 or a portion thereof.

In certain embodiments, a modified CD3ζ polypeptide included in an intracellular signaling domain of a CAR described herein lacks ITAM2, ITAM3, BRS2, and BRS3, or comprises a deletion of ITAM2, ITAM3, BRS2, and BRS3.

In certain embodiments, a transmembrane domain of a CAR described herein is or comprises a native or modified transmembrane domain of a molecule selected from the group consisting of a CD8 polypeptide, a CD28 polypeptide, a CD3ζ polypeptide, a CD4 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide and combinations thereof.

In certain embodiments, a CAR described herein further comprises a hinge/spacer region, e.g., between an extracellular antigen-binding domain and a transmembrane domain of the CAR. In some embodiments, such a hinge/spacer region is or comprises a native or modified hinge/spacer region of a molecule selected from the group consisting of a CD8 polypeptide, a CD28 polypeptide, a CD3ζ polypeptide, a CD4 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide and combinations thereof.

In certain embodiments, a CAR described herein comprises a transmembrane domain and a hinge/spacer region, both of which are derived from the same molecule. For example, in certain embodiments, a CAR described herein comprises:

a) a hinge/spacer region of a CD28 polypeptide and a transmembrane domain of a CD28 polypeptide;

b) a hinge/spacer region of a CD84 polypeptide and a transmembrane domain of a CD84 polypeptide;

c) a hinge/spacer region of a CD166 polypeptide and a transmembrane domain of a CD166 polypeptide;

d) a hinge/spacer region of a CD8a polypeptide and a transmembrane domain of a CD8a polypeptide; or

e) a hinge/spacer region of a CD8b polypeptide and a transmembrane domain of a CD8b polypeptide.

In certain embodiments, the CAR comprises a hinge/spacer region of a CD166 polypeptide and a transmembrane domain of a CD166 polypeptide. In certain embodiments, a CAR described herein comprises a transmembrane domain and a hinge/spacer region, which are each derived from a different molecule. For example, in certain embodiments, such a CAR may comprise a hinge/spacer region of a CD28 polypeptide and a transmembrane domain of an ICOS polypeptide.

In certain embodiments, CARs described herein are recombinantly expressed, or expressed from a vector. In certain embodiments, such a vector is a retroviral vector (e.g., a γ-retroviral vector).

The presently disclosed subject matter further provides immunoresponsive cells comprising a CAR disclosed herein. In certain embodiments, such an immunoresponsive cell is transduced with a vector comprising a CAR described herein. In certain embodiments, a CAR described herein is constitutively expressed on the surface of an immunoresponsive cell. Examples of immunoresponsive cells that are useful in accordance with the present disclosure include, but are not limited to, a T cell, a Natural Killer (NK) cell, a human embryonic stem cell, a lymphoid progenitor cell, a T cell-precursor cell, and a pluripotent stem cell (e.g., from which lymphoid cells may be differentiated). In certain embodiments, an immunoresponsive cell that comprises a CAR described herein is a T cell. In certain embodiments, such a T cell is selected from the group consisting of a cytotoxic T lymphocyte (CTL), a regulatory T cell, and a central memory T cell.

The presently disclosed subject matter also provides nucleic acid molecules encoding a CAR disclosed herein.

The presently disclosed subject matter further provides vectors each comprising a nucleic acid molecule disclosed herein. In certain embodiments, such a vector is a retroviral vector (e.g., a γ-retroviral vector).

The presently disclosed subject matter also provides a host cell expressing a nucleic acid molecule comprising a nucleic acid sequence that encodes a CAR as disclosed herein. In certain embodiments, such a host cell is a T cell.

Furthermore, the presently disclosed subject matter provides methods for producing an immunoresponsive cell that binds to Sialyl Lewis A. In certain embodiments, such a method comprises introducing into an immunoresponsive cell a nucleic acid sequence that encodes a CAR disclosed herein.

In addition, the presently disclosed subject matter provides a composition comprising an immunoresponsive cell that binds to Sialyl Lewis A (e.g., ones disclosed herein). In certain embodiments, such a composition is a pharmaceutical composition comprising an immunoresponsive cell that binds to Sialyl Lewis A (e.g., ones disclosed herein) and a pharmaceutically acceptable carrier.

Furthermore, the presently disclosed subject matter provides methods of treating and/or preventing a malignant growth in a subject. In certain embodiments, the method comprises administering to the subject an effective amount of immunoresponsive cells disclosed herein, or a composition disclosed herein. In certain embodiments, the malignant growth is pancreatic cancer. In certain embodiments, the method reduces or eradicates the tumor burden in the subject. In certain embodiments, the subject is a human.

The presently disclosed subject matter also provides kits for treating and/or preventing a malignant growth. In certain embodiments, the kit comprises the immunoresponsive cell disclosed herein. In certain embodiments, the kit further comprises written instructions for using the immunoresponsive cell for treating a subject having a neoplasia. In certain embodiments, the malignant growth is pancreatic cancer.

### BRIEF DESCRIPTION OF THE FIGURES

The following Detailed Description, given by way of example but not intended to limit the invention to specific embodiments described, may be understood in conjunction with the accompanying drawings.

FIGS. 1A-1F depict that radiation therapy (RT) sensitizes pancreatic cancer to CAR T cell killing without affecting target antigen expression. FIG. 1A shows tumor cell viability 48 hours after exposure to various doses of radiation. FIG. 1B shows Capan2 pancreatic cancer cells exposed to low dose RT (2 Gy), and 48 hours later incubated with CAR T cells at indicated ratios for 18 hours, after which percent killing was determined. FIG. 1C shows target antigen expression levels unchanged 48 hours after RT. FIG. 1D shows transcriptome analysis of target cells six hours after RT reveals a number of apoptotic pathways significantly affected. FIG. 1E shows TRAIL mRNA expression and protein levels in the media of CAR T cells after exposure to target antigen (Sialyl Lewis A (Le^{A})-expressing capan2 cells). FIG. 1F shows TRAIL protein quantified in the media of LBBz and L(del) CAR T cells grown on target cells expressing or not expressing the target antigen. LFC=log 2 fold change; E:T=effector:target.

FIGS. 2A-2C depict that TRAIL expressed by activated CAR T cells is functionally significant against antigen-negative tumor cells in a heterogeneous tumor population exposed to low-dose radiation. FIG. 2A shows CAR-activated T cells produce TRAIL, which acts upon radiation-sensitized antigen-positive and antigen-negative tumor cells. FIGS. 2B-2C show Ag⁺ cells were mixed with luciferase-expressing Ag⁻ cells at a ratio of 75:25, exposed to low-dose RT, and cocultured with the indicated CAR T cells for four days, followed by quantification of Ag⁺ cell killing.

FIGS. 3A-3B depict that sensitizing RT transcriptionally primes pancreatic cancer cells for TRAIL-induced death. FIG. 3A shows RNA expression levels of signaling molecules known to mediate various TRAIL responses, including survival and migration, tumor-supportive inflammation, necroptosis, apoptosis, and death receptor endocytosis, were quantified by RNAseq before and after RT exposure to Capan2 pancreatic cancer cells in three biologic replicates. Significantly induced and downregulated molecules are shown in red and green, respectively, with magnitude represented by color gradient. Molecules in gray were not significantly changed. FIG. 3B shows CTV-labeled Ag⁻ cells were exposed to RT two days before coculture with unlabeled Ag⁺ cells, annexin-V 595, and TRAIL^{-/-} or TRAIL^{wt} CAR T cells. Cultures were monitored by live video microscopy and Ag⁻ cell apoptosis quantified over time.

FIGS. 4A-4M depict that sensitizing RT allows CAR T cells to eliminate heterogeneous PDAC in vivo. FIG. 4A shows Capan2 tumor cells mixed at 75:25 LeA(+):(−), then injected into the pancreas of NSG mice. After tumor established for 9 days, mice were given RT, followed by CAR T cells. FIG. 4B shows waterfall plot of tumor volume change at time of death among different treatment groups. FIGS. 4C-4H illustrate that BLI was performed weekly. FIGS. 4I-4K show that T cell infiltration of tumor from CAR or RT+CAR treated mice was determined using BLI T cell imaging (detecting G-Luc on the transduced T cell) over the first 19 days (FIG. 4I), and by IHC from mice sacrificed on day 21 (FIGS. 4J-4K, all ns). FIG. 4L shows tumors in mice that progressed display reduced target antigen expression over time by FACS. FIG. 4M depicts BLI of mice treated with RT+L(del) or RT+L(del)-TRAIL CAR T cells.

FIGS. 5A-5G depict that Outcome of DLBCL patient with heterogeneous tumor treated with palliative RT and CAR T cells. FIG. 5A shows that total body or local RT was delivered to mice harboring heterogeneous tumor of the pancreas using image-guided radiation, followed by CAR T cells. FIG. 5B-5D shows that tumor burden was monitored by BLI. FIGS. 5E-5F illustrate patient biopsy before CAR T cell treatment examined for CD19 by IHC (FIG. 5E) and flow cytometry (FIG. 5F). FIG. 5G shows FDG-PET scan before, and 1, 2, and 6 months post palliative leg RT and systemic 1928z CAR T cells.

FIGS. 6A-6C depict CAR targeting LeA specifically lyses cells that express LeA. FIG. 6A shows LBBz CAR T cell design, containing membrane-bound G-Luc for imaging. FIG. 6B shows that endogenous LeA expression on PC3, Capan2, and BxPC3 cells was examined by flow cytometry. FIG. 6C illustrates PC3, Capan2, or BxPC3 cells mixed with LBBz or L28z CAR or untransduced T cells at various effector:target ratios for 18 hours followed by quantification of target cell killing.

FIG. 7 depicts that Capan2 cells were FACS sorted into LeA⁺ and LeA⁻ populations, then mixed at a ratio of 75:25 LeA+/− tumor cells. LeA⁻ sorted Capan2 cells remain LeA⁻ over time.

FIG. 8 depicts that TRAIL^{wt} or CRISPR knockout CAR T cells were stimulated on their target, then TRAIL mRNA was quantified and displayed relative to wt unstimulated CAR T cells.

FIG. 9 depicts fold mRNA change of molecules known to mediate various TRAIL processes, including survival and migration, tumor-supportive inflammation, necroptosis, apoptosis, and death receptor endocytosis, following low dose RT. Molecules with an adjusted p-value <0.05 are shown.

FIG. 10 depicts Typical T cell profile after CAR transduction and TCR knockout, before in vivo injection.

FIG. 11 depicts that CAR T cell tumor infiltration, quantified by CTZ T-cell bioluminescent imaging over time, shows both TRAIL-knockout LBBz and L(del) CAR T cells accumulate in the pancreatic tumors over time.

FIGS. 12A-12B depict that CAR T cells persist in vivo, penetrate tumor, and deplete Ag⁺ tumor cells in mice harboring heterogeneous Ag+/− pancreatic cancer. FIG. 12A depicts that cells isolated from blood, spleen, and tumor from mice treated with CAR T cells 6 weeks prior were analyzed for CAR T cells content (pure T cell population controls show beneath). FIG. 12B shows IHC of LeA expression from pancreatic tumor at different time points post CAR T cell treatment, showing depletion of target antigen-expressing tumor cells throughout therapy.

FIG. 13 depicts T cell accumulation in the tumor of mice treated with total body or local RT. LBBz CAR T cells were quantified in the pancreatic tumor using bioluminescence imaging over time in mice treated with local, total body (TBI), or no RT followed by CAR T cells.

### DETAILED DESCRIPTION OF CERTAIN EXEMPLARY EMBODIMENTS

The presently disclosed subject matter provides antigen-binding proteins such as chimeric antigen receptors (CARs) targeting Sialyl Lewis A.

The presently disclosed subject matter also provides immunoresponsive cells (e.g., a T cell (e.g., a cytotoxic T lymphocyte (CTL), a regulatory T cell, a central memory T cell, etc.), a Natural Killer (NK) cell, a human embryonic stem cell, a lymphoid progenitor cell, a T cell-precursor cell, and a pluripotent stem cell from which lymphoid cells may be differentiated) comprising the Lewis A-targeted CARs, and/or nucleic acid(s) that encode them, and methods of using such immunoresponsive cells for treating and/or preventing a tumor, e.g., pancreatic cancer.

### I. Certain Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein interchangeably, the terms “antigen-binding portion”, “antigen-binding fragment”, or “antigen-binding region” of an antibody, refer to the region or portion of an antibody that binds to the antigen and which confers antigen specificity to the antibody; fragments of antigen-binding proteins, for example, antibodies includes one or more fragments of an antibody that retain the ability to specifically bind to an antigen (e.g., an peptide/HLA complex). It has been shown that the antigen-binding function of an antibody can be performed by fragments of a full-length antibody. Examples of antigen-binding portions encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., 1988 Science 242:423-426; and Huston et al., 1988 Proc. Natl. Acad. Sci. 85:5879-5883. These antibody fragments are obtained using conventional techniques known to those of ordinary skill in the art, and the fragments are screened for utility in the same manner as are intact antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. In certain embodiments, the linker comprises amino acids having the sequence set forth in SEQ ID NO:11 as provided below.

GGGGSGGGGSGGGGS [SEQ ID NO:11]

In certain embodiments, the nucleic acid sequence encoding the amino acid sequence of SEQ ID NO: 11 is set forth in SEQ ID NO: 12, which is provided below:

GGCGGCGGCGGATCTGGAGGTGGTGGCTCAGGTGGCGGAGGCTCC [SEQ ID NO: 12]

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et al., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen-binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or even about 99% homologous (e.g., identical) at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

In certain embodiments, the term “cross-compete” or “compete” refers to the situation where binding of an extracellular antigen-binding domain of a presently disclosed CAR to a given antigen, decreases or reduces binding of a reference antibody or an antigen-binding portion thereof, e.g., that comprises the V_{H} and V_{L} CDR1, CDR2, and CDR3 sequences or V_{H} and V_{L} sequences of any one of the presently disclosed scFvs, to the same antigen. The term “cross-compete” or “compete” also refers to the situation where binding of a reference antibody or an antigen-binding portion thereof to a given antigen, decreases or reduces binding of an extracellular antigen-binding domain of a presently disclosed CAR to the same antigen. In certain embodiments, the “cross-competing” or “competing” extracellular antigen-binding domain binds to the same or substantially the same epitope, an overlapping epitope, or an adjacent epitope as the reference antibody or antigen-binding portion thereof.

As used herein, the term “analog” refers to a structurally related polypeptide or nucleic acid molecule having the function of a reference polypeptide or nucleic acid molecule.

As used herein, the term “ligand” refers to a molecule that binds to a receptor. In particular, the ligand binds a receptor on another cell, allowing for cell-to-cell recognition and/or interaction.

As used herein, the term “disease” refers to any condition or disorder that damages or interferes with the normal function of a cell, tissue, or organ. Examples of diseases include neoplasia or pathogen infection of cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

As used herein, the term “specifically binds” or “specifically binds to” or “specifically target” is meant a polypeptide or fragment thereof that recognizes and binds a biological molecule of interest (e.g., a polypeptide), but which does not substantially recognize and bind other molecules in a sample, for example, a biological sample, which includes or expresses a human Sialyl Lewis A. For example, in some embodiments, an extracellular antigen-binding domain of a CAR described herein that interacts with one particular target (e.g., Sialyl Lewis A) when other potential targets are present is said to “bind specifically” to the target (e.g., Sialyl Lewis A) with which it interacts. In some embodiments, specific binding is assessed by detecting or determining degree of association between a target binding moiety and its partner; in some embodiments, specific binding is assessed by detecting or determining degree of dissociation of a target binding moiety-partner complex; in some embodiments, specific binding is assessed by detecting or determining ability of a target binding moiety to compete an alternative interaction between its partner and another entity. In some embodiments, specific binding is assessed by performing such detections or determinations across a range of concentrations.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

### II. Sialyl Lewis A

Sialyl Lewis A (also known as Le^{A}, sialyl Le^{A} and SLe^{A}, CAS No. 92448-22-1) is a tetrasaccharide comprising the sugar sequence of NeuAc(a2-3)Gal(b1-3)[Fuc(a1-4)]GlcNAc. In certain embodiments, Le^{A} comprises a formula of

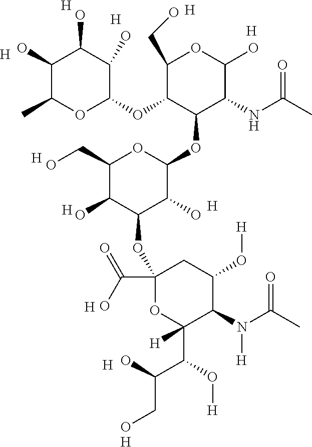

Le^{A} is present on the surface of certain cells and is involved in cell-to-cell recognition processes. It is a surface antigen expressed on tumors, e.g., 75-90% of pancreatic tumors, whereas its expression on normal human tissues is relatively low.

### III. Chimeric Antigen Receptor (CAR)

The present disclosure provides chimeric antigen receptors (CARs) that target a cancer antigen. In many embodiments, the present disclosure provides CARs that target a pancreatic cancer antigen, e.g., Sialyl Lewis A.

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen binding domain (e.g., a single-chain variable fragments (scFv)) fused to a transmembrane domain, fused to cytoplasmic/intracellular domain of the T cell receptor chain. “First generation” CARs typically have the intracellular domain from the CD3ζ-chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3ζ). A person skilled in the art, reading the present disclosure, will recognize that CAR constructs provided herein may be first generation, second generation, or third generation construct(s).

In certain non-limiting embodiments, the extracellular antigen-binding domain of a presently disclosed CAR has a high binding specificity as well as high binding affinity to human Sialyl Lewis A. For example, in such embodiments, the extracellular antigen-binding domain of the CAR (embodied, for example, in a human scFv or an analog thereof) binds to human Sialyl Lewis A with a dissociation constant (K_{d}) of about 2×10⁻⁷ M or less. In certain embodiments, the K_{d} is about 2×10⁻⁷ M or less, about 1×10⁻⁷ M or less, about 5×10⁻⁸M or less, about 2×10⁻⁸M or less, about 1×10⁻⁸M or less, about 9×10⁻⁹ or less, about 8×10⁻⁹ or less, about 7×10⁻⁹ or less, about 6×10⁻⁹ or less, about 5×10⁻⁹ or less, about 4×10⁻⁹ or less, about 3×10⁻⁹ or less, about 2×10⁻⁹ or less, or about 1×10⁻⁹M or less. In certain non-limiting embodiments, the K_{d} is from about 2×10⁻⁸M or less. In certain non-limiting embodiments, the K_{d} is from about 1×10⁻⁸M to about 2×10⁻⁸M. In certain non-limiting embodiments, the K_{d} is from about 1.3×10⁻⁸M or less. In certain non-limiting embodiments, the K_{d} is from about 1.8×10⁻⁸M or less. In certain non-limiting embodiments, the K_{d} is from about 1×10⁻⁹M to about 1×10⁻⁸M.

Binding of the extracellular antigen-binding domain (embodiment, for example, in a human scFv or an analog thereof) of a presently disclosed Sialyl Lewis A-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the Sialyl Lewis A-targeted CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In certain embodiments, the human scFv of a presently disclosed Sialyl Lewis A-targeted CAR is labeled with GFP.

In accordance with the presently disclosed subject matter, the CARs comprise an extracellular antigen-binding domain, a transmembrane domain and an intracellular domain, where the extracellular antigen-binding domain specifically binds to Sialyl Lewis A (e.g., human Sialyl Lewis A). In certain embodiments, the extracellular antigen-binding domain is an scFv. In certain embodiments, the extracellular antigen-binding domain is a Fab, which is optionally crosslinked. In certain embodiments, the extracellular binding domain is a F(ab)₂. In certain embodiments, any of the foregoing molecules may be comprised in a fusion protein with a heterologous sequence to form the extracellular antigen-binding domain. In certain embodiments, the extracellular antigen-binding domain comprises a human scFv that binds specifically to human Sialyl Lewis A. In certain embodiments, the scFv is identified by screening scFv phage library.

Extracellular Antigen Binding Domain of A CAR

In certain embodiments, the extracellular antigen-binding domain of a CAR described herein comprises a heavy variable region comprising one, two, or three CDRs (e.g., CDR1, CDR2 and/or CDR3) of anti-Sialyl-Lewis A antibodies or antibody binding fragments thereof as disclosed in U.S. Pat. No. 9,475,874 (the “'874 patent”), the content of which is incorporated by reference in its entirety for the purpose described herein. Additionally or alternatively, in certain embodiments, the extracellular antigen-binding domain of a CAR described herein comprises a light variable region comprising one, two, or three CDRs (e.g., CDR1, CDR2 and/or CDR3) of anti-Sialyl-Lewis A antibodies or antibody binding fragments thereof as disclosed in the '874 patent, the content of which is incorporated by reference in its entirety for the purpose described herein. For example, Table 2 of the '874 patent sets forth amino acid and nucleic acid sequences of CDRs in heavy chain and light chain of such anti-Sialyl-Lewis A antibodies or antibody binding fragments thereof. A person of skill in the art reading the present disclosure will understand that any of such sequences can be used in accordance with the present disclosure.

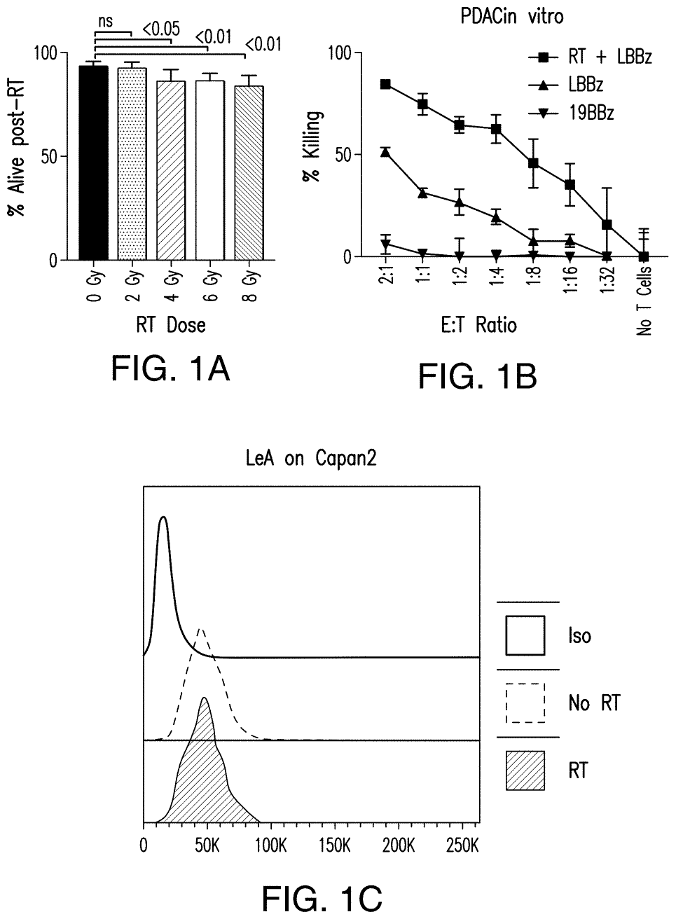

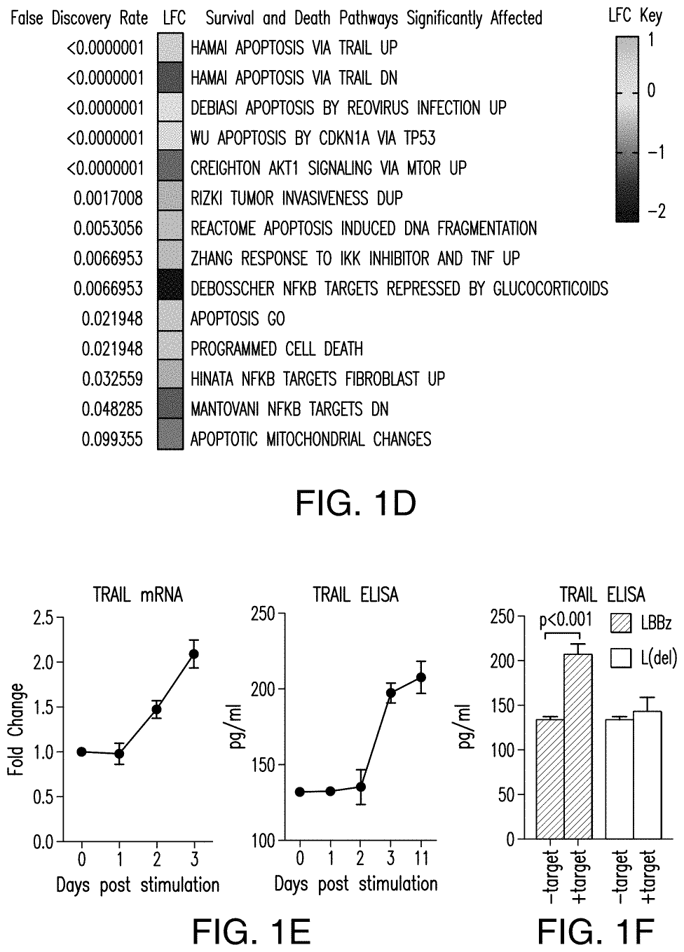

In certain embodiments, the extracellular antigen-binding domain of a CAR described herein comprises (i) a heavy chain variable region of anti-Sialyl-Lewis A antibodies or antibody binding fragments thereof as disclosed in the '874 patent, and/or (ii) a light chain variable region of anti-Sialyl-Lewis A antibodies or antibody binding fragments thereof as disclosed in the '874 patent, the content of which is incorporated by reference in its entirety for the purpose described herein. For example, FIGS. 1-10 of the '874 patent set forth amino acid sequences of V_{H} and V_{L} of such anti-Sialyl-Lewis A antibodies or antibody binding fragments thereof. A person of skill in the art reading the present disclosure will understand that any of such sequences can be used in accordance with the present disclosure. A person of skill in the art will also understood that appropriate substitutions (e.g., conservative substitutions), deletions, insertions, and/or modifications can also be made to such sequences provided that the resulting sequence is at least 70% or more (e.g., at least 80%, at least 90%, at least 95%, at least 98%, at least 99%, or more) identical to a corresponding parent sequence and retains the ability to specifically bind Sialyl-Lewis A.

In certain embodiments, the extracellular antigen-binding domain (e.g., human scFv) comprises a heavy chain variable region comprising the amino acid sequence set forth in SEQ ID NO: 7. An exemplary nucleic acid sequence encoding the amino acid sequence of SEQ ID NO: 7 is set forth in SEQ ID NO: 9. In certain embodiments, the extracellular antigen-binding domain (e.g., human scFv) comprises a light chain variable region comprising the amino acid sequence set forth in SEQ ID NO: 8. An exemplary nucleic acid sequence encoding the amino acid sequence of SEQ ID NO: 8 is set forth in SEQ ID NO: 10. The sequences of SEQ ID NOS:1-10 are described in the following Table 1.

In certain embodiments, the extracellular antigen-binding domain is a human scFv and specifically binds to a Sialyl Lewis A (e.g., a human Sialyl Lewis A), which is designated as scFv 5B1.

In certain embodiments, the extracellular antigen-binding domain is a human scFv. In certain embodiments, the extracellular antigen-binding domain comprises a heavy chain variable region comprising the amino acid sequence set forth in SEQ ID NO:7 and a light chain variable region comprising the amino acid sequence set forth in SEQ ID NO:8, optionally with (iii) a linker sequence, for example a linker peptide, between the heavy chain variable region and the light chain variable region. In certain embodiments, the linker comprises the amino acid sequence set forth in SEQ ID NO:11. In certain embodiments, the extracellular antigen-binding domain is a human scFv-Fc fusion protein or full length human IgG with V_{H} and V_{L} regions or CDRs selected from Table 1.

In certain embodiments, the extracellular antigen-binding domain comprises a V_{H} comprising an amino acid sequence that is at least about 80% (e.g., at least about 85%, at least about 90%, or at least about 95%) homologous (e.g., identical) to the amino acid sequence set forth in SEQ ID NO: 7, as shown in Table 1. For example, the extracellular antigen-binding domain comprises a V_{H} comprising an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% homologous (e.g., identical) to the amino acid sequence set forth in SEQ ID NO: 7. In certain embodiments, the extracellular antigen-binding domain comprises a V_{H} comprising the amino acid sequence set forth in SEQ ID NO:7. In certain embodiments, the extracellular antigen-binding domain comprises a V_{L} comprising an amino acid sequence that is at least about 80% (e.g., at least about 85%, at least about 90%, or at least about 95%) homologous (e.g., identical) to the amino acid sequence set forth in SEQ ID NO: 8, as shown in Table 1. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous (e.g., identical) to the amino acid sequence set forth in SEQ ID NO: 8. In certain embodiments, the extracellular antigen-binding domain comprises a V_{L} comprising the amino acid sequence set forth in SEQ ID NO:8. In certain embodiments, the extracellular antigen-binding domain comprises a V_{H} comprising an amino acid sequence that is at least about 80% (e.g., at least about 85%, at least about 90%, or at least about 95%) homologous (e.g., identical) to the amino acid sequence set forth in SEQ ID NO: 7, and a V_{L} comprising an amino acid sequence that is at least about 80% (e.g., at least about 85%, at least about 90%, or at least about 95%) homologous (e.g., identical) to the amino acid sequence set forth in SEQ ID NO: 8. In certain embodiments, the extracellular antigen-binding domain comprises a V_{H} comprising the amino acid sequence set forth in SEQ ID NO:7 and a V_{L} comprising the amino acid sequence set forth in SEQ ID NO:8.

In certain embodiments, the extracellular antigen-binding domain of a CAR described herein comprises at least one or more (e.g., 1, 2, or 3) heavy chain variable region (V_{H}) CDRs specifically targeting Sialyl-Lewis A. For example, in some embodiments, the extracellular antigen-binding domain of a CAR described herein comprises at least one or more (e.g., 1, 2, or 3) of the following: (i) V_{H} CDR1 comprising the amino acid sequence set forth in SEQ ID NO:1 or a conservative modification thereof, (ii) a V_{H} CDR2 comprising the amino acid sequence set forth in SEQ ID NO:2 or a conservative modification thereof, and (iii) a V_{H} CDR3 comprising the amino acid sequence set forth in SEQ ID NO:3 or a conservative modification thereof, as shown in Table 1. In certain embodiments, the extracellular antigen-binding domain comprises a V_{H} CDR1 comprising the amino acid sequence set forth in SEQ ID NO:1 or a conservative modification thereof, a V_{H} CDR2 comprising the amino acid sequence set forth in SEQ ID NO:2 or a conservative modification thereof, and a V_{H} CDR3 comprising the amino acid sequence set forth in SEQ ID NO:3 or a conservative modification thereof, as shown in Table 1. In certain embodiments, the extracellular antigen-binding domain comprises a V_{H} CDR1 comprising the amino acid sequence set forth in SEQ ID NO:1, a V_{H} CDR2 comprising the amino acid sequence set forth in SEQ ID NO:2, and a V_{H} CDR3 comprising the amino acid sequence set forth in SEQ ID NO:3.

In certain embodiments, the extracellular antigen-binding domain of a CAR described herein comprises at least one or more (e.g., 1, 2, or 3) light chain variable region (V_{L}) CDRs specifically targeting Sialyl-Lewis A. For example, in some embodiments, the extracellular antigen-binding domain of a CAR described herein comprises at least one or more (e.g., 1, 2, or 3) of the following: (i) V_{L} CDR1 comprising the amino acid sequence set forth in SEQ ID NO:4 or a conservative modification thereof, (ii) a V_{L} CDR2 comprising the amino acid sequence set forth in SEQ ID NO: 5 or a conservative modification thereof, and (iii) a V_{L} CDR3 comprising the amino acid sequence set forth in SEQ ID NO: 6 or a conservative modification thereof, as shown in Table 1. In certain embodiments, the extracellular antigen-binding domain comprises a V_{L} CDR1 comprising the amino acid sequence set forth in SEQ ID NO:4 or a conservative modification thereof, a V_{L} CDR2 comprising the amino acid sequence set forth in SEQ ID NO: 5 or a conservative modification thereof, and a V_{L} CDR3 comprising the amino acid sequence set forth in SEQ ID NO: 6 or a conservative modification thereof, as shown in Table 1. In certain embodiments, the extracellular antigen-binding domain comprises a V_{L} CDR1 comprising the amino acid sequence set forth in SEQ ID NO:4, a V_{L} CDR2 comprising the amino acid sequence set forth in SEQ ID NO: 5, and a V_{L} CDR3 comprising the amino acid sequence set forth in SEQ ID NO: 6.

In certain embodiments, the extracellular antigen-binding domain of a CAR described herein comprises a V_{H} CDR1 comprising the amino acid sequence set forth in SEQ ID NO: 1 or a conservative modification thereof and a V_{L} CDR1 comprising the amino acid sequence set forth in SEQ ID NO: 4 or a conservative modification thereof. In certain embodiments, the extracellular antigen-binding domain of a CAR described herein comprises a V_{H} CDR2 comprising the amino acid sequence set forth in SEQ ID NO: 2 or a conservative modification thereof, and a V_{L} CDR2 comprising the amino acid sequence set forth in SEQ ID NO: 5 or a conservative modification thereof. In certain embodiments, the extracellular antigen-binding domain of a CAR described herein comprises a V_{H} CDR3 comprising the amino acid sequence set forth in SEQ ID NO: 3 or a conservative modification thereof, and a V_{L} CDR3 comprising the amino acid sequence set forth in SEQ ID NO: 6 or a conservative modification thereof.

In certain embodiments, the extracellular antigen-binding domain of a CAR described herein comprises a V_{H} CDR1 comprising the amino acid sequence set forth in SEQ ID NO: 1 or a conservative modification thereof, a V_{H} CDR2 comprising the amino acid sequence set forth in SEQ ID NO: 2 or a conservative modification thereof, a V_{H} CDR3 comprising the amino acid sequence set forth in SEQ ID NO: 3 or a conservative modification thereof, a V_{L} CDR1 comprising the amino acid sequence set forth in SEQ ID NO: 4 or a conservative modification thereof, a V_{L} CDR2 comprising the amino acid sequence set forth in SEQ ID NO: 5 or a conservative modification thereof, and a V_{L} CDR3 comprising the amino acid sequence set forth in SEQ ID NO: 6 or a conservative modification thereof.

In certain embodiments, the extracellular antigen-binding domain comprises a V_{H} CDR1 comprising the amino acid sequence set forth in SEQ ID NO: 1, a V_{H} CDR2 comprising the amino acid sequence set forth in SEQ ID NO: 2, a V_{H} CDR3 comprising the amino acid sequence set forth in SEQ ID NO: 3, a V_{L} CDR1 comprising the amino acid sequence set forth in SEQ ID NO: 4, a V_{L} CDR2 comprising the amino acid sequence set forth in SEQ ID NO: 5, and a V_{L} CDR3 comprising the amino acid sequence set forth in SEQ ID NO: 6.

In certain embodiments, CDRs are presented below (e.g., according to Kabat numbering).

TABLE 1

| | Sialyl Lewis A | | |
|---|---|---|---|
| | CDRs | | |
| Antigen | 1 | 2 | 3 |
| V_{H} | GFTFEAYA | INWNSGRI | AKDIRRFSTGGAEFEY |
| | [SEQ ID NO: 1] | [SEQ ID NO: 2] | [SEQ ID NO: 3] |
| V_{L} | SSNIGSNF | RNN | AAWDDSLGGHYV |
| | [SEQ ID NO: 4] | [SEQ ID NO: 5] | [SEQ ID NO: 6] |

| | |
|---|---|
| V_{H} | MEFGLSWLFL VAILKGVQCQ VQLVESGGGS VQPGRSLRLS CEASGFTFEA YAMHWVRQPP |
| Poly- | GKGLEWVSSI NWNSGRIAYA DSVKGRFTIS RDNARNSLYL QMNSLRLEDT AFYYCAKDIR |
| peptide | RFSTGGAEFE YWGQGTLVTV SS [SEQ ID NO: 7] |
| Nucleic | ATGGAGTTTG GGCTGAGCTG GCTTTTTCTT GTGGCTATTT TAAAAGGCGT ACAGTGCCAG |
| acid | GTGCAGCTGG TGGAGTCTGG GGGAGGCTCG GTGCAGCCTG GCAGGTCCCT GAGACTCTCC |
| encoding | TGTGAAGCCT CTGGATTCAC CTTTGAGGCC TATGCCATGC ACTGGGTCCG GCAACCTCCA |
| a V_{H} | GGGAAGGGCC TGGAGTGGGT CTCAAGTATT AATTGGAATA GTGGTCGCAT AGCCTATGCG |
| Poly- | GACTCTGTGA AGGGCCGATT CACCATCTCC AGAGACAACG CCAGGAATTC CCTGTATCTG |
| peptide | CAAATGAACA GTCTGAGACT TGAGGACACG GCCTTCTATT ACTGTGCAAA AGATATACGG |
| | AGGTTTAGTA CCGGGGGGGC GGAGTTTGAG TACTGGGGCC AGGGAACCCT GGTCACCGTC |
| | TCCTCA [SEQ ID NO: 9] |
| V_{L} | MAGFPLLLTL LTHCAGSWAQ SVLTQPPSAS GTPGQRVTIS CSGSSSNIGS NFVYWYQQLP |
| Poly- | GTAPKLLIYR NNQRPSGVPD RFSGSRSGTS ASLAISGLRS EDEADYYCAA WDDSLGGHYV |
| peptide | FGTGTKVTVL [SEQ ID NO: 8] |
| Nucleic | ATGGCCGGCT TCCCTCTCCT CCTCACCCTC CTCACTCACT GTGCAGGGTC TTGGGCCCAG |
| acid | TCTGTGCTGA CTCAGCCGCC CTCAGCGTCT GGGACCCCCG GGCAGAGGGT CACCATCTCT |
| encoding | TGTTCTGGAA GCAGCTCCAA CATCGGAAGT AATTTTGTAT ACTGGTACCA GCAGCTCCCA |
| a V_{L} | GGAACGGCCC CCAAACTCCT CATATATAGG AATAATCAGC GGCCCTCAGG GGTCCCTGAC |
| Poly- | CGATTCTCTG GCTCCAGGTC TGGCACCTCA GCCTCCCTGG CCATCAGTGG ACTCCGGTCC |
| peptide | GAGGATGAGG CTGATTATTA CTGTGCAGCA TGGGATGACA GCCTGGGAGG CCATTATGTC |
| | TTCGGAACTG GGACCAAGGT CACCGTCCTT [SEQ ID NO: 10] |

As used herein, the term “a conservative modification,” or “a conservative sequence modification” refers to an amino acid modification that does not significantly affect or alter the binding characteristics (e.g., specificity and/or affinity) of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

For example, in some embodiments, a conservative modification of V_{H} and/or V_{L} amino acid sequences (e.g., SEQ ID NOs: 1-10 as set forth in Table 1) included in a CAR described herein are amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the specified sequences that contain at least one or more (e.g., at least 1, at least 2, at least 3, at least 4, at least 5, at least 6, at least 7, at least 8, at least 9, at least 10, or more) substitutions (e.g., conservative substitutions), insertions, and/or deletions relative to the specified sequence(s), but retain the ability to bind to Sialyl Lewis A (e.g., human Sialyl Lewis A). In some embodiments, such a conservation modification of a V_{H} and/or V_{L} amino acid sequence (e.g., SEQ ID NOs: 1-10 as set forth in Table 1) included in a CAR described herein retain at least 70% or more, including, e.g., at least 80%, at least 90%, at least 95%, or more, and up to 100%, of binding affinity for Sialyl-Lewis A of a corresponding non-modified V_{H} and/or V_{L} amino acid sequence. For example, in certain embodiments, the extracellular antigen-binding domain specifically binds to Sialyl Lewis A (e.g., human Sialyl Lewis A) with a binding affinity (K_{d}) of about 3×10⁻⁸ or less. In certain embodiments, the extracellular antigen-binding domain specifically binds to Sialyl Lewis A (e.g., human Sialyl Lewis A) with a binding affinity (K_{d}) of about 2×10⁻⁸ or less. In certain embodiments, the extracellular antigen-binding domain specifically binds to Sialyl Lewis A (e.g., human Sialyl Lewis A) with a binding affinity (K_{d}) of about 1.3×10⁻⁸ or less. In certain embodiments, the extracellular antigen-binding domain specifically binds to Sialyl Lewis A (e.g., human Sialyl Lewis A) with a binding affinity (K_{d}) of about 1.8×10⁻⁸ or less. In certain embodiments, the extracellular antigen-binding domain binds to Sialyl Lewis A (e.g., human Sialyl Lewis A) with a binding affinity (K_{d}) of from about 1×10⁻⁹ to about 1×10⁻⁷. In certain embodiments, the extracellular antigen-binding domain binds to Sialyl Lewis A (e.g., human Sialyl Lewis A) with a binding affinity (K_{d}) of from about 1×10⁻⁸ to about 2×10⁻⁸. In certain embodiments, a total of 1 to 10 amino acids are substituted, inserted and/or deleted in SEQ ID NOs: 7 or 8. In certain embodiments, substitutions, insertions, or deletions occur in regions outside the CDRs (e.g., in the FRs) of the extracellular antigen-binding domain. A person skilled in the art, reading Table 1 presented herein, will be able to identify and determine amino acid and/or nucleic acid sequences for the framework regions (FRs) based on the provided sequence information. In certain embodiments, the extracellular antigen-binding domain is comprises a V_{H} and/or V_{L}, sequence selected from the group consisting of SEQ ID NOs: 7 and 8, including post-translational modifications of that sequence (SEQ ID NO: 7 or 8).

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In certain embodiments, the extracellular antigen-binding domain of a presently disclosed CAR cross-competes for binding to Sialyl Lewis A (e.g., human Sialyl Lewis A) with a reference antibody or an antigen-binding portion thereof comprising the V_{H} CDR1, CDR2, and CDR3 sequences and the and V_{L} CDR1, CDR2, and CDR3 sequences of, for example, any one of the presently disclosed scFvs. In certain embodiments, the extracellular antigen-binding domain of a presently disclosed CAR cross-competes for binding to Sialyl Lewis A (e.g., human Sialyl Lewis A) with a reference antibody or an antigen-binding portion thereof comprising the V_{H} and V_{L} sequences of, for example, any one of the presently disclosed scFvs.

In certain embodiments, the extracellular antigen-binding domain of a presently disclosed CAR cross-competes for binding to Sialyl Lewis A (e.g., human Sialyl Lewis A) with a reference antibody or an antigen-binding portion thereof comprising the V_{H} CDR1, CDR2, and CDR3 sequences and the V_{L} CDR1, CDR2, and CDR3 sequences of scFv 5B1. For example, the extracellular antigen-binding domain of a presently disclosed CAR cross-competes for binding to Sialyl Lewis A (e.g., human Sialyl Lewis A) with a reference antibody or an antigen-binding portion thereof comprising a V_{H} CDR1 comprising the amino acid sequence set forth in SEQ ID NO: 1; a V_{H} CDR2 comprising the amino acid sequence set forth in SEQ ID NO: 2; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the extracellular antigen-binding domain of a presently disclosed CAR cross-competes for binding to Sialyl Lewis A with a reference antibody or an antigen-binding portion thereof comprising the V_{H} and V_{L} sequences of scFv 5B1. For example, the extracellular antigen-binding domain of a presently disclosed CAR cross-competes for binding to Sialyl Lewis A with a reference antibody or an antigen-binding portion thereof comprising a V_{H} comprising the amino acid sequence set forth in SEQ ID NO: 7, and a V_{L} comprising the amino acid sequence set forth in SEQ ID NO: 8.

In certain embodiments, the extracellular antigen-binding domain binds to the same or overlapping epitope on Sialyl Lewis A (e.g., human Sialyl Lewis A) as the reference antibody or antigen-binding portion thereof. For example, the extracellular antigen-binding domain of a presently disclosed CAR binds to the same or overlapping epitope on Sialyl Lewis A (e.g., human Sialyl Lewis A) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs. In certain embodiments, the extracellular antigen-binding domain of a presently disclosed CAR binds to the same or overlapping epitope on Sialyl Lewis A (e.g., human Sialyl Lewis A) as a reference antibody or an antigen-binding portion thereof comprising the V_{H} and V_{L} sequences of, for example, any one of the presently disclosed scFvs.

In certain embodiments, the extracellular antigen-binding domain of a presently disclosed CAR binds to the same or overlapping epitope on Sialyl Lewis A (e.g., human Sialyl Lewis A) as a reference antibody or an antigen-binding portion thereof comprising the V_{H} CDR1, CDR2, and CDR3 sequences and the V_{L} CDR1, CDR2, and CDR3 sequences of scFv 5B1. For example, the extracellular antigen-binding domain of a presently disclosed CAR binds to the same or overlapping epitope on Sialyl Lewis A (e.g., human Sialyl Lewis A) as a reference antibody or an antigen-binding portion thereof comprising a V_{H} CDR1 comprising the amino acid sequence set forth in SEQ ID NO: 1; a V_{H} CDR2 comprising the amino acid sequence set forth in SEQ ID NO: 2; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the extracellular antigen-binding domain of a presently disclosed CAR binds to the same or substantially the same epitope on Sialyl Lewis A (e.g., human Sialyl Lewis A) as a reference antibody or an antigen-binding portion thereof comprising the V_{H} and V_{L} sequences of scFv 5B1. For example, the extracellular antigen-binding domain of a presently disclosed CAR binds to the same or overlapping epitope on Sialyl Lewis A (e.g., human Sialyl Lewis A) as a reference antibody or an antigen-binding portion thereof comprising a V_{H} comprising the amino acid sequence set forth in SEQ ID NO: 7, and a V_{L} comprising the amino acid sequence set forth in SEQ ID NO: 8.

Extracellular antigen-binding domains that cross-compete or compete with the reference antibody or antigen-binding portions thereof for binding to Sialyl Lewis A (e.g., human Sialyl Lewis A) can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a Sialyl Lewis A, admixing the reference antibody with a test extracellular antigen-binding domain, measuring a second binding of the reference antibody to the Sialyl Lewis A in the presence of the test extracellular antigen-binding domain, and comparing the initial binding with the second binding of the reference antibody, wherein a decreased second binding of the reference antibody to the Sialyl Lewis A in comparison to the initial binding indicates that the test extracellular antigen-binding domain cross-competes with the reference antibody for binding to Sialyl Lewis A, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the Sialyl Lewis A is expressed in cells, e.g., in a flow cytometry test. In certain embodiments, the Sialyl Lewis A is immobilized onto a surface, including a Biacore ship (e.g., in a Biacore test), or other media suitable for surface plasmon resonance analysis. The binding of the reference antibody in the presence of a completely irrelevant antibody (that does not bind to Sialyl Lewis A) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to an Sialyl Lewis A by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to Sialyl Lewis A. In certain embodiments, the assays are performed at room temperature.

In certain embodiments, the antibody-binding assay comprises measuring an initial binding of a test extracellular antigen-binding domain to Sialyl Lewis A, admixing the test extracellular antigen-binding domain with a reference antibody, measuring a second binding of the test extracellular antigen-binding domain to the Sialyl Lewis A polypeptide in the presence of the reference antibody, and comparing the initial binding with the second binding of the test extracellular antigen-binding domain, where a decreased second binding of the test extracellular antigen-binding domain to the Sialyl Lewis A in comparison to the initial binding indicates that the test extracellular antigen-binding domain cross-competes with the reference antibody for binding to Sialyl Lewis A, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the Sialyl Lewis A is expressed in cells, e.g., in a flow cytometry test. In certain embodiments, the Sialyl Lewis A is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to Sialyl Lewis A) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to Sialyl Lewis A is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to Sialyl Lewis A. In certain embodiments, the assays are performed at room temperature.

It is well known in the art that the CDR3 domain, independently from the CDR1 and/or CDR2 domain(s), alone can determine the binding specificity of an antibody or an antigen-binding portion thereof, for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Bioi. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad Sci. U.S.A. 95:8910-8915 (1998) (describing a panel of humanized anti-integrin 43 antibodies using a heavy and light chain variable CDR3 domain of a murine anti-integrin αᵥβ₃ antibody LM609 wherein each member antibody comprises a distinct sequence outside the CDR3 domain and capable of binding the same epitope as the parent muring antibody with affinities as high or higher than the parent murine antibody); Barbas et al., J. Am. Chem. Soc. 116:2161-2162 (1994) (disclosing that the CDR3 domain provides the most significant contribution to antigen binding); Barbas et al., Proc. Natl. Acad Sci. U.S.A. 92:2529-2533 (1995) (describing the grafting of heavy chain CDR3 sequences of three Fabs (SI-1, SI-40, and SI-32) against human placental DNA onto the heavy chain of an anti-tetanus toxoid Fab thereby replacing the existing heavy chain CDR3 and demonstrating that the CDR3 domain alone conferred binding specificity); and Ditzel et al., J. Immunol. 157:739-749 (1996) (describing grafting studies wherein transfer of only the heavy chain CDR3 of a parent polyspecific Fab LNA3 to a heavy chain of a monospecific IgG tetanus toxoid-binding Fab p313 antibody was sufficient to retain binding specificity of the parent Fab). Each of these references is hereby incorporated by reference in its entirety.

In certain embodiments, the extracellular antigen-binding domain of a CAR described herein comprises a heavy chain variable region CDR3 comprising the amino acid sequence set forth in SEQ ID NO: 3, a conservative modification of SEQ ID NO: 3, and/or a light chain variable region CDR3 comprising the amino acid sequence set forth in SEQ ID NO: 6 or a conservative modification thereof. In some such embodiments, the extracellular antigen-binding domain can also (i) comprise a heavy chain variable region CDR2 comprising the amino acid sequence set forth in SEQ ID NO: 2 or a conservative modification thereof, and a light chain variable region CDR2 comprising the amino acid sequence set forth in SEQ ID NO: 5 or a conservative modification thereof; and/or (ii) comprise a heavy chain variable region CDR1 comprising the amino acid sequence set forth in SEQ ID NO: 1 or a conservative modification thereof, and a light chain variable region CDR1 comprising the amino acid sequence set forth in SEQ ID NO: 4 or a conservative modification thereof.

In certain embodiments, the extracellular antigen-binding domain comprises a V_{H} CDR1 comprising the amino acid sequence set forth in SEQ ID NO: 1, a V_{H} CDR2 comprising the amino acid sequence set forth in SEQ ID NO: 2, a V_{H} CDR3 comprising the amino acid sequence set forth in SEQ ID NO: 3, a V_{L} CDR1 comprising the amino acid sequence set forth in SEQ ID NO: 4, a V_{L} CDR2 comprising the amino acid sequence set forth in SEQ ID NO: 5, and a V_{L} CDR3 comprising the amino acid sequence set forth in SEQ ID NO: 6.

Furthermore, in certain embodiments, the extracellular antigen-binding domain comprises a heavy chain variable region CDR3 comprising the amino acid sequence set forth in SEQ ID NO: 3 or a conservative modification thereof; and a light chain variable region CDR3 comprising the amino acid sequence set forth in SEQ ID NO: 6 or a conservative modification thereof.

In certain embodiments, the extracellular antigen-binding domain comprises a heavy chain variable region CDR3 comprising the amino acid sequence set forth in SEQ ID NO: 3 or a conservative modification thereof; and a light chain variable region CDR3 comprising the amino acid sequence set forth in SEQ ID NO: 6 or a conservative modification thereof.

The extracellular antigen-binding domain can further comprise a heavy chain variable region CDR2 comprising the amino acid sequence set forth in SEQ ID NO: 2 or a conservative modification thereof; and a light chain variable region CDR2 comprising the amino acid sequence set forth in SEQ ID NO: 5 or a conservative modification thereof.

In certain embodiments, the extracellular antigen-binding domain comprises a heavy chain variable region CDR2 comprising the amino acid sequence set forth in SEQ ID NO: 2 or a conservative modification thereof; and a light chain variable region CDR2 comprising the amino acid sequence set forth in SEQ ID NO: 5 or a conservative modification thereof.

The extracellular antigen-binding domain can further comprise a heavy chain variable region CDR1 comprising the amino acid sequence set forth in SEQ ID NO: 1 or a conservative modification thereof; and a light chain variable region CDR1 comprising the amino acid sequence set forth in SEQ ID NO: 4 or a conservative modification thereof.

In certain embodiments, the extracellular antigen-binding domain comprises a heavy chain variable region CDR1 comprising the amino acid sequence set forth in SEQ ID NO: 1 or a conservative modification thereof; and a light chain variable region CDR1 comprising the amino acid sequence set forth in SEQ ID NO: 4 or a conservative modification thereof.

In certain non-limiting embodiments, an extracellular antigen-binding domain of the presently disclosed CAR can comprise a linker connecting the heavy chain variable region and light chain variable region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 11. In certain embodiments, the nucleotide sequence encoding the amino acid sequence of SEQ ID NO: 11 is set forth in SEQ ID NO: 12.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide comprising the amino acid sequence set forth in SEQ ID NO: 13 as provided below.

[SEQ ID NO: 13]
TAMALPVTALLLPLALLLHAARP

An exemplary nucleotide sequence encoding the amino acid sequence of SEQ ID NO: 13 is set forth in SEQ ID NO: 14, which is provided below:

[SEQ ID NO: 14]
ACTGCCATGGCCCTGCCAGTAACGGCTCTGCTGCTGCCACTTGCTCTGCT
CCTCCATGCAGCCAGGCCT

Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 polypeptide, a NKGD2 polypeptide, a synthetic polypeptide (not based on a protein associated with the immune response), or a combination thereof.

In certain embodiments, the transmembrane domain of a presently disclosed CAR comprises a CD28 polypeptide. In certain embodiments, the transmembrane domain of a presently disclosed CAR comprises a human CD28 polypeptide (e.g., a transmembrane domain of human CD28 or a portion thereof). The CD28 polypeptide can have an amino acid sequence that is at least about 85%, about 90%, about 95%, about 96%, about 97%, about 98%, about 99% or 100% homologous (e.g., identical) to the sequence having a NCBI Reference No: P10747 or NP_006130 (SEQ ID No:15) or a fragment thereof, and/or may optionally comprise up to one or up to two or up to three conservative amino acid substitutions. In certain embodiments, the CD28 polypeptide can have an amino acid sequence that is a consecutive portion of SEQ ID NO: 15 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 15. In certain embodiments, the CAR of the presently disclosed comprises a transmembrane domain comprising a CD28 polypeptide, and an intracellular domain comprising a co-stimulatory signaling region that comprises a CD28 polypeptide. In certain embodiments, the CD28 polypeptide comprised in the transmembrane domain and the intracellular domain comprises or has amino acids 114 to 220 of SEQ ID NO: 15.

SEQ ID NO: 15 is provided below:

[SEQ ID NO: 15]

| | |
|---|---|
| 1 | MLRLLLALNL FPSIQVTGNK ILVKQSPMLV AYDNAVNLSC |
| | KYSYNLFSRE FRASLHKGLD |
| 61 | SAVEVCVVYG NYSQQLQVYS KTGFNCDGKL GNESVTFYLQ |
| | NLYVNQTDIY FCKIEVMYPP |
| 121 | PYLDNEKSNG TIIHVKGKHL CPSPLFPGPS KPFWVLVVVG |
| | GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide. An exemplary nucleotide sequence encoding amino acids 114 to 220 of SEQ ID NO: 15 is set forth in SEQ ID NO: 16, which is provided below.

[SEQ ID NO: 16]
attgaagttatgtatcctcctccttacctagacaatgagaagagcaatgg
aaccattatccatgtgaaagggaaacacctttgtccaagtcccctatttc
ccggaccttctaagcccttttgggtgctggtggtggttggtggagtcctg
gcttgctatagcttgctagtaacagtggcctttattattttctgggtgag
gagtaagaggagcaggctcctgcacagtgactacatgaacatgactcccc
gccgccccgggcccacccgcaagcattaccagccctatgccccaccacgc
gacttcgcagcctatcgctcc

In certain embodiments, the transmembrane domain comprises a CD8 polypeptide (e.g., a transmembrane domain of CD28 or a portion thereof). The CD8 polypeptide can have an amino acid sequence that is at least about 85%, about 90%, about 95%, about 96%, about 97%, about 98%, about 99% or about 100% homologous to SEQ ID NO: 17 or a fragment thereof, and/or may optionally comprise up to one or up to two or up to three conservative amino acid substitutions. In certain embodiments, the CD8 polypeptide can have an amino acid sequence that is a consecutive portion of SEQ ID NO: 17 which is at least 20, or at least 30, or at least 40, or at least 50, and up to 235 amino acids in length. Alternatively or additionally, in non-limiting various embodiments, the CD8 polypeptide comprises or has amino acids 1 to 235, 1 to 50, 50 to 100, 100 to 150, 150 to 200, or 200 to 235 of SEQ ID NO: 17.

[SEQ ID NO: 17]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In accordance with the presently disclosed subject matter, a “CD8 nucleic acid molecule” refers to a polynucleotide encoding a CD8 polypeptide.

In certain embodiments, the transmembrane domain of a presently disclosed CAR comprises a native or modified transmembrane domain of a CD166 polypeptide. The CD166 polypeptide can have an amino acid sequence that is at least about 85%, about 90%, about 95%, about 96%, about 97%, about 98%, about 99% or 100% homologous to the sequence having a NCBI Reference No: NP_001618.2 (SEQ ID No: 18), or fragments thereof, and/or may optionally comprise up to one or up to two or up to three conservative amino acid substitutions. In non-limiting certain embodiments, the CD166 polypeptide comprises or has an amino acid sequence that is a consecutive portion of SEQ ID NO: 18 which is at least 20, or at least 30, or at least 40, or at least 50, and up to 583 amino acids in length. Alternatively or additionally, in non-limiting various embodiments, the CD166 polypeptide comprises or has an amino acid sequence of amino acids 1 to 583, 1 to 50, 50 to 100, 100 to 150, 150 to 200, 150 to 200, 200 to 250, 250 to 300, 300 to 350, 350 to 400, 400 to 450, 450 to 500, 528 to 553, 500 to 550, or 550 to 583 of SEQ ID NO: 18. In certain embodiments, the CD166 polypeptide comprised in the transmembrane domain of a presently disclosed CAR comprises or has amino acids 528 to 553 of SEQ ID NO: 18.

SEQ ID NO: 18 is provided below:

[SEQ ID NO: 18]
MESKGASSCRLLFCLLISATVFRPGLGWYTVNSAYGDTIIIPCRLDVPQN
LMFGKWKYEKPDGSPVFIAFRSSTKKSVQYDDVPEYKDRLNLSENYTLSI
SNARISDEKREVCMLVTEDNVFEAPTIVKVEKQPSKPEIVSKALFLETEQ
LKKLGDCISEDSYPDGNITWYRNGKVLHPLEGAVVIIFKKEMDPVTQLYT
MTSTLEYKTTKADIQMPFTCSVTYYGPSGQKTIHSEQAVEDIYYPTEQVT
IQVLPPKNAIKEGDNITLKCLGNGNPPPEEFLFYLPGQPEGIRSSNTYTL
TDVRRNATGDYKCSLIDKKSMIASTAITVHYLDLSLNPSGEVTRQIGDAL
PVSCTISASRNATVVWMKDNIRLRSSPSFSSLHYQDAGNYVCETALQEVE
GLKKRESLTLIVEGKPQIKMTKKTDPSGLSKTIICHVEGFPKPAIQWTIT
GSGSVINQTEESPYINGRYYSKIIISPEENVTLTCTAENQLERTVNSLNV
SAISIPEHDEADEISDENREKVNDQAKLIVGIVVGLLLAALVAGVVYWLY
MKKSKTASKHVNKDLGNMEENKKLEENNHKTEA

In accordance with the presently disclosed subject matter, a “CD166 nucleic acid molecule” refers to a polynucleotide encoding a CD166 polypeptide. An exemplary nucleotide sequence encoding amino acids 528 to 553 of SEQ ID NO: 18 is set forth in SEQ ID NO: 19, which is provided below.

[SEQ ID NO: 19]
CTAATTGTGGGAATCGTTGTTGGTCTCCTCCTTGCTGCCCTTGTTGCTGG
TGTCGTCTACTGGCTGTACATGAAGAAG

Hinge/Spacer Region

In certain non-limiting embodiments, a CAR can also comprise a hinge/spacer region that links the extracellular antigen-binding domain to the transmembrane domain. A hinge/spacer region can be flexible enough to allow the antigen-binding domain to orient in different directions to facilitate antigen recognition while preserving the activating activity of the CAR. In certain non-limiting embodiments, the hinge/spacer region can be the hinge region from IgG1, the CH₂CH₃ region of immunoglobulin and portions of CD3, a portion of a CD28 polypeptide (e.g., SEQ ID NO:15), a portion of a CD8 polypeptide (e.g., SEQ ID NO: 17), a portion of a CD166 polypeptide (e.g., SEQ ID NO: 18), a variation of any of the foregoing which is at least about 80%, at least about 85%, at least about 90%, or at least about 95% homologous thereto, or a synthetic spacer sequence. In certain non-limiting embodiments, the hinge/spacer region may have a length between about 1-50 (e.g., 5-25, 10-30, or 30-50) amino acids.

In certain embodiments, the hinge/spacer region of a presently disclosed CAR comprises a native or modified (e.g., with a conservative modification) hinge region of a CD166 polypeptide as described herein. In certain embodiments, the CD166 polypeptide comprised in the hinge/spacer region of a presently disclosed CAR comprises or has an amino acid sequence of amino acids 489 to 527 of SEQ ID NO:18. An exemplary nucleotide sequence encoding amino acids 489 to 527 of SEQ ID NO: 18 is set forth in SEQ ID NO: 20, which is provided below.

[SEQ ID NO: 20]
ACCAACTGGAGAGAACAGTAAACTCCTTGAATGTCTCTGCTATAAGTATT
CCAGAACACGATGAGGCAGACGAGATAAGTGATGAAAACAGAGAAAAGGT
GAATGACCAGGCAAAA

Intracellular Domain of a CAR

In certain non-limiting embodiments, the intracellular signaling domain of a CAR described herein comprises a CD3ζ polypeptide, which can activate or stimulate a cell (e.g., a cell of the lymphoid lineage, e.g., a T cell). Wild type (“native”) CD3ζ comprises three immunoreceptor tyrosine-based activation motifs (“ITAMs”) (e.g., ITAM1, ITAM2 and ITAM3), three basic-rich stretch (BRS) regions (BRS1, BRS2 and BRS3), and transmits an activation signal to the cell (e.g., a cell of the lymphoid lineage, e.g., a T cell) after antigen is bound. The intracellular signaling domain of the native CD3ζ-chain is the primary transmitter of signals from endogenous TCRs. CD3ζ, as used in embodiments herein, is not native CD3ζ but is a modified CD3ζ. In certain embodiments, the intracellular signaling domain of a presently disclosed CAR comprises a CD3ζ polypeptide disclosed in International Patent Application No.: PCT/US2018/068134 filed Dec. 31, 2018 (corresponding to International Publication Number WO 2019/133969), the content of which is herein incorporated by reference in its entirety for the purposes described herein.

In certain embodiments, the modified CD3ζ polypeptide comprises or has an amino acid sequence that is at least about 85%, about 90%, about 95%, about 96%, about 97%, about 98%, or about 99% homologous to the sequence having a NCBI Reference No: NP_932170 (SEQ ID No: 21), or a fragment thereof. In certain non-limiting embodiments, the modified CD3ζ polypeptide comprises or has an amino acid sequence that is a consecutive portion of SEQ ID NO: 21, which is at least 20, or at least 30, or at least 40, or at least 50, or at least 100, or at least 110, or at least 113, and up to 163 amino acids in length. Alternatively or additionally, in non-limiting various embodiments, the modified CD3ζ polypeptide comprises or has an amino acid sequence of amino acids 1 to 50, 50 to 100, 100 to 150, 50 to 164, 55 to 164, or 150 to 164 of SEQ ID NO: 21. In certain embodiments, the modified CD3ζ polypeptide comprises or has an amino acid sequence of amino acids 52 to 164 of SEQ ID NO: 21.

SEQ ID NO: 21 is provided below:

[SEQ ID NO: 21]

| | |
|---|---|
| 1 | MKWKALFTAA ILQAQLPITE AQSFGLLDPK LCYLLDGILF |
| | IYGVILTALF LRVKFSRSAD |
| 61 | APAYQQGQNQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

In certain embodiments, the intracellular signaling domain of the CAR comprises a modified human CD3ζ polypeptide. The modified human CD3ζ polypeptide can comprise or have an amino acid sequence that is at least about 85%, about 90%, about 95%, about 96%, about 97%, about 98%, or about 99% or about 100% homologous (e.g., identical) to SEQ ID NO: 22 or a fragment thereof, and/or may optionally comprise up to one or up to two or up to three conservative amino acid substitutions. SEQ ID NO: 22 is provided below:

[SEQ ID NO: 22]
RVKFSRSADA PAYQQGQNQL YNELNLGRRE EYDVLDKRRG
RDPEMGGKPR RKNPQEGLYN ELQKDKMAEA YSEIGMKGER
RRGKGHDGLY QGLSTATKDT YDALHMQALP PR.

An exemplary nucleic acid sequence encoding the amino acid sequence of SEQ ID NO: 22 is set forth in SEQ ID NO: 23, which is provided below.

[SEQ ID NO: 23]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In certain embodiments, the intracellular signaling domain of the CAR comprises a modified CD3ζ polypeptide. In certain embodiments, the intracellular signaling domain of the CAR comprises a modified human CD3ζ polypeptide. In certain embodiments, the modified CD3ζ polypeptide comprises or has an amino acid sequence that is at least about 80%, at least about 85%, at least about 90%, at least about 95%, at least about 96%, at least about 97%, at least about 98%, or at least about 99%, at least about 100% homologous (e.g., identical) to SEQ ID NO: 24 or a fragment thereof, and/or may optionally comprise up to one or up to two or up to three conservative amino acid substitutions. SEQ ID NO: 24 is provided below:

[SEQ ID NO: 24]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

An exemplary nucleic acid sequence encoding the amino acid sequence of SEQ ID NO: 24 is set forth in SEQ ID NO: 25, which is provided below.

[SEQ ID NO: 25]
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtTcaatgaactgcagaaagataagat
ggcggaggcctTcagtgagattgggatgaaaggcgagcgccggaggggca
aggggcacgatggcctttTccaggggctcagtacagccaccaaggacacc
tTcgacgcccttcacatgcaggccctgccccctcgc

Immunoreceptor tyrosine-based activation motifs (ITAMs) In certain non-limiting embodiments, the intracellular signaling domain of the CAR comprises a modified CD3ζ polypeptide comprising one, two or three ITAMs. In certain embodiments, the modified CD3ζ polypeptide comprises a native ITAM1 comprising the amino acid sequence set forth in SEQ ID NO: 26.

[SEQ ID NO: 26]
QNQLYNELNLGRREEYDVLDKR

An exemplary nucleic acid sequence encoding the amino acid sequence of SEQ ID NO: 26 is set forth in SEQ ID NO: 27, which is provided below.

[SEQ ID NO: 27]
cagaaccagctctataacgagctcaatctagga cgaagagaggagtacg
atgttttggacaagaga

In certain embodiments, the modified CD3ζ polypeptide comprises an ITAM1 variant comprising one or more loss-of-function mutations. In certain embodiments, the modified CD3ζ polypeptide has an ITAM1 variant comprising two loss-of-function mutations. In certain embodiments, the loss of function mutation comprises a mutation of a tyrosine residue in ITAM1. In certain embodiments, the ITAM1 variant consisting of two loss-of-function mutations comprises the amino acid sequence set forth in SEQ ID NO: 28, which is provided below.

[SEQ ID NO: 28]
QNQLFNELNLGRREEFDVLDKR

An exemplary nucleic acid sequence encoding the amino acid sequence of SEQ ID NO: 28 is set forth in SEQ ID NO: 29, which is provided below.

[SEQ ID NO: 29]
cagaaccagctctTtaacgagctcaatctagga cgaagagaggagtTcg
atgttttggacaagaga

In certain embodiments, the modified CD3ζ polypeptide comprises a native ITAM2 comprising the amino acid sequence set forth in SEQ ID NO: 30, which is provided below.

[SEQ ID NO: 30]
QEGLYNELQKDKMAEAYSEIGMK

An exemplary nucleic acid sequence encoding the amino acid sequence of SEQ ID NO: 30 is set forth in SEQ ID NO: 31, which is provided below.

[SEQ ID NO: 31]
caggaaggcctgtacaatgaactgcagaaagataagatggcggaggccta
cagtgagattgggatgaaa

In certain embodiments, the modified CD3ζ polypeptide comprises an ITAM2 variant comprising one or more loss-of-function mutations. In certain embodiments, the modified CD3ζ polypeptide has an ITAM2 variant comprising two loss-of-function mutations. In certain embodiments, the loss of function mutation comprises a mutation of a tyrosine residue in ITAM2. In certain embodiments, the ITAM2 variant consisting of two loss-of-function mutations comprises the amino acid sequence set forth in SEQ ID NO: 32, which is provided below.

[SEQ ID NO: 32]
QEGLFNELQKDKMAEAFSEIGMK

An exemplary nucleic acid sequence encoding the amino acid sequence of SEQ ID NO: 32 is set forth in SEQ ID NO: 33, which is provided below.

[SEQ ID NO: 33]
caggaaggcctgtTcaatgaactgcagaaagataagatggcggaggcctT
cagtgagattgggatgaaa

In certain embodiments, the modified CD3ζ polypeptide comprises a native ITAM3 comprising the amino acid sequence set forth in SEQ ID NO: 34, which is provided below.

[SEQ ID NO: 34]
HDGLYQGLSTATKDTYDALHMQ

An exemplary nucleic acid sequence encoding the amino acid sequence of SEQ ID NO: 34 is set forth in SEQ ID NO: 35, which is provided below.

[SEQ ID NO: 35]
cacgatggcctttaccagggtctcagtacagccaccaaggacacctacga
cgcccttcacatgcag

In certain embodiments, the modified CD3ζ polypeptide comprises an ITAM3 variant comprising one or more loss-of-function mutations. In certain embodiments, the modified CD3ζ polypeptide has an ITAM3 variant comprising two loss-of-function mutations. In certain embodiments, the loss of function mutation comprises a mutation of a tyrosine residue in ITAM3. In certain embodiments, the ITAM3 variant consisting of two loss-of-function mutations comprises the amino acid sequence set forth in SEQ ID NO: 36, which is provided below.

[SEQ ID NO: 36]
HDGLFQGLSTATKDTFDALHMQ

An exemplary nucleic acid sequence encoding the amino acid sequence of SEQ ID NO: 36 is set forth in SEQ ID NO: 37, which is provided below.

[SEQ ID NO: 37]
cacgatggcctttTccaggggctcagtacagccaccaaggacacctTcga
cgcccttcacatgcag

In certain embodiments, the intracellular signaling domain of the CAR comprises a modified CD3ζ polypeptide comprising or consisting essentially of or consisting of an ITAM1 variant comprising one or more loss-of-function mutations, an ITAM2 variant comprising one or more loss-of-function mutations, an ITAM3 variant comprising one or more loss-of-function mutations, or a combination thereof. In certain embodiments, the intracellular signaling domain of the CAR comprises a modified CD3ζ polypeptide comprising an ITAM2 variant comprising one or more (e.g., two) loss-of-function mutations and an ITAM3 variant comprising one or more (e.g., two) loss-of-function mutations. In certain embodiments, the intracellular signaling domain of the CAR comprises a modified CD3ζ polypeptide comprising a native ITAM1, an ITAM2 variant comprising or having two loss-of-function mutations and an ITAM3 variant comprising or having two loss-of-function mutations. In certain embodiments, the intracellular signaling domain of the CAR comprises a modified CD3ζ polypeptide comprising a native ITAM1 having the amino acid sequence set forth in SEQ ID NO: 26, an ITAM2 variant having the amino acid sequence set forth in SEQ ID NO: 32, and an ITAM3 variant having the amino acid sequence set forth in SEQ ID NO: 36. In certain embodiments, the modified CD3ζ polypeptide comprising or has the amino acid sequence set forth in SEQ ID NO: 24.

In certain embodiments, the intracellular signaling domain of the CAR comprises a modified CD3ζ polypeptide comprising an ITAM1 variant comprising one or more (e.g., two) loss-of-function mutations and an ITAM3 variant comprising one or more (e.g., two) loss-of-function mutations. In certain embodiments, the intracellular signaling domain of the CAR comprises a modified CD3ζ polypeptide comprising an ITAM1 variant comprising two loss-of-function mutations, a native ITAM2, and an ITAM3 variant comprising two loss-of-function mutations. In certain embodiments, the intracellular signaling domain of the CAR comprises a modified CD3ζ polypeptide comprising an ITAM1 variant having the amino acid sequence set forth in SEQ ID NO: 28, a native ITAM2 having the amino acid sequence set forth in SEQ ID NO: 30, and an ITAM3 variant having the amino acid sequence set forth in SEQ ID NO: 36.

In certain embodiments, the intracellular signaling domain of the CAR comprises a modified CD3ζ polypeptide comprising an ITAM1 variant comprising one or more (e.g., two) loss-of-function mutations and an ITAM2 variant comprising one or more (e.g., two) loss-of-function mutations. In certain embodiments, the intracellular signaling domain of the CAR comprises a modified CD3ζ polypeptide comprising an ITAM1 variant comprising two loss-of-function mutations, an ITAM2 variant comprising two loss-of-function mutations, and a native ITAM3. In certain embodiments, the intracellular signaling domain of the CAR comprises a modified CD3ζ polypeptide comprising an ITAM1 variant having the amino acid sequence set forth in SEQ ID NO: 28, an ITAM2 variant having the amino acid sequence set forth in SEQ ID NO: 32, and a native ITAM3 having the amino acid sequence set forth in SEQ ID NO: 34.

In certain embodiments, the intracellular signaling domain of the CAR comprises a modified CD3ζ polypeptide comprising an ITAM1 variant comprising one or more (e.g., two) loss-of-function mutations. In certain embodiments, the intracellular signaling domain of the CAR comprises a modified CD3ζ polypeptide comprising an ITAM1 variant comprising two loss-of-function mutations, a native ITAM2, and a native ITAM3. In certain embodiments, the intracellular signaling domain of the CAR comprises a modified CD3ζ polypeptide comprising an ITAM1 variant having the amino acid sequence set forth in SEQ ID NO: 28, a native ITAM2 having the amino acid sequence set forth in SEQ ID NO: 30, and a native ITAM3 having the amino acid sequence set forth in SEQ ID NO: 34.

In certain embodiments, the intracellular signaling domain of the CAR comprises a modified CD3ζ polypeptide comprising a native ITAM1, a native ITAM2, and an ITAM3 variant comprising one or more (e.g., two) loss-of-function mutations. In certain embodiments, the intracellular signaling domain of the CAR comprises a modified CD3ζ polypeptide comprising a native ITAM1, a native ITAM2, and an ITAM1 variant comprising two loss-of-function mutations. In certain embodiments, the intracellular signaling domain of the CAR comprises a modified CD3ζ polypeptide comprising a native ITAM1 having the amino acid sequence set forth in SEQ ID NO: 26, a native ITAM2 having the amino acid sequence set forth in SEQ ID NO: 30, and an ITAM3 variant having the amino acid sequence set forth in SEQ ID NO: 36.

In certain embodiments, the intracellular signaling domain of the CAR comprises a modified CD3ζ polypeptide comprising a native ITAM1, an ITAM2 variant comprising one or more (e.g., two) loss-of-function mutations, and a native ITAM3. In certain embodiments, the intracellular signaling domain of the CAR comprises a modified CD3ζ polypeptide comprising a native ITAM1, an ITAM2 variant comprising two loss-of-function mutations, and a native ITAM3. In certain embodiments, the intracellular signaling domain of the CAR comprises a modified CD3ζ polypeptide comprising a native ITAM1 having the amino acid sequence set forth in SEQ ID NO: 26, an ITAM2 variant having the amino acid sequence set forth in SEQ ID NO: 32, and a native ITAM3 having the amino acid sequence set forth in SEQ ID NO: 34.

In certain embodiments, the intracellular signaling domain of the CAR comprises a modified CD3ζ polypeptide comprising a deletion of one or two ITAMs. In certain embodiments, the modified CD3ζ polypeptide comprises a deletion of ITAM1 and ITAM2, e.g., the modified CD3ζ polypeptide comprises a native ITAM3 or a ITAM3 variant, and does not comprise an ITAM1 or an ITAM2. In certain embodiments, the modified CD3ζ polypeptide comprises a native ITAM3 having the amino acid sequence set forth in SEQ ID NO: 34, and does not comprise an ITAM1 (native or modified), or an ITAM2 (native or modified).

In certain embodiments, the modified CD3ζ polypeptide comprises a deletion of ITAM2 and ITAM3, e.g., the modified CD3ζ polypeptide comprises a native ITAM1 or a ITAM1 variant, and does not comprise an ITAM2 or an ITAM3. In certain embodiments, the modified CD3ζ polypeptide comprises a native ITAM1 having the amino acid sequence set forth in SEQ ID NO: 26, and does not comprise an ITAM2 (native or modified), or an ITAM3 (native or modified).

In certain embodiments, the modified CD3ζ polypeptide comprises a deletion of ITAM1 and ITAM3, e.g., the modified CD3ζ polypeptide comprises a native ITAM2 or a ITAM2 variant, and does not comprise an ITAM1 or an ITAM3. In certain embodiments, the modified CD3ζ polypeptide comprises a native ITAM2 having the amino acid sequence set forth in SEQ ID NO: 30, and does not comprise an ITAM1 (native or modified), or an ITAM3 (native or modified).

In certain embodiments, the modified CD3ζ polypeptide comprises a deletion of ITAM1, e.g., the modified CD3ζ polypeptide comprises a native ITAM2 or an ITAM2 variant, and a native ITAM3 or an ITAM3 variant, and does not comprise an ITAM1 (native or modified). In certain embodiments, the modified CD3ζ polypeptide comprises a deletion of ITAM2, e.g., the modified CD3ζ polypeptide comprises a native ITAM1 or an ITAM1 variant, and a native ITAM3 or an ITAM3 variant, and does not comprise an ITAM2 (native or modified). In certain embodiments, the modified CD3ζ polypeptide comprises a deletion of ITAM3, e.g., the modified CD3ζ polypeptide comprises a native ITAM1 or an ITAM1 variant, and a native ITAM2 or an ITAM2 variant, and does not comprise an ITAM3 (native or modified).

Basic-Rich Stretch (BRS) Region

In certain non-limiting embodiments, the intracellular signaling domain of the CAR comprises a modified CD3ζ polypeptide comprising one, two or three BRS regions (i.e., BRS1, BRS2, and BRS3). The BRS region can be a native BRS or a modified BRS (e.g., a BRS variant). In certain embodiments, the modified CD3ζ polypeptide comprises a native BRS1 region comprising the amino acid sequence set forth in SEQ ID NO: 38, which is provided below.

[SEQ ID NO: 38]
KRRGR

An exemplary nucleic acid sequence encoding the amino acid sequence of SEQ ID NO: 38 is set forth in SEQ ID NO: 39, which is provided below.

[SEQ ID NO: 39]
aagagacgtggccgg

In certain embodiments, the modified CD3ζ polypeptide comprises a BRS1 variant comprising one or more loss-of-function mutations.

In certain embodiments, the modified CD3ζ polypeptide comprises a native BRS2 comprising the amino acid sequence set forth in SEQ ID NO: 40.

[SEQ ID NO: 40]
KPRRK

An exemplary nucleic acid sequence encoding the amino acid sequence of SEQ ID NO: 40 is set forth in SEQ ID NO: 41, which is provided below.

[SEQ ID NO: 41]
aagccgagaaggaag

In certain embodiments, the modified CD3ζ polypeptide comprises a BRS2 variant comprising one or more loss-of-function mutations.

In certain embodiments, the modified CD3ζ polypeptide comprises a native BRS3 comprising the amino acid sequence set forth in SEQ ID NO: 42.

[SEQ ID NO: 42]
KGERRRGK

An exemplary nucleic acid sequence encoding the amino acid sequence of SEQ ID NO: 42 is set forth in SEQ ID NO: 43, which is provided below.

[SEQ ID NO: 43]
aaaggcgagcgccggaggggcaag

In certain embodiments, the modified CD3ζ polypeptide comprises a BRS3 variant comprising one or more loss-of-function mutations.

In certain embodiments, the intracellular signaling domain of the CAR comprises a modified CD3ζ polypeptide comprising all three BRS regions, i.e., a BRS1 region, a BRS2 region, and a BRS3 region. In certain embodiments, the intracellular signaling domain of the CAR comprises a modified CD3ζ polypeptide comprising a native BRS1, a native BRS2, and a native BRS3.

In certain embodiments, the intracellular signaling domain of the CAR comprises a modified CD3ζ polypeptide comprising one or two but not all three BRS regions. In certain embodiments, the modified CD3ζ polypeptide comprises a BRS1 region and a BRS2 region, and does not comprise a BRS3 region. In certain embodiments, the modified CD3ζ polypeptide comprises a BRS1 region and a BRS3 region, and does not comprise a BRS2 region. In certain embodiments, the modified CD3ζ polypeptide comprises a BRS2 region and a BRS3 region, and does not comprise a BRS1 region.

In certain embodiments, the modified CD3ζ polypeptide comprises a BRS1 region, and does not comprise a BRS2 region or a BRS3 region. In certain embodiments, the modified CD3ζ polypeptide comprises a BRS2 region, and does not comprise a BRS1 region or BRS3 region. In certain embodiments, the modified CD3ζ polypeptide comprises a BRS3 region, and does not comprise a BRS1 region or a BRS2 region.

In certain embodiments, the modified CD3ζ polypeptide does not comprise a BRS region (native or modified BRS1, BRS2 or BRS3), e.g., all three BRSs are deleted, e.g., the modified CD3ζ polypeptide comprised in construct D12.

In certain non-limiting embodiments, the CAR comprises an extracellular antigen-binding domain, a transmembrane domain, and an intracellular signaling domain comprising a modified CD3ζ polypeptide, wherein the modified CD3ζ polypeptide lacks all or part of immunoreceptor tyrosine-based activation motifs (ITAMs), wherein the ITAMs are ITAM1, ITAM2, and ITAM3. In certain embodiments, the modified CD3ζ polypeptide lacks ITAM2 or a portion thereof. In certain embodiments, the modified CD3ζ polypeptide further lacks ITAM3 or a portion thereof. In certain embodiments, the modified CD3ζ polypeptide further lacks ITAM1 or a portion thereof. In certain embodiments, the modified CD3ζ polypeptide lacks ITAM1 or a portion thereof. In certain embodiments, the modified CD3ζ polypeptide further lacks ITAM3 or a portion thereof. In certain embodiments, the modified CD3ζ polypeptide lacks ITAM3 or a portion thereof. In certain embodiments, the modified CD3ζ polypeptide lacks all or part of basic-rich stretch (BRS) regions, wherein the BRS regions are BRS1, BRS2, and BRS3. In certain embodiments, the modified CD3ζ polypeptide lacks BRS2 or a portion thereof. In certain embodiments, the modified CD3ζ polypeptide further lacks BRS3 or a portion thereof. In certain embodiments, the modified CD3ζ polypeptide further lacks BRS1 or a portion thereof. In certain embodiments, the modified CD3ζ polypeptide lacks BRS1 or a portion thereof. In certain embodiments, the modified CD3ζ polypeptide further lacks BRS3 or a portion thereof. In certain embodiments, the modified CD3ζ polypeptide lacks BRS3 or a portion thereof. In certain embodiments, the modified CD3ζ polypeptide lacks BRS1 or portion thereof, BRS2 or portion thereof, and BRS3 or a portion thereof. In certain embodiments, the modified CD3ζ polypeptide lacks ITAM2, ITAM3, BRS2, and BRS3. In certain embodiments, the CAR comprises an extracellular antigen-binding domain, a transmembrane domain, and an intracellular signaling domain comprising a modified CD3ζ polypeptide, wherein the modified CD3ζ polypeptide lacks all or part of basic-rich stretch (BRS) regions, wherein the BRS regions are BRS1, BRS2, and BRS3. In certain embodiments, the CAR comprises an extracellular antigen-binding domain, a transmembrane domain, and an intracellular signaling domain comprising a modified CD3ζ polypeptide, wherein the modified CD3ζ polypeptide comprises a BRS variant selected from a BRS1 variant, a BRS2 variant, and a BRS3 variant, wherein the BRS variant comprises one or more loss-of-function mutations.

Costimulatory Domain

In certain non-limiting embodiments, the intracellular domain of the CAR further comprises at least one co-stimulatory signaling region. In certain embodiments, the co-stimulatory signaling region comprises at least one co-stimulatory molecule or a portion thereof, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide (e.g., an intracellular domain of CD28 or a portion thereof), a 4-1BB polypeptide (e.g., an intracellular domain of 4-1BB or a portion thereof), an OX40 polypeptide (e.g., an intracellular domain of OX40 or a portion thereof), an ICOS polypeptide (e.g., an intracellular domain of ICOS or a portion thereof), a DAP-10 polypeptide (e.g., an intracellular domain of DAP-10 or a portion thereof), or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190 (e.g., the nucleotide sequence encoding 4-1BB is set forth in SEQ ID NO:15, the nucleotide sequence encoding ICOS is set forth in SEQ ID NO:16, and the nucleotide sequence encoding DAP-10 is set forth in SEQ ID NO:17 in U.S. Pat. No. 7,446,190), which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous (e.g., identical) to the sequence having a NCBI Reference No: P41273 or NP_001552 (SEQ ID NO: 44) or a fragment thereof, and/or may optionally comprise up to one or up to two or up to three conservative amino acid substitutions.

SEQ ID NO: 44 is provided below:

[SEQ ID NO: 44]

| | |
|---|---|
| 1 | MGNSCYNIVA TLLLVLNFER TRSLQDPCSN |
| | CPAGTFCDNN RNQICSPCPP NSFSSAGGQR |
| 61 | TCDICRQCKG VFRTRKECSS TSNAECDCTP |
| | GFHCLGAGCS MCEQDCKQGQ ELTKKGCKDC |
| 121 | CFGTFNDQKR GICRPWTNCS LDGKSVLVNG |
| | TKERDVVCGP SPADLSPGAS SVTPPAPARE |
| 181 | PGHSPQIISF FLALTSTALL FLLFFLTLRF |
| | SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

An OX40 polypeptide can have an amino acid sequence that is at least about 85%, about 90%, about 95%, about 96%, about 97%, about 98%, about 99% or 100% homologous (e.g., identical) to the sequence having a NCBI Reference No: P43489 or NP_003318 (SEQ ID NO: 45) or a fragment thereof, and/or may optionally comprise up to one or up to two or up to three conservative amino acid substitutions.

SEQ ID NO: 45 is provided below:

[SEQ ID NO: 45]

| | |
|---|---|
| 1 | MCVGARRLGR GPCAALLLLG LGLSTVTGLH |
| | CVGDTYPSND RCCHECRPGN GMVSRCSRSQ |
| 61 | NTVCRPCGPG FYNDVVSSKP CKPCTWCNLR |
| | SGSERKQLCT ATQDTVCRCR AGTQPLDSYK |
| 121 | PGVDCAPCPP GHFSPGDNQA CKPWTNCTLA |
| | GKHTLQPASN SSDAICEDRD PPATQPQETQ |
| 181 | GPPARPITVQ PTEAWPRTSQ GPSTRPVEVP |
| | GGRAVAAILG LGLVLGLLGP LAILLALYLL |
| 241 | RRDQRLPPDA HKPPGGGSFR TPIQEEQADA |
| | HSTLAKI |

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous (e.g., identical) to the sequence having a NCBI Reference No: NP_036224 (SEQ ID NO: 46) or a fragment thereof, and/or may optionally comprise up to one or up to two or up to three conservative amino acid substitutions.

SEQ ID NO: 46 is provided below:

[SEQ ID NO: 46]

| | |
|---|---|
| 1 | MKSGLWYFFL FCLRIKVLTG EINGSANYEM |
| | FIFHNGGVQI LCKYPDIVQQ FKMQLLKGGQ |
| 61 | ILCDLIKTKG SGNTVSIKSL KFCHSQLSNN |
| | SVSFFLYNLD HSHANYYFCN LSIFDPPPFK |
| 121 | VTLIGGYLHI YESQLCCQLK FWLPIGCAAF |
| | VVVCILGCIL ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

In certain embodiments, the intracellular signaling domain of the CAR comprises a co-stimulatory signaling region that comprises a CD28 polypeptide. In certain embodiments, the intracellular signaling domain of the CAR comprises an intracellular domain of a human CD28 or a portion thereof. The CD28 polypeptide can comprise or have an amino acid sequence that is at least about 85%, about 90%, about 95%, about 96%, about 97%, about 98%, about 99% or 100% homologous (e.g., identical) to the amino acid sequence set forth in SEQ ID NO: 15, or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 15 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, or 200 to 220 of SEQ ID NO: 15. In certain embodiments, the intracellular signaling domain of the CAR comprises a co-stimulatory signaling region that comprises a CD28 polypeptide comprising or having an amino acid sequence of amino acids 180 to 220 of SEQ ID NO: 15.

In certain embodiments, the intracellular signaling domain of the CAR comprises an intracellular domain of a mouse CD28 or a portion thereof. In certain embodiments, the CD28 polypeptide comprises or has an amino acid sequence that is at least about 85%, about 90%, about 95%, about 96%, about 97%, about 98%, about 99% or about 100% homologous to the sequence having a NCBI Reference No: NP_031668.3 (SEQ ID NO: 47) or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 47 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 114 to 220, 150 to 200, 178 to 218, or 200 to 220 of SEQ ID NO: 47. In certain embodiments, the co-stimulatory signaling region of a presently disclosed CAR comprises a CD28 polypeptide that comprises or has the amino acids 178 to 218 of SEQ ID NO: 47.

SEQ ID NO: 47 is provided below:

[SEQ ID NO: 47]

| | |
|---|---|
| 1 | MTLRLLFLAL NFFSVQVTEN KILVKQSPLL |
| | VVDSNEVSLS CRYSYNLLAK EFRASLYKGV |
| 61 | NSDVEVCVGN GNFTYQPQFR SNAEFNCDGD |
| | FDNETVTFRL WNLHVNHTDI YFCKIEFMYP |
| 121 | PPYLDNERSN GTIIHIKEKH LCHTQSSPKL |
| | FWALVVVAGV LFCYGLLVTV ALCVIWTNSR |
| 181 | RNRLLQSDYM NMTPRRPGLT RKPYQPYAPA |
| | RDFAAYRP |

In accordance with the presently disclosed subject matter, a “CD28 nucleic acid molecule” refers to a polynucleotide encoding a CD28 polypeptide. An exemplary nucleotide sequence that encodes amino acids 178 to 218 of SEQ ID NO: 47) is set forth in SEQ ID NO: 48, which is provided below.

[SEQ ID NO: 48]
aat agtagaagga acagactcct tcaaagtgac
tacatgaaca tgactccccg gaggcctggg
ctcactcgaa agccttacca gccctacgcc
cctgccagag actttgcagc gtaccgcccc

In certain embodiments, the intracellular signaling domain of the CAR comprises an intracellular domain of a murine CD28 or a portion thereof. The intracellular domain of murine CD28 can comprise or have an amino acid sequence that is at least about 85%, about 90%, about 95%, about 96%, about 97%, about 98%, about 99% or about 100% homologous (e.g., identical) to SEQ ID NO: 49 or a fragment thereof, and/or may optionally comprise up to one or up to two or up to three conservative amino acid substitutions. SEQ ID NO: 49 is provided below:

[SEQ ID NO: 49]
NSRRNRLLQS DYMNMTPRRP GLTRKPYQPY APARDFAAYR P.

An exemplary nucleic acid sequence encoding the amino acid sequence of SEQ ID NO: 49 is set forth in SEQ ID NO: 50, which is provided below.

[SEQ ID NO: 50]
AATAGTAGAAGGAACAGACTCCTTCAAAGTGACTACA
TGAACATGACTCCCCGGAGGCCTGGGCTCACTCGAAA
GCCTTACCAGCCCTACGCCCCTGCCAGAGACTTTGCA
GCGTACCGCCCC

In certain embodiments, the intracellular signaling domain of the CAR comprises an intracellular domain of human CD28 or a portion thereof. The intracellular domain of human CD28 can comprise or have an amino acid sequence that is at least about 85%, about 90%, about 95%, about 96%, about 97%, about 98%, about 99% or about 100% homologous (e.g., identical) to SEQ ID NO: 51 or fragments thereof, and/or may optionally comprise up to one or up to two or up to three conservative amino acid substitutions. SEQ ID NO: 51 is provided below:

[SEQ ID NO: 51]
RSKRSRLLHS DYMNMTPRRP GPTRKHYQPY APPRDFAAYR S.

An exemplary nucleic acid sequence encoding the amino acid sequence of SEQ ID NO: 51 is set forth in SEQ ID NO: 52, which is provided below.

[SEQ ID NO: 52]
AGGAGTAAGAGGAGCAGGCTCCTGCACAGTGACTACA
TGAACATGACTCCCCGCCGCCCCGGGCCCACCCGCAA
GCATTACCAGCCCTATGCCCCACCACGCGACTTCGCA
GCCTATCGCTCC

In certain embodiments, mutation sites and/or junction between domains/motifs/regions of the CAR derived from different proteins are de-immunized. Immunogenicity of junctions between different CAR moieties can be predicted using NetMHC 4.0 Server. For each peptide containing at least one amino acid from next moiety, binding affinity to HLA A, B and C, for all alleles, can be predicted. A score of immunogenicity of each peptide can be assigned for each peptide. Immunogenicity score can be calculated using the formula Immunogenicity score=[(50-binding affinity)*HLA frequency]ₙ. n is the number of prediction for each peptide.

In certain embodiments, the CAR comprises an extracellular antigen-binding region that comprises a human scFv that specifically binds to human Sialyl Lewis A, a transmembrane domain comprising a CD28 polypeptide, a CD8 polypeptide, or a CD166 polypeptide, and an intracellular domain comprising a wild-type or modified CD3ζ polypeptide and a co-stimulatory signaling region that comprises a CD28 polypeptide or a 4-1BB polypeptide. The CAR also comprises a signal peptide or a leader covalently joined to the 5′ terminus of the extracellular antigen-binding domain. The signal peptide comprises amino acids having the sequence set forth in SEQ ID NO: 13. In certain embodiments, the human scFv is scFv 5B1, whose variable region sequences are provided in Table 1.

In some embodiments, the CAR of the presently disclosed subject matter further comprises an inducible promoter, for expressing nucleic acid sequences in human cells. Promoters for use in expressing CAR genes can be a constitutive promoter, such as ubiquitin C (UbiC) promoter.

The presently disclosed subject matter also provides nucleic acid molecules encoding the Sialyl Lewis A-targeted CAR described herein or a functional portion thereof. In certain embodiments, the nucleic acid molecule encodes a presently disclosed Sialyl Lewis A-targeted CAR comprising a human scFv that specifically binds to human Sialyl Lewis A, a transmembrane domain comprising a CD28 polypeptide, a CD8 polypeptide, or a CD166 polypeptide, and an intracellular domain comprising a wild-type or modified CD3ζ polypeptide and a co-stimulatory signaling region comprising a CD28 polypeptide or a 4-1BB polypeptide.

In certain embodiments, the nucleic acid molecule encodes a Sialyl Lewis A-targeted CAR comprising a human scFv that comprises a heavy chain variable region comprising the amino acid sequence set forth in SEQ ID NO: 7, a light chain variable region comprising the amino acid sequence set forth in SEQ ID NO: 8, and a linker having the amino acid sequence set forth in SEQ ID NO: 11 positioned between the heavy chain variable region and the light chain variable region, a transmembrane domain comprising a CD28 polypeptide, a CD8 polypeptide, or a CD166 polypeptide, and an intracellular domain comprising a wild-type or modified CD3ζ polypeptide and a co-stimulatory signaling region comprising a CD28 polypeptide or a 4-1BB polypeptide.

In certain embodiments, the nucleic acid molecule encodes a functional portion of a presently disclosed Sialyl Lewis A-targeted CAR. As used herein, the term “functional portion” refers to any portion, part or fragment of a presently disclosed Sialyl Lewis A-targeted CAR, which portion, part or fragment retains the biological activity of the Sialyl Lewis A-targeted CAR (the parent CAR). For example, functional portions encompass the portions, parts or fragments of a presently disclosed Sialyl Lewis A-targeted CAR that retains the ability to recognize a target cell, to treat a disease, to a similar, same, or even a higher extent as the parent CAR. In certain embodiments, an isolated nucleic acid molecule encoding a functional portion of a presently disclosed Sialyl Lewis A-targeted CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

### V. Immunoresponsive Cells

The presently disclosed subject matter provides cells comprising a presently disclosed Sialyl Lewis A-targeted CAR, and methods of using such cells for treating malignant growth, e.g., for treating cancer such as pancreatic cancer. For example, in some embodiments, provided herein is a T cell comprising a chimeric antigen receptor that recognizes Sialyl Lewis A disclosed herein. Such cells are administered to a human subject in need thereof for treating and/or preventing malignant growth of a tumor such as a solid tumor, e.g., pancreatic cancer.

In some embodiments, CARs described herein can be delivered to immunoresponsive cells by appropriate means known to a skilled artisan. For example, in some embodiments, a CAR described herein can be delivered to an immunoresponsive cell by a vector or other delivery vehicle. In some embodiments, a CAR described herein can be delivered to an immunoresponsive cell in the form of an RNA (e.g., mRNA) construct. In some embodiments, an immunoresponsive cell can be transduced with a presently disclosed CAR by way of a viral vector (e.g., a retroviral vector) such that the cells express the CAR. The presently disclosed subject matter also provides methods of using such cells for the treatment of a tumor or a solid tumor, e.g., pancreatic cancer.

The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of any polypeptide or system disclosed herein. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the CAR-expressing T cells express Foxp3 to achieve and maintain a T regulatory phenotype.

In certain embodiments, the cell is a Natural killer cell. Natural killer (NK) cells can be lymphocytes that are part of cell-mediated immunity and act during the innate immune response. NK cells do not require prior activation in order to perform their cytotoxic effect on target cells.

The immunoresponsive cells of the presently disclosed subject matter can express an extracellular antigen-binding domain (e.g., a human scFV, a Fab that is optionally crosslinked, or a F(ab)₂) that specifically binds to Sialyl Lewis A (e.g., human Sialyl Lewis A), for the treatment of cancer, e.g., pancreatic cancer. Such immunoresponsive cells can be administered to a subject (e.g., a human subject) in need thereof for the treatment of cancer. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

A presently disclosed immunoresponsive cell can further include at least one recombinant or exogenous co-stimulatory ligand. For example, a presently disclosed immunoresponsive cell can be further transduced with at least one co-stimulatory ligand, such that the immunoresponsive cell co-expresses or is induced to co-express the Sialyl Lewis A-targeted CAR and the at least one co-stimulatory ligand. The interaction between the Sialyl Lewis A-targeted CAR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNFβ)/lymphotoxin-alpha (LTα), lymphotoxin-beta (LT(3), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the immunoresponsive cell comprises one recombinant co-stimulatory ligand that is 4-1BBL. In certain embodiments, the immunoresponsive cell comprises two recombinant co-stimulatory ligands that are 4-1BBL and CD80. Immunoresponsive cells comprising a CAR and at least one recombinant co-stimulatory ligand are described in U.S. Pat. No. 8,389,282 and U.S. Patent Publication No. 2016/0045551, both of which are incorporated by reference in their entirety. In certain embodiments, the immunoresponsive cell comprises a presently disclosed Sialyl Lewis A-targeted CAR and a recombinant cytokine (e.g., IL-12). In certain embodiments, the immunoresponsive cell comprises a presently disclosed Sialyl Lewis A-targeted CAR and a recombinant CD40L polypeptide.

Furthermore, a presently disclosed immunoresponsive cell can further comprise at least one exogenous cytokine. For example, a presently disclosed immunoresponsive cell can be further transduced with at least one cytokine, such that the immunoresponsive cell secretes the at least one cytokine as well as expresses the Sialyl Lewis A-targeted CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The Sialyl Lewis A-specific or Sialyl Lewis A-targeted human lymphocytes that can be used in peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, a presently disclosed immunoresponsive cell (e.g., T cell) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers/cell of a presently disclosed Sialyl Lewis A-targeted CAR.

Additionally, the immunoresponsive cells can comprise and express (e.g., naturally or modified to express) an antigen recognizing receptor that binds to a second antigen that is different than Sialyl Lewis A (e.g., human Sialyl Lewis A). The inclusion of an antigen recognizing receptor in addition to a presently disclosed CAR on the immunoresponsive cell can increase the avidity of the CAR or the immunoresponsive cell comprising thereof on a targeted cell, especially, the CAR is one that has a low binding affinity to Sialyl Lewis A (e.g., human Sialyl Lewis A), e.g., a K_{d} of about 2×10⁻⁸M or more, about 5×10⁻⁸M or more, about 8×10⁻⁸M or more, about 9×10⁻⁸M or more, about 1×10⁻⁷ M or more, about 2×10⁻⁷M or more, or about 5×10⁻⁷M or more.

In certain embodiments, the antigen recognizing receptor is a chimeric co-stimulatory receptor (CCR). As used herein, the term “chimeric co-stimulatory receptor” or “CCR” refers to a chimeric receptor that binds to an antigen and provides co-stimulatory signals, but does not provide a T-cell activation signal. CCR is described in Krause, et al., J. Exp. Med. (1998); 188(4):619-626, and US20020018783, the contents of which are incorporated by reference in their entireties. CCRs mimic co-stimulatory signals, but unlike, CARs, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen (e.g., Sialyl Lewis A), whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises an extracellular antigen-binding domain that binds to an antigen different than Sialyl Lewis A, a transmembrane domain, and a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second antigen is selected so that expression of both Sialyl Lewis A and the second antigen is restricted to the targeted cells (e.g., cancerous tissue or cancerous cells). Similar to a CAR, the extracellular antigen-binding domain can be a scFv, a Fab, a F(ab)₂, or a fusion protein with a heterologous sequence to form the extracellular antigen-binding domain. In certain embodiments, the CCR binds to a pancreatic cancer-specific antigen.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells (e.g., pancreatic cancer cells), e.g., a pancreatic cancer-specific antigen. The truncated CAR functions as an adhesion molecule that enhances the avidity of a presently disclosed CAR, especially, one that has a low binding affinity to Sialyl Lewis A, thereby improving the efficacy of the presently disclosed CAR or immunoresponsive cell (e.g., T cell) comprising thereof. In certain embodiments, a presently disclosed T cell comprises or is transduced to express a presently disclosed CAR targeting Sialyl Lewis A and a truncated CAR targeting a pancreatic cancer-specific antigen.

### VI. Nucleic Acid Compositions and Vectors

The presently disclosed subject matter provides nucleic acid compositions comprising a polynucleotide encoding the CAR disclosed herein. Also provided are cells comprising such nucleic acid compositions.

Genetic modification of immunoresponsive cells (e.g., T cells, NK cells) can be accomplished by delivering a recombinant DNA or RNA construct encoding a CAR into the target cells of a substantially homogeneous cell composition. In some embodiments, such a recombinant DNA or RNA construct can be delivered into immunoresponsive cells using a vector. In some embodiments, such a vector can be a retroviral vector (e.g., gamma retroviral), which is employed for the introduction of the DNA or RNA construct into the host cell genome. For example, a polynucleotide encoding the Sialyl Lewis A-targeted CAR can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide Sialyl Lewis A-targeted CAR expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Transducing viral vectors can be used to express a co-stimulatory ligand and/or secrets a cytokine (e.g., 4-1BBL and/or IL-12) in an immunoresponsive cell. Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263 267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

In certain non-limiting embodiments, the vector expressing a presently disclosed Sialyl Lewis A-targeted CAR is a retroviral vector, e.g., an oncoretroviral vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1α enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above. The resulting cells can be grown under conditions similar to those for unmodified cells, whereby the modified cells can be expanded and used for a variety of purposes.

### VII. Genomic Integration into Immunoresponsive Cell

In certain embodiments, a presently disclosed Sialyl Lewis A-targeted CAR can be integrated into a selected locus of the genome of an immunoresponsive cell. Any targeted genome editing methods can be used to integrate the CAR in selected loci of the genome of an immunoresponsive cell. In certain embodiments, the expression of the presently disclosed Sialyl Lewis A-targeted CAR is driven by an endogenous promoter/enhancer within or near the locus. In certain embodiments, the expression of the presently disclosed Sialyl Lewis A-targeted CAR is driven by an exogenous promoter integrated into the locus. The locus where the presently disclosed Sialyl Lewis A-targeted CAR is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In certain embodiments, the CRISPR system is used to integrate the presently disclosed Sialyl Lewis A-targeted CAR in selected loci of the genome of an immunoresponsive cell. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. Methods of using the CRISPR system are described, for example, in WO 2014093661 A2, WO 2015123339 A1 and WO 2015089354 A1, which are incorporated by reference in their entireties.

In certain embodiments, zinc-finger nucleases are used to integrate the presently disclosed Sialyl Lewis A-targeted CAR in selected loci of the genome of an immunoresponsive cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Methods of using the ZFN system are described, for example, in WO 2009146179 A1, WO 2008060510 A2 and CN 102174576 A, which are incorporated by reference in their entireties.

In certain embodiments, the TALEN system is used to integrate the presently disclosed Sialyl Lewis A-targeted CAR in selected loci of the genome of an immunoresponsive cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. Methods of using the TALEN system are described, for example, in WO 2014134412 A1, WO 2013163628 A2 and WO 2014040370 A1, which are incorporated by reference in their entireties. Methods for delivering the genome editing agents can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within the selected locus, or anywhere that can influence gene expression of the integrated Sialyl Lewis A-targeted CAR. In certain embodiments, the modification is introduced upstream of the transcriptional start site of the integrated Sialyl Lewis A-targeted CAR. In certain embodiments, the modification is introduced between the transcriptional start site and the protein coding region of the integrated Sialyl Lewis A-targeted CAR. In certain embodiments, the modification is introduced downstream of the protein coding region of the integrated presently disclosed Sialyl Lewis A-targeted CAR.

### VIII. Polypeptides and Analogs and Polynucleotides

Also included in the presently disclosed subject matter are extracellular antigen-binding domains that specifically binds to an Sialyl Lewis A (e.g., an scFv (e.g., a human scFv), a Fab, or a (Fab)₂), CD3ζ, CD8, CD28, etc. polypeptides or fragments thereof, and polynucleotides encoding thereof that are modified in ways that enhance their anti-tumor activity when expressed in an immunoresponsive cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding domain that specifically binds to Sialyl Lewis A (e.g., human Sialyl Lewis A) (e.g., an scFv (e.g., a human scFv), a Fab, or a (Fab)₂), CD3ζ, CD8, CD28) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### IX. Administration

Sialyl Lewis A-targeted CARs and immunoresponsive cells comprising thereof of the presently disclosed subject matter can be provided systemically or directly to a subject for treating or preventing a neoplasia. In certain embodiments, Sialyl Lewis A-targeted CARs, and immunoresponsive cells comprising thereof are directly injected into an organ of interest (e.g., an organ affected by a neoplasia). Alternatively or additionally, Sialyl Lewis A-targeted CARs and immunoresponsive cells comprising thereof are provided indirectly to the organ of interest, for example, by administration into the circulatory system (e.g., the tumor vasculature). Expansion and differentiation agents can be provided prior to, during or after administration of cells and compositions to increase production of T cells in vitro or in vivo.

Sialyl Lewis A-targeted CARs and immunoresponsive cells comprising thereof of the presently disclosed subject matter can be administered in any physiologically acceptable vehicle, normally intravascularly, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising immunoresponsive cells comprising a presently disclosed Sialyl Lewis A-targeted CAR can comprise a purified population of cells. Those skilled in the art can readily determine the percentage of immunoresponsive cells in a cell population using various well-known methods, such as fluorescence activated cell sorting (FACS). The ranges of purity in cell populations comprising immunoresponsive cells comprising a presently disclosed anti-Sialyl Lewis A-specific CAR can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The immunoresponsive cells can be introduced by injection, catheter, or the like. If desired, factors can also be included, including, but not limited to, interleukins, e.g. IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising immunoresponsive cells comprising a presently disclosed Sialyl Lewis A-targeted CAR and a pharmaceutically acceptable carrier. Administration can be autologous or non-autologous. For example, immunoresponsive cells comprising a presently disclosed Sialyl Lewis A-targeted CAR and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising immunoresponsive cells comprising a presently disclosed Sialyl Lewis A-targeted CAR), it can be formulated in a unit dosage injectable form (solution, suspension, emulsion).

In certain embodiments, compositions of the presently disclosed subject matter can comprise one or more antigen-binding proteins such as an anti-Sialyl Lewis A antibody or an antigen-binding fragment thereof, disclosed herein, and a pharmaceutically acceptable carrier.

### XI. Formulations

Immunoresponsive cells comprising a presently disclosed Sialyl Lewis A-targeted CAR and compositions comprising thereof of the presently disclosed subject matter can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising immunoresponsive cells expressing a presently disclosed Sialyl Lewis A-targeted CAR, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the immunoresponsive cells expressing a generally Sialyl Lewis A-targeted CAR of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the immunoresponsive cells as describe in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the immunoresponsive cells of the presently disclosed subject matter is the quantity of cells necessary to achieve an optimal effect. The quantity of cells to be administered will vary for the subject being treated. In certain embodiments, from about 10⁴ to about 10¹⁰, from about 10⁵ to about 10⁹, or from about 10⁶ to about 10⁸ immunoresponsive cells of the presently disclosed subject matter are administered to a subject. More effective cells may be administered in even smaller numbers. In some embodiments, at least about 1×10⁸, about 2×10⁸, about 3×10⁸, about 4×10⁸, and about 5×10⁸ immunoresponsive cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### XII. Methods of Treatment

Provided herein are methods for treating a malignant growth in a subject. The methods comprise administering the presently disclosed cells comprising one or more CARs described herein in an amount effective to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

For adoptive immunotherapy using antigen-specific T cells, cell doses in the range of about 10⁶ to about 10¹⁰ (e.g., about 10⁹ or about 10⁶) are typically infused. Upon administration of the immunoresponsive cells into the subject and subsequent differentiation, the immunoresponsive cells are induced that are specifically directed against one specific antigen (e.g., Sialyl Lewis A). “Induction” of T cells can include inactivation of antigen-specific T cells such as by deletion or anergy. Inactivation is particularly useful to establish or reestablish tolerance such as in autoimmune disorders. The immunoresponsive cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the immunoresponsive cells and the compositions comprising thereof are intravenously administered to the subject in need.

The presently disclosed subject matter provides various methods of using the immunoresponsive cells (e.g., T cells) comprising a presently disclosed Sialyl Lewis A-targeted CAR. For example, the presently disclosed subject matter provides methods of reducing tumor burden in a subject. In certain non-limiting examples, the method of reducing tumor burden comprises administering an effective amount of the presently disclosed immunoresponsive cell to the subject. The presently disclosed immunoresponsive cell can reduce the number of tumor cells, reduce tumor size, and/or eradicate the tumor in the subject.

The presently disclosed subject matter also provides methods of increasing or lengthening survival of a subject having a neoplasia. In certain non-limiting example, the method of increasing or lengthening survival of a subject having a neoplasia comprises administering an effective amount of the presently disclosed immunoresponsive cell to the subject. The method can reduce or eradicate tumor burden in the subject.

The presently disclosed subject matter further provides methods for treating and/or preventing a neoplasia in a subject. In certain embodiments, the method comprises administering an effective amount of the presently disclosed immunoresponsive cells to the subject.

Cancers whose growth may be inhibited using the immunoresponsive cells of the presently disclosed subject matter comprise cancers typically responsive to immunotherapy. Non-limiting examples of neoplasia, cancers and/or tumors for treatment include pancreatic cancer.

Additionally, the presently disclosed subject matter provides methods of increasing immune-activating cytokine production in response to a cancer cell in a subject. In certain embodiments, the method comprises administering the presently disclosed immunoresponsive cell to the subject. The immune-activating cytokine can be granulocyte macrophage colony stimulating factor (GM-CSF), IFN-α, IFN-β, IFN-γ, TNF-α, IL-2, IL-3, IL-6, IL-11, IL-7, IL-12, IL-15, IL-21, interferon regulatory factor 7 (IRF7), and combinations thereof. In certain embodiments, the immunoresponsive cells including a Sialyl Lewis A-specific CAR of the presently disclosed subject matter increase the production of GM-CSF, IFN-γ, and/or TNF-α.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

A second group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia. For instance, women testing positive for a genetic mutation associated with breast cancer, but still of childbearing age, can wish to receive one or more of the antigen-binding fragments described herein in treatment prophylactically to prevent the occurrence of neoplasia until it is suitable to perform preventive surgery.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the Sialyl Lewis A-targeted CAR-expressing immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the Sialyl Lewis A-targeted CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the 3′ terminus of the intracellular domain of the Sialyl Lewis A-targeted CAR. The suicide gene can be included within the vector comprising nucleic acids encoding the presently disclosed Sialyl Lewis A-targeted CARs. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed Sialyl Lewis A-targeted CAR gives an added level of safety with the ability to eliminate the majority of CAR T cells within a very short time period. A presently disclosed immunoresponsive cell (e.g., a T cell) incorporated with a suicide gene can be pre-emptively eliminated at a given timepoint post CAR T cell infusion, or eradicated at the earliest signs of toxicity.

### XIII. Kits

The presently disclosed subject matter provides kits for the treatment or prevention of a neoplasia. In certain embodiments, the kit comprises a therapeutic or prophylactic composition comprising an effective amount of an immunoresponsive cell comprising a presently disclosed Sialyl Lewis A-targeted CAR in unit dosage form. In particular embodiments, the cells further expresses at least one co-stimulatory ligand. In some embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the immunoresponsive cell can be provided together with instructions for administering the cell to a subject having or at risk of developing a neoplasia. The instructions will generally include information about the use of the composition for the treatment and/or prevention of a neoplasia. In other embodiments, the instructions include at least one of the following: description of the therapeutic agent; dosage schedule and administration for treatment or prevention of a neoplasia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1

Introduction

Strategies that improve antigen presentation, induce epitope spreading, or perpetuate existing antitumor T cell responses hold promise for combating tumor antigen escape. For example, cancer vaccines and “immunogenic” radiation (RT) activate antigen-presenting cells (APCs) to improve tumor neoantigen display to endogenous T cells (Spiotto et al., Sci Immunol (2016); 1). However, the same neoantigens must still be expressed and presented in most, if not all, tumor cells in order to obtain a complete response. In patients who have pre-existing tumor-reactive T cells, which correlates with tumor mutational burden, immune checkpoint inhibitors can relieve T cell exhaustion and provide sustained responses. However, checkpoint inhibition cannot restore T cell responses against tumor cells that do not present the recognized antigens, just as CARs cannot direct a response against tumor cells devoid of the CAR target.

The improved tumor recognition that can occur after exposure to ionizing radiation, mediated by increased APC activation, improved T cell infiltration, and enhanced HLA or CAR target expression on the tumor (Spiotto et al., Sci Immunol (2016); 1; Weiss et al., Cancer Res (2018); 78:1031-1043), faces the same challenge of antigen escape owing to antigen loss. However, it was found that tumors that have been exposed to low dose irradiation become more sensitive to CAR T cell activity, including tumor cells that lack the CAR target. Understanding this mechanism may be particularly valuable in overcoming solid tumor antigen escape.

This alternative mechanism was characterized by which tumor susceptibility to CAR T cell-mediated elimination is enhanced by radiation conditioning, and exploit it to extend the reach of CAR T cells beyond the targeted antigen. Pancreatic cancer continues to carry a dismal prognosis with little improvement over the last decades, does not have established uniformly expressed therapeutic target antigens, and is increasing in incidence. In an orthotopic pancreatic cancer model that is partially antigen-negative, a novel means to address the challenge of clonal antigen heterogeneity by combining low-dose radiation and CAR therapy was provided.

Results

Sialyl Lewis a (Le^{A})-Specific CAR T Cells are Active Against Pancreatic Tumor Cells In Vitro

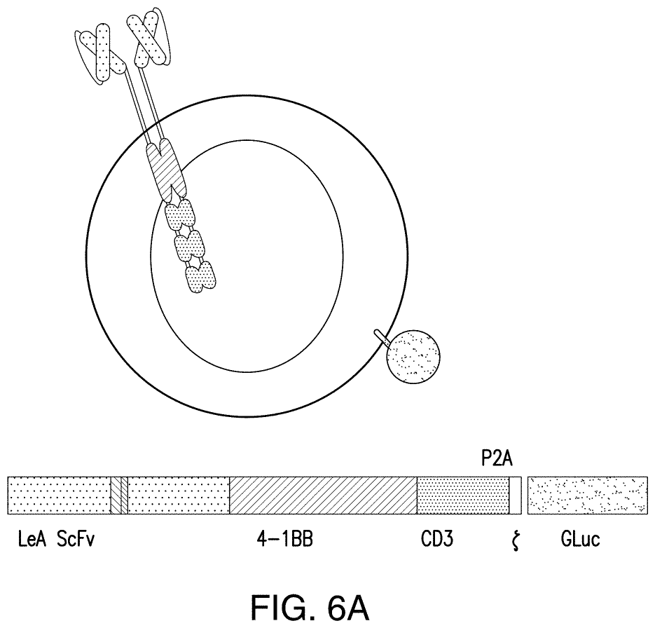

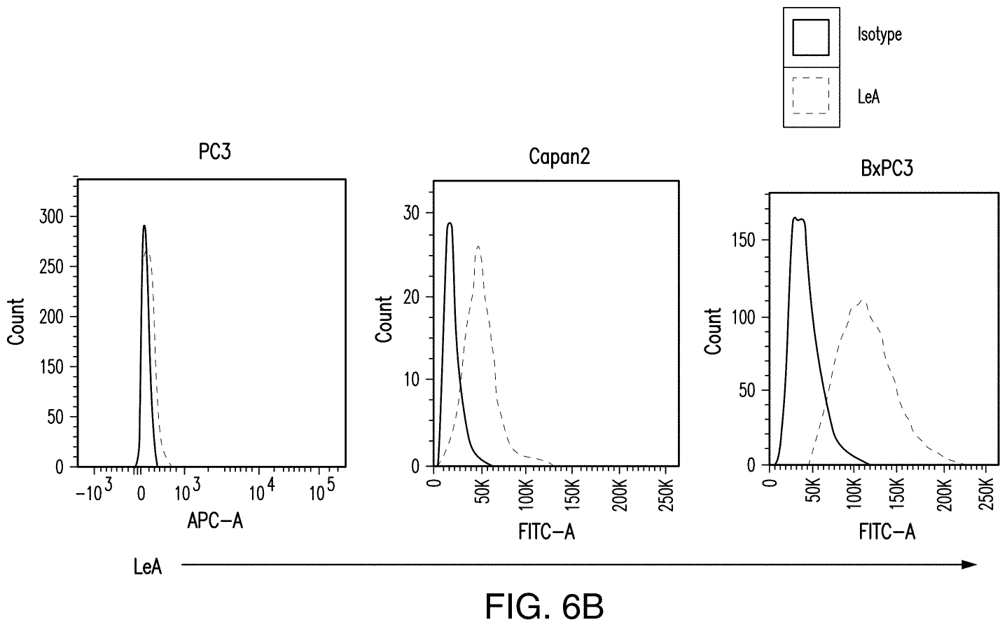

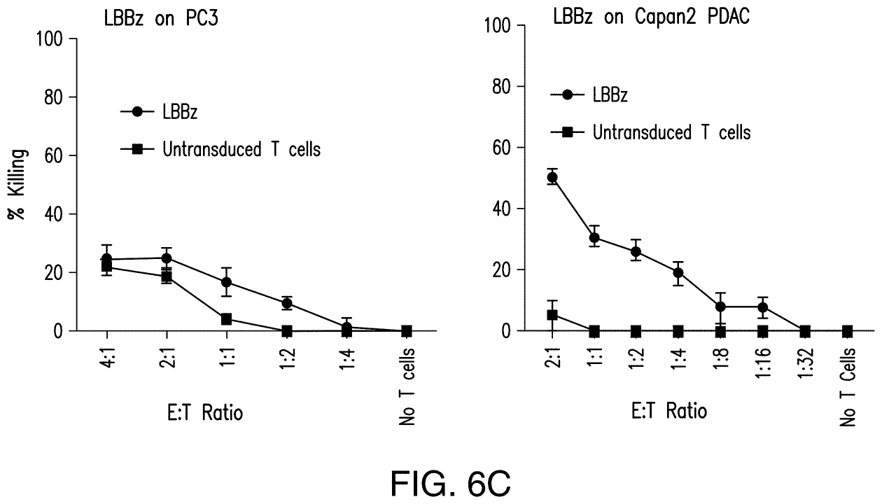

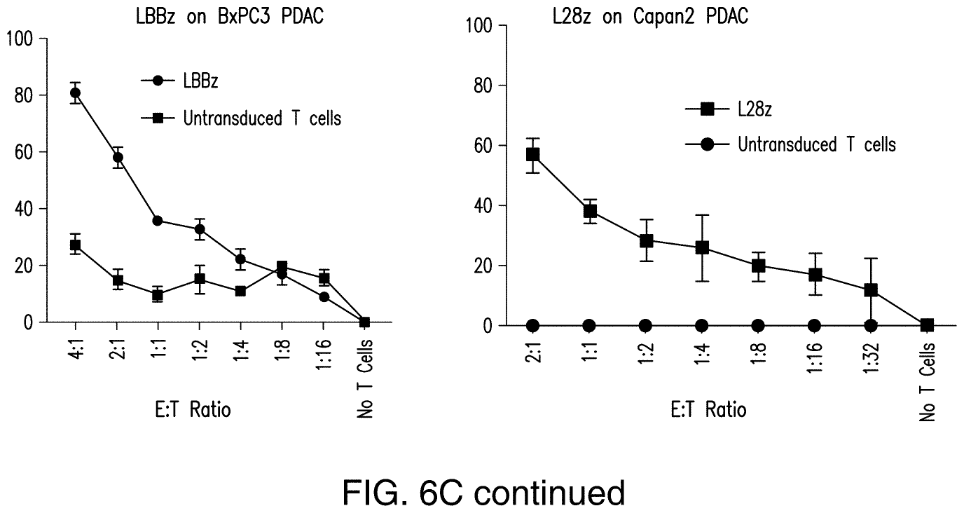

Identifying a solid tumor target that is expressed on 100% of tumor cells, and no critical normal tissues, is challenging. Pancreatic cancer exemplifies this problem, with a number of attractive targets, however none of which are clearly expressed on all tumor cells (Zhao et al., Cancer Cell (2015); 28:415-428). Sialyl Lewis A (Le^{A}), is a surface antigen expressed on 75-90% of pancreatic tumors (Viola-Villegas et al., J Nucl Med (2013); 54:1876-1882) with low expression on normal human tissues (Viola-Villegas et al., J Nucl Med (2013); 54:1876-1882), is an active antibody target in clinical trials (NCT03118349, NCT02672917, NCT02687230). The human monoclonal 5B1 antibody targeting Le^{A} has demonstrated specificity for pancreatic cancer in vitro and in vivo (Viola-Villegas et al., J Nucl Med (2013); 54:1876-1882), as well as safety and tolerability in pancreatic cancer patients at biologically active doses (O'Reilly et al., Journal of Clinical Oncology (2017); 35:4110-4110). Advanced pancreatic ductal adenocarcinoma (APDAC)-targeting CAR using this Le^{A}-specific scFv was therefore constructed. Le^{A}-specific LBBz CARs directed effective cytotoxicity against multiple pancreatic cancer tumor lines expressing Le^{A}, but not Le^{A}-negative PC3 prostate cancer cells (FIGS. 6A-6C). Capan2 PDAC expressed an intermediate level of Le^{A} (FIGS. 6A-6C), and was selected for further experiments.

Low Dose Radiation Sensitizes Tumor Cells to CAR T Cell Killing, without Inducing Target Antigen Expression

To test an initial hypothesis that radiation therapy (RT) may induce LeA expression and improve the ability of CAR T cells to eliminate tumor with heterogeneous target-antigen expression, tumor cells were irradiated with 2 Gy RT, and two days later performed a cytotoxic T lymphocyte (CTL) assay on remaining viable cells, as well as FACS analysis of surface target antigen expression. 2 Gy was chosen because higher RT doses induced small but significant tumor cell death, while 2 Gy resulted in no detectable difference in tumor viability (FIG. 1A). It was found 2 Gy (hereafter called “low dose RT”) increased the sensitivity of tumor cells to CAR T cell killing at every effector:target ratio (FIG. 1B), but surprisingly, did not increase target antigen expression (FIG. 1C).

Low Dose Radiation Affects Gene Sets Associated with Sensitivity to TRAIL-Mediated Death

To gain insight into potential mechanisms by which low dose RT sensitizes tumor cells to CAR T cell killing, RNAseq analysis was performed on the tumor cells before and after low dose RT. Although the RT itself was sublethal, gene set analysis revealed a high number of apoptotic pathways significantly affected by low dose RT (FIG. 1D). In particular, gene sets distinguishing tumor cells that are sensitive to TRAIL-mediated death from those that are not (Hamai et al., Oncogene (2006); 25:7618-7634)¹ emerged with the lowest false discovery rate (FDR <0.0000001 for each; 429 of 492 positive pathway members induced, and 114 of 128 negative pathway members downregulated) (FIG. 1D).

CAR T Cells Produce TRAIL Upon Target Antigen Encounter

TRAIL is a trimeric protein that induces death through two different receptors and a number of downstream signaling molecules that influence susceptibility; tumor cells are generally more sensitive to TRAIL-induced apoptosis than normal cells, but exist on a spectrum (Walczak et al., Nat Med (1999); 5:157-163). Gene set analysis suggested low dose RT may transcriptionally prime tumor cells to TRAIL-mediated death, which would only be relevant if the death ligand were present locally at sufficient levels. TRAIL production from LeA-specific CAR T cells were analyzed, and it was found the CAR T cells produce low levels of TRAIL at baseline, however upon target antigen encounter, significantly induce TRAIL mRNA and protein (FIG. 1E). In contrast, TRAIL is not induced after tumor recognition by T cells expressing a truncated CAR that lacks the signaling domain (Ldel), establishing the dependence of TRAIL induction on CAR signaling (FIG. 1F).

Antigen-Negative Tumor Cells Exposed to Low Dose RT are Susceptible to CAR T Cell TRAIL-Mediated Death

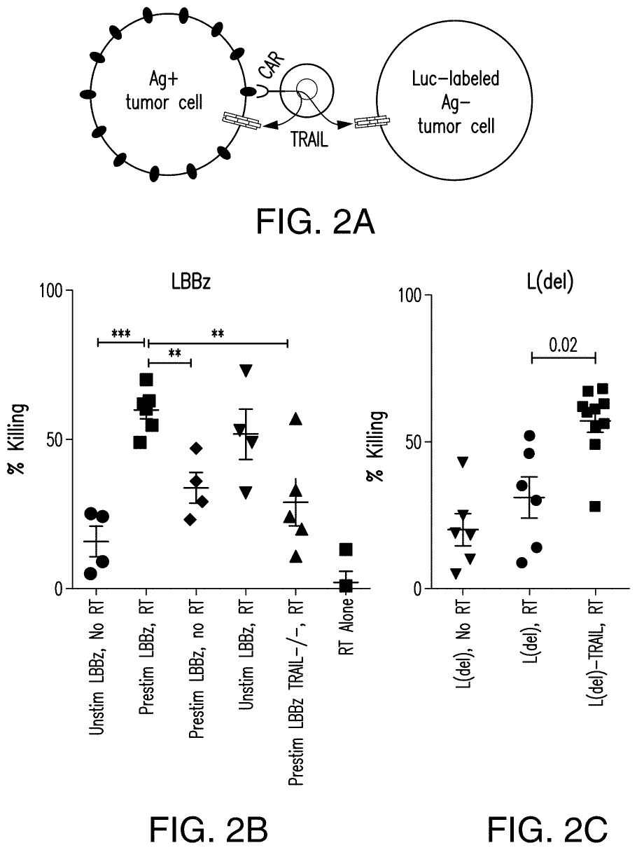

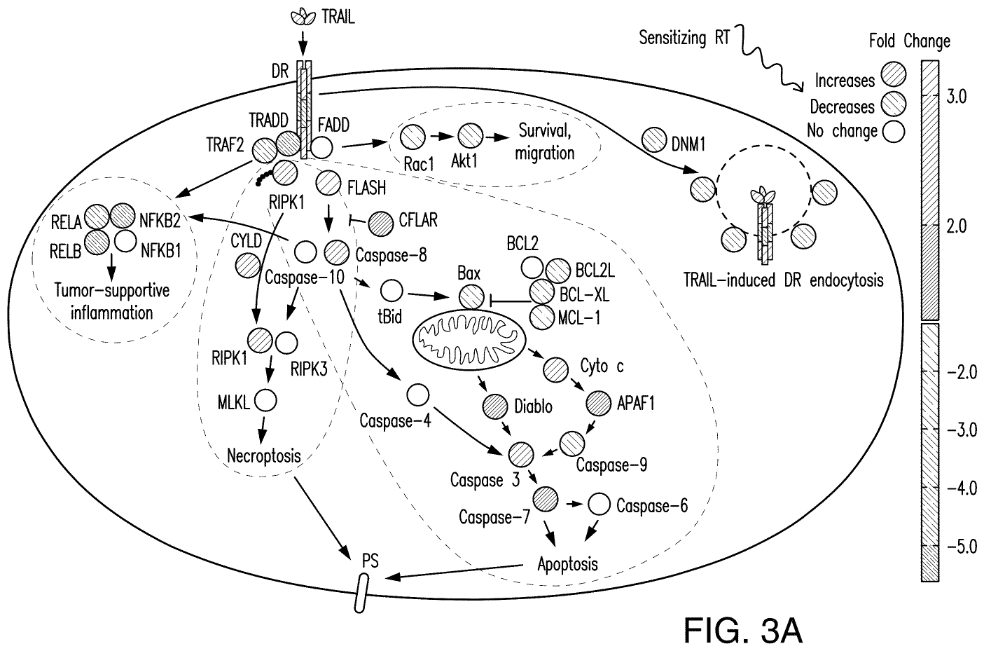

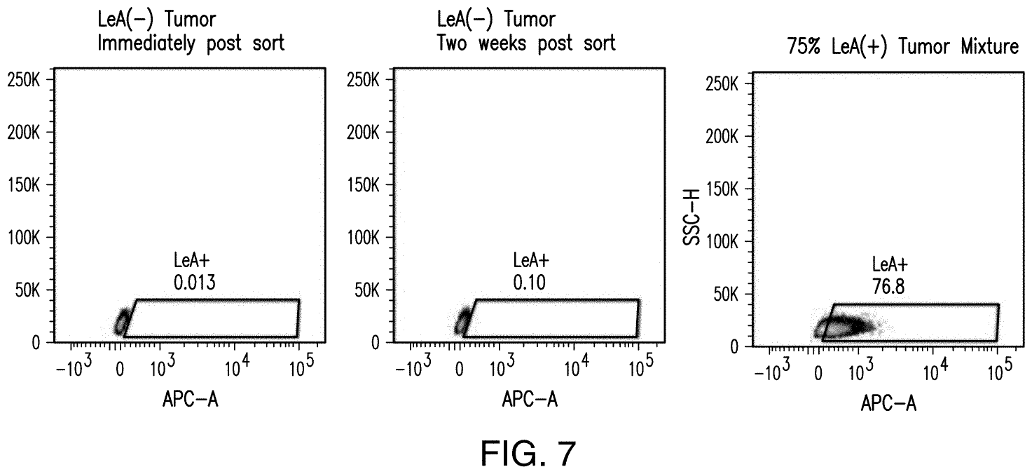

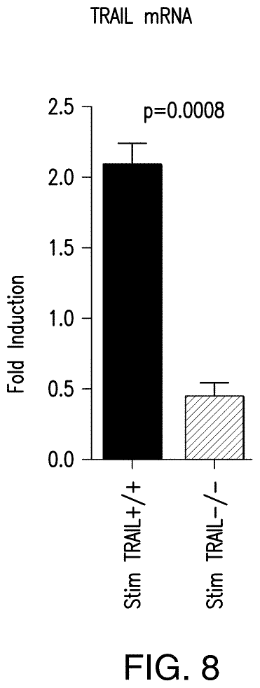

To test the functional significance of TRAIL produced by activated CAR T cells on antigen-negative tumor exposed to low dose RT, tumor cells were FACS sorted into antigen-positive (Ag⁺) and antigen-negative (Ag⁻) populations. Ag⁻ cells were transduced with Firefly Luciferase (Luc), and remained stably antigen-negative over time (FIG. 7). 75% Ag⁺ with 25% Ag⁻Luc⁺ tumor cells were mixed, exposed to low dose or no RT, and incubated with CAR T cells in which TRAIL was disrupted by CRISPR (FIGS. 2A-2B and FIG. 7). TRAIL^{wt} or knockout CAR T cells were either resting, or stimulated by their target antigen three days prior to induce TRAIL production. Using Luc activity to monitor Ag⁻ cell killing, it was found that prestimulated wt CAR T cells on RT-exposed tumor cells produced the greatest magnitude of Ag⁻ tumor cell death, which was significantly reduced by the absence of TRAIL in the CAR T cell, or the absence of sensitizing RT to the tumor (FIG. 2B). L(del) CAR T cells, which recognize the target cells but do not induce TRAIL, killed significantly more RT-sensitized Ag⁻ tumor cells if they were made to constitutively express TRAIL (FIG. 2C).

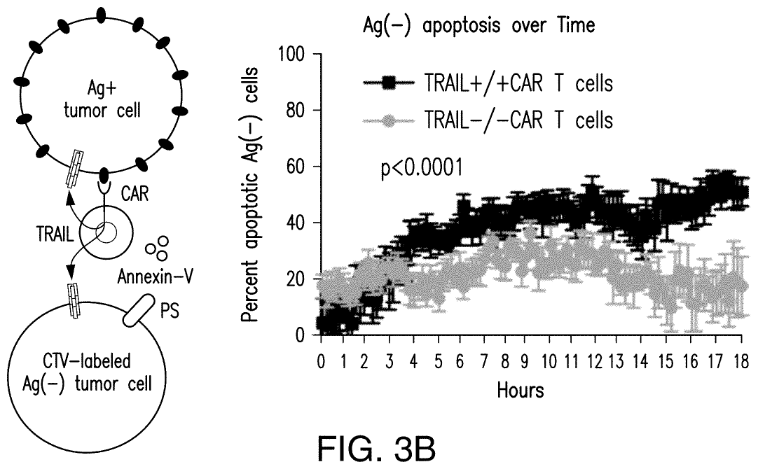

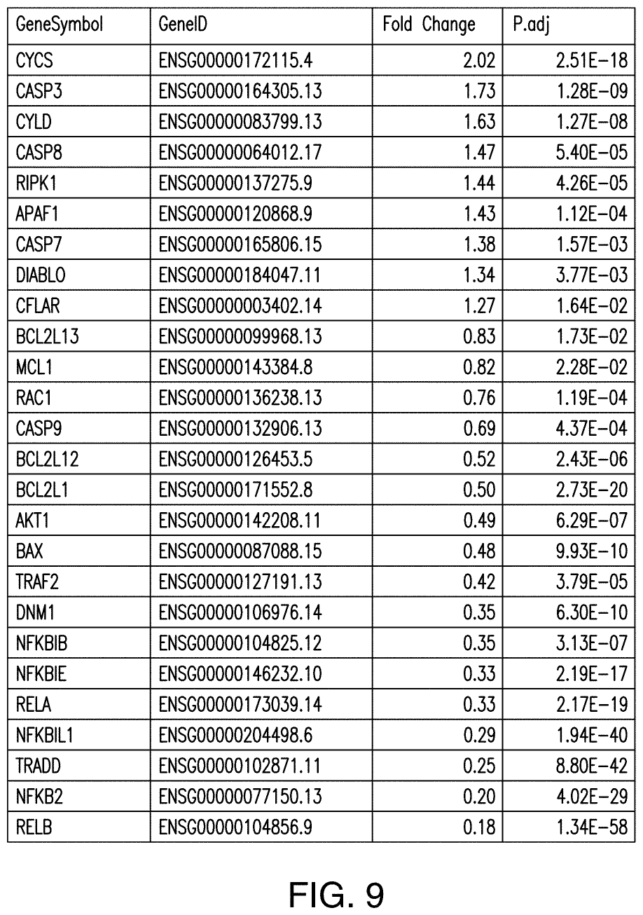

TRAIL exerts a number of context-dependent effects, including apoptosis and necroptosis of both tumor cells and T cells, or pro-tumor effects including myeloid derived suppressor cell recruitment through tumor cell NFkB activation (Hartwig et al., Mol Cell (2017); 65:730-742 e735), or survival, invasion and metastasis through Rac1 and Akt activation within the tumor (von Karstedt et al., Cancer Cell (2015); 27:561-573). To better understand how a RT-sensitized tumor might respond to increased TRAIL stimulation provided by CAR T cells, known mediators of the various downstream TRAIL signaling pathways were investigated. Many pathway mediators are regulated through transcription, cleavage, phosphorylation, ubiquitination, or other events, but gene expression analysis can provide general information regarding overall pathway activation states. Notably, gene expression changes from RNAseq data before and after sensitizing RT revealed the majority of individual members of both pro-tumor and anti-tumor mediators downstream of TRAIL were significantly altered by sensitizing RT (FIG. 3A; red or green representing significant changes, gray representing non-significant changes). Pro-survival, migration, metastasis, and tumor-supportive inflammation TRAIL pathway members were almost uniformly downregulated, while pro-apoptotic molecules were overwhelmingly induced, suggesting sensitizing RT may predispose tumor cells to TRAIL-mediated apoptosis (FIG. 3A and FIG. 9). Since apoptosis and necroptosis levels can be monitored by phosphatidylserine (PS) expression on the cell membrane, whether TRAIL produced by CAR T cells induced detectable membrane PS changes over time in Ag⁻ cells using live video microscopy of cultures containing fluorescent annexin-V antibody was tested. RT-sensitized Ag⁻ tumor cells were labeled with CellTrace Violet (CTV) before mixing with unlabeled Ag⁺ tumor cells and TRAIL^{wt} or TRAIL^{-/-} CAR T cells. Automated quantification of Ag⁻ tumor cells undergoing apoptosis demonstrated TRAIL^{-/-} CAR T cells fail to induce Ag⁻ tumor apoptosis over time, while TRAIL^{wt} CAR T cells effect steady and significant Ag⁻ tumor cell apoptosis (p<0.0001, FIG. 3B).

Pancreatic Tumor Containing a Resistant Ag⁻ Population can be Eliminated In Vivo by CAR T Cells Following Sensitizing RT

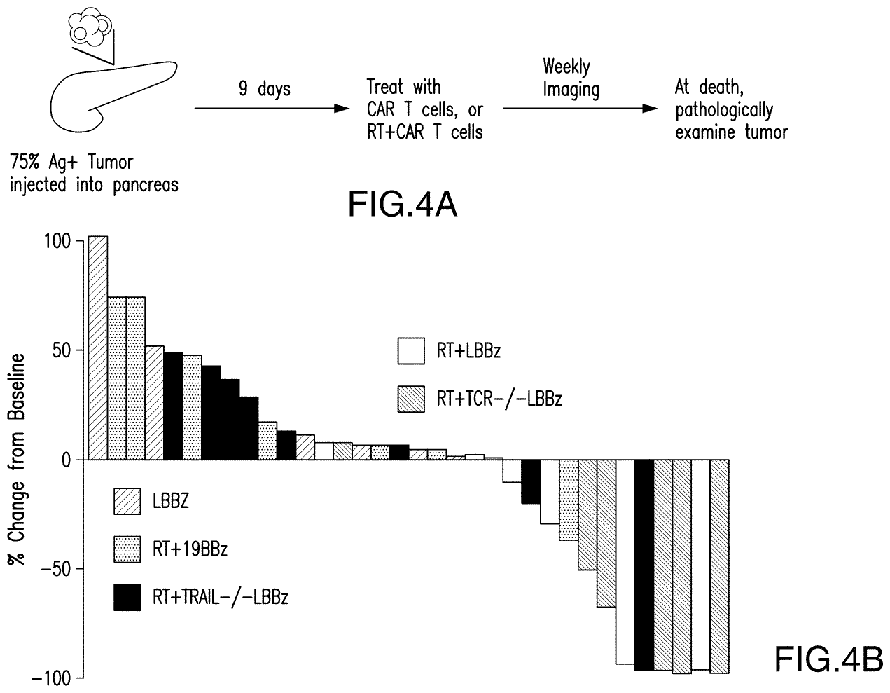

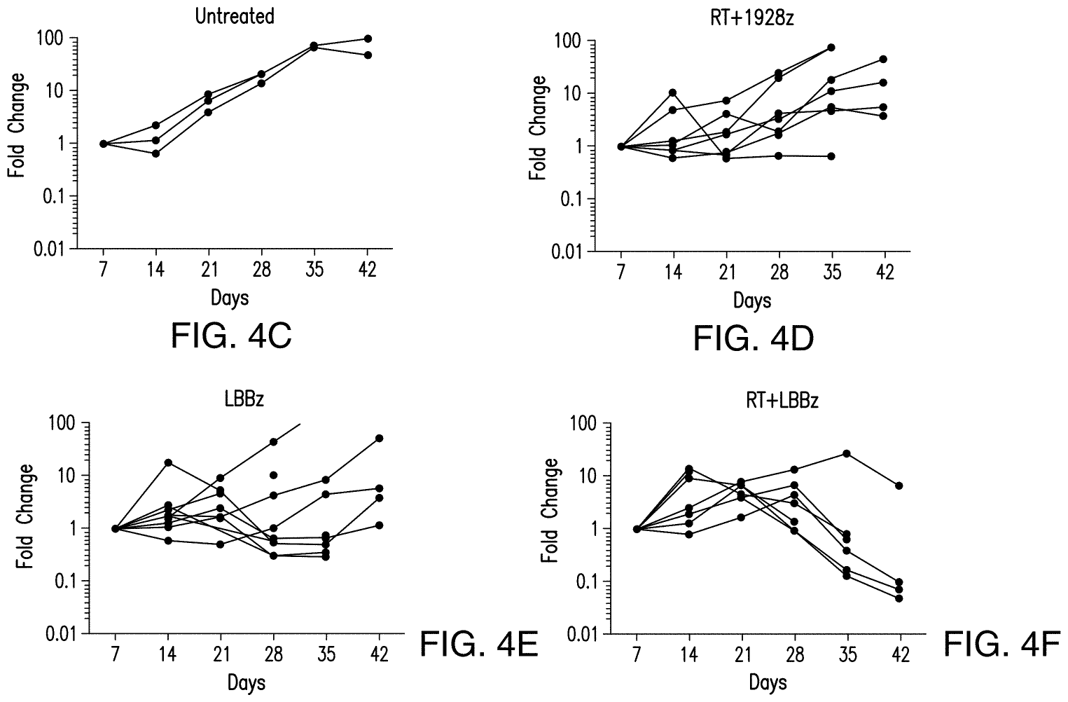

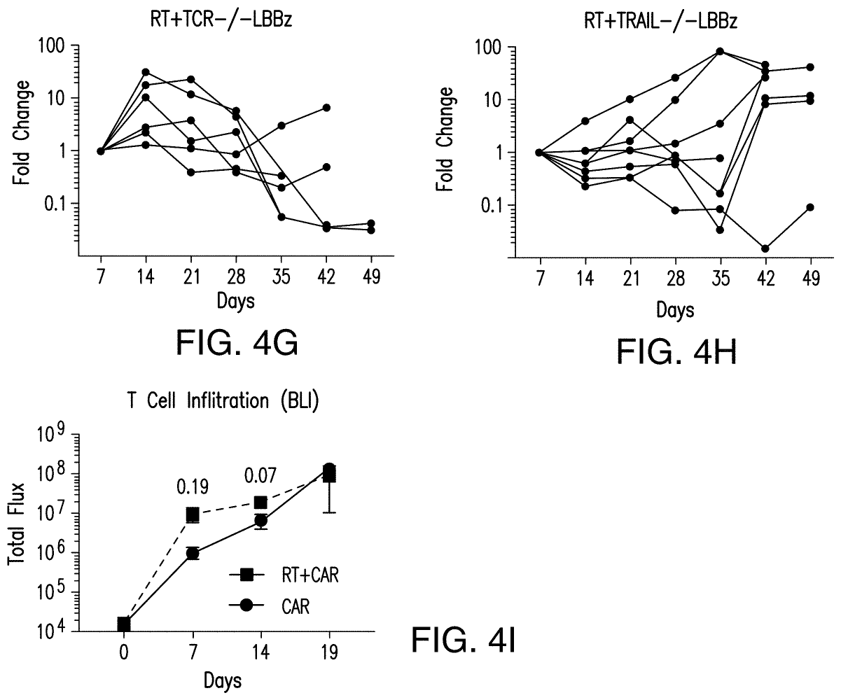

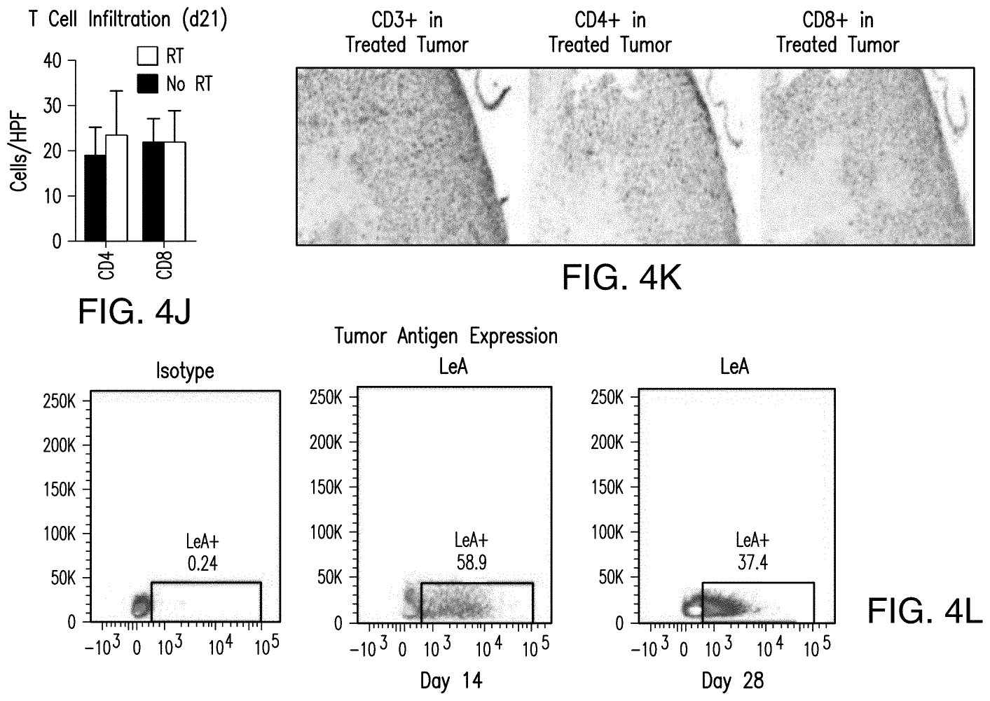

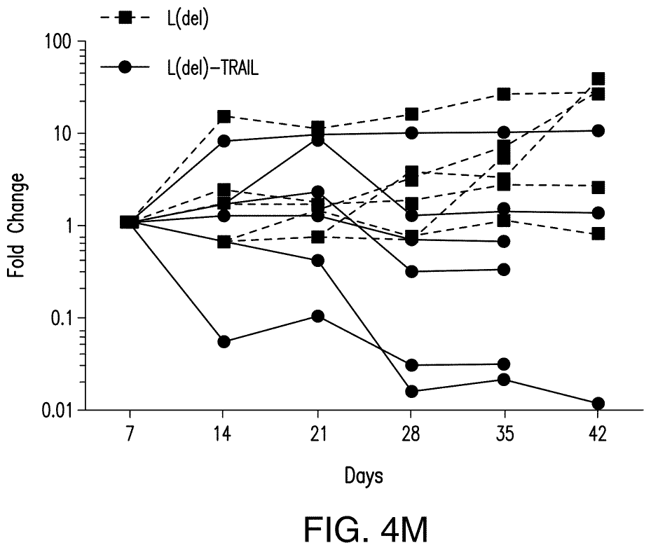

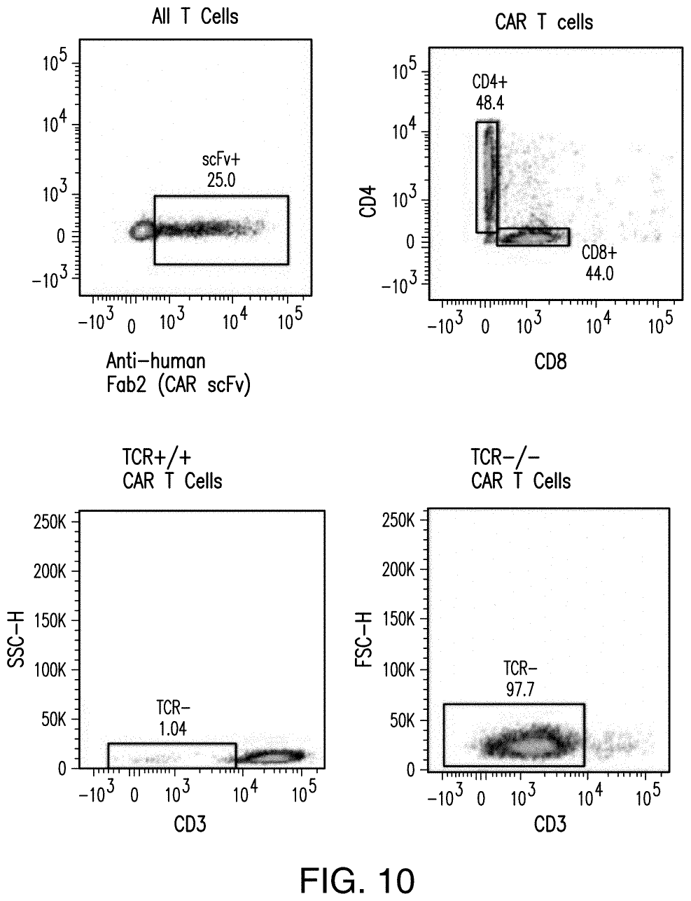

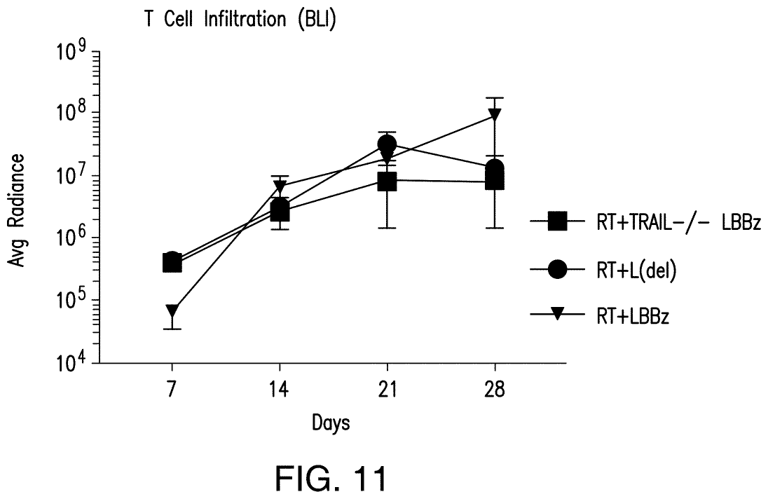

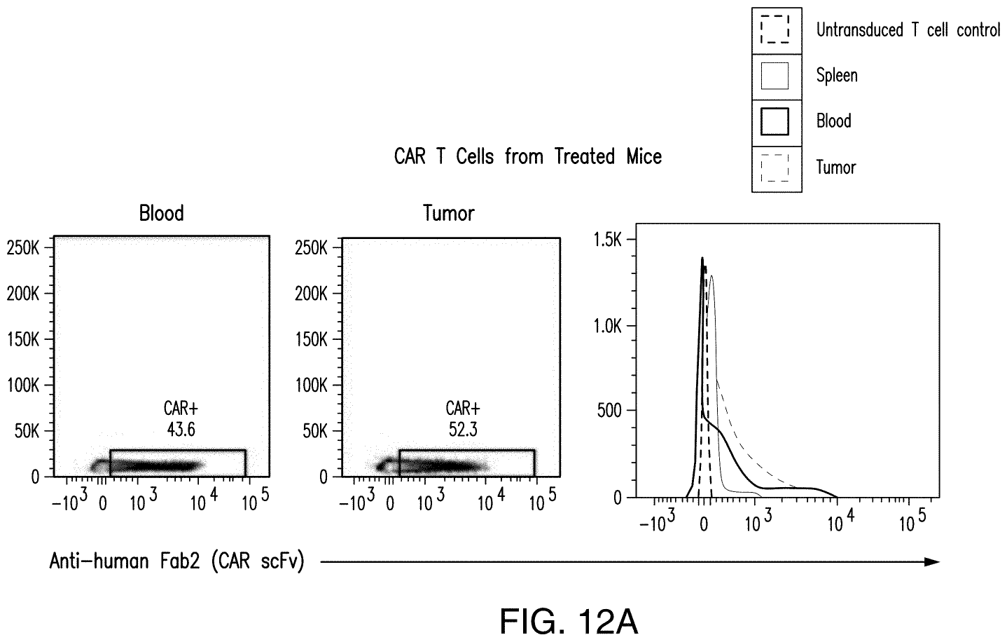

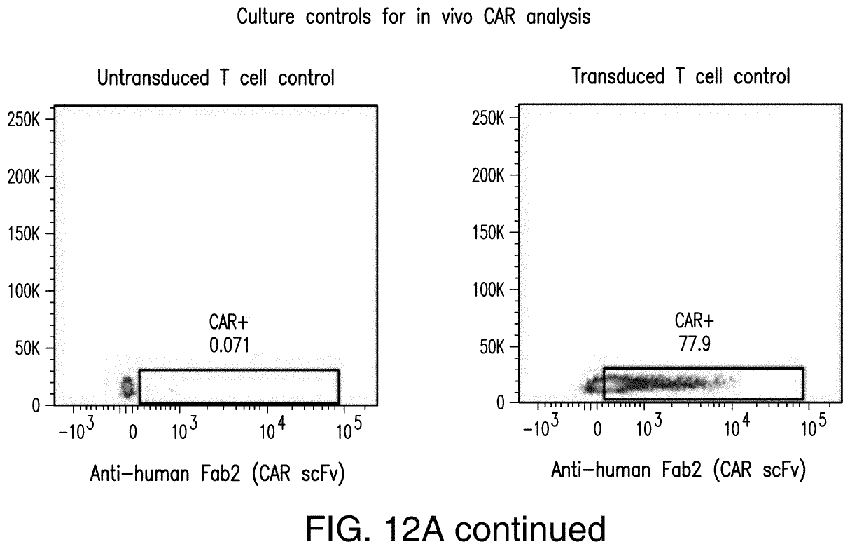

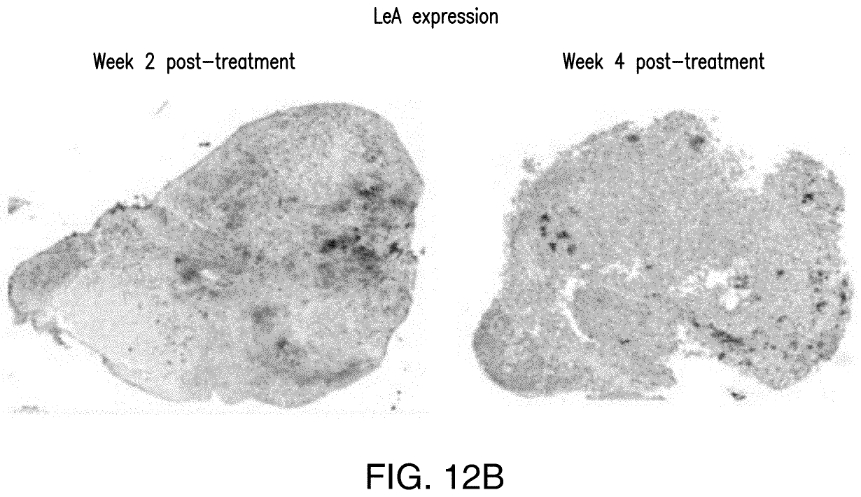

A mouse model was next established for the challenging but common clinical scenario of heterogeneous solid tumor partially devoid of target antigen. PDAC consisting of 25% Ag⁻ cells were established in the mouse pancreas, and nine days later treated with CAR T cells (FIG. 4A). CAR T cells that consistently eliminated Ag⁺ orthotopic PDAC were unable to completely eliminate any heterogeneous tumors (FIGS. 4B-4E). Whether sensitizing RT afforded any meaningful benefit to heterogeneous tumor treated with CAR T cells in vivo was tested next. Mice with established heterogeneous PDAC, treated with sensitizing RT before CAR T cells achieved more CR and PR by imaging, autopsy exam, and pathology (FIGS. 4B and 4F). Since the major known mechanism of CAR-independent T cell killing is through the T cell receptor (TCR), and RT can induce HLA expression on target cells, whether TCR-dependent tumor killing plays a significant role after sensitizing RT was tested. CAR T cells lacking their TCR (TCR^{-/-}) (FIG. 10) maintained the capacity to eliminate RT-sensitized heterogeneous tumor (FIG. 4G). RT initially resulted in moderately increased T cell accumulation within the tumor over the first two weeks (FIGS. 4I-4K). Despite significant tumor influx (FIG. 11), TRAIL^{-/-} CAR T cells failed to consistently achieve a complete response in RT-sensitized tumor-bearing mice, demonstrated by both waterfall plot of response at the time of death (which occurred from either GVHD or tumor progression) (FIG. 4B), and weekly bioluminescence imaging (FIG. 4H). Mice with tumors that relapsed/progressed still harbored CAR T cells in the blood, spleen, and tumor as assessed by FACS, and exhibited significant T cells penetrating the tumor by IHC, but demonstrated outgrowth of Ag⁻ tumor cells (FIG. 4L and FIGS. 12A-12B).

To uncouple the effects of TRAIL and the CAR, RT-sensitized mice were treated with L(del) CAR T cells, which bind tumor but do not induce CAR cytotoxicity or TRAIL upon recognition, and L(del)-TRAIL CAR T cells, which bind tumor and constitutively express TRAIL but exert no CAR-mediated cytotoxicity. While the first strategy yielded no response despite local T cell accumulation (FIG. 4M), targeting constitutive TRAIL-expressing T cells to the tumor using the external CAR domain modestly increased the response rate (FIG. 4M).

Localized RT Effectively Conditions Tumor for Subsequent CAR T Cell Administration

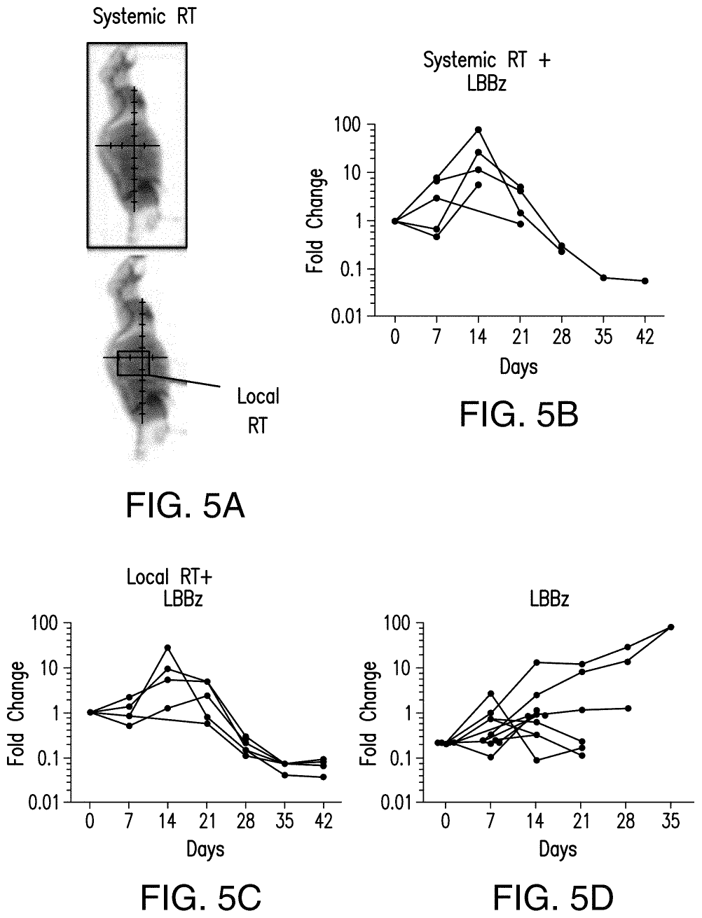

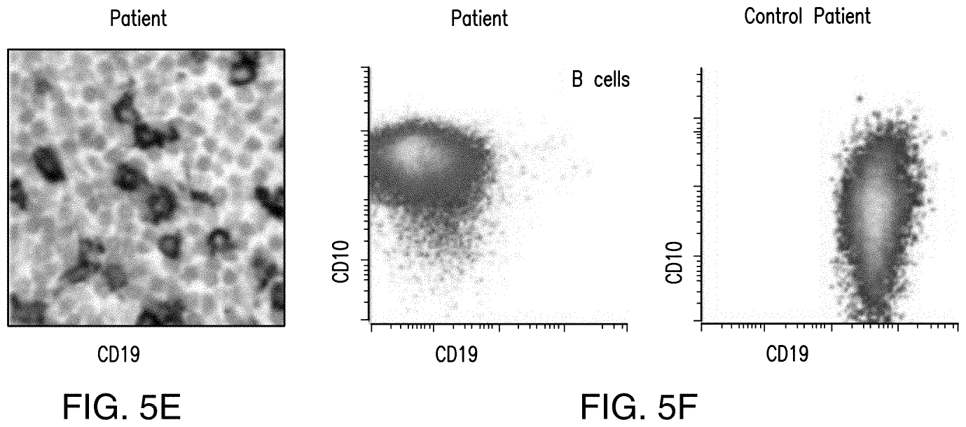

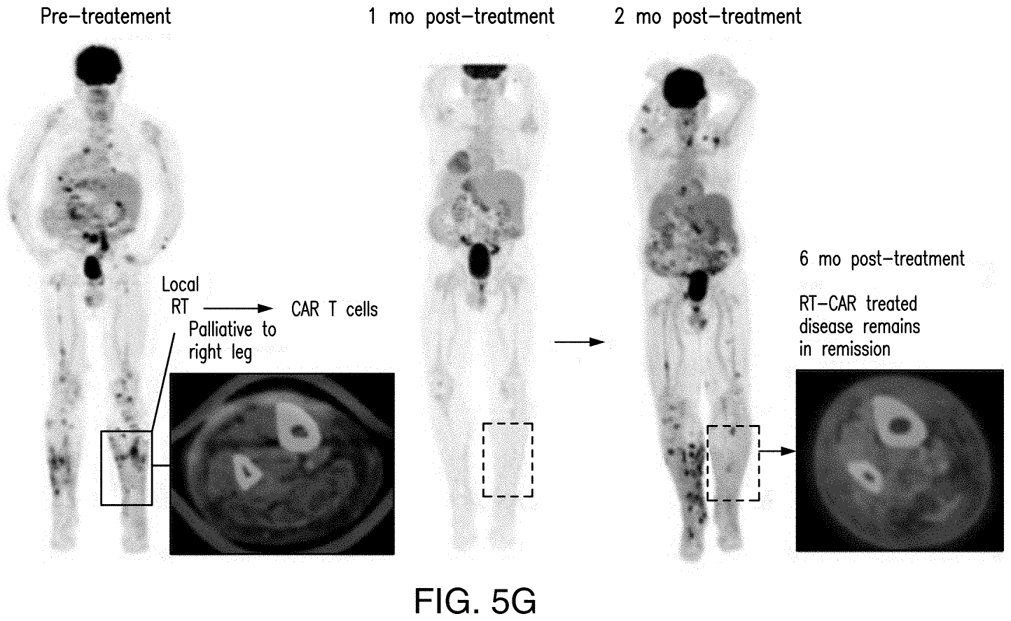

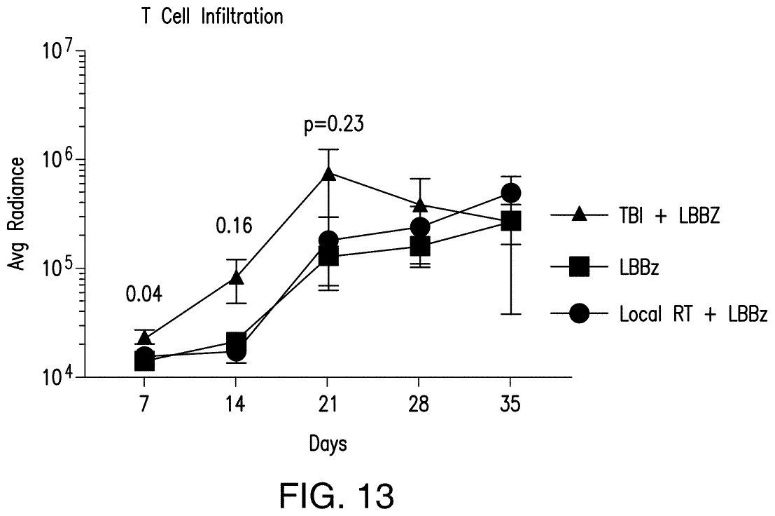

To determine whether systemic RT is required for CAR T cell sensitization, or if local RT to the tumor would suffice, mice harboring orthotopic PDAC were treated with RT to the whole body or only the pancreatic tumor, followed by CAR T cell administration (FIG. 5A). While total body RT-treated mice tended to have greater T cell tumor infiltration at early time points (FIG. 13), both strategies resulted in similar tumor responses (FIGS. 5B-5D). Thus, despite potentially different host effects between systemic and local low-dose RT, either approach effectively sensitizes heterogeneous tumor to CAR T cell killing.

RT and CAR T Cell Treatment in Patient with Heterogeneous Tumor: A Case Report

Experience combining RT with CAR T cells is limited. Just as tumor cells transcriptionally primed for TRAIL-mediated killing by RT exhibited significantly more death in response to CAR T cells in the cell culture and mouse studies, it is conceivable that similar sensitization may occur in nearby antigen-negative normal tissue cells after RT. A patient with refractory diffuse large B cell lymphoma (DLBCL) bearing a large proportion of CD19⁻ tumor cells in the sampled tumor masses (FIGS. 5E-5F) presented for CD19 CAR therapy (NCT02631044). The patient had painful disease infiltrating the skin of his lower legs, particularly on the right. Palliative RT was offered to his right leg (4 Gy×5 fractions), and the patient then received CD19 CAR T cells as planned. In the days and weeks post CAR T cell therapy, the patient did not exhibit signs or symptoms of toxicity within the irradiated field. The patient exhibited a grade 2 CRS, without neurological symptoms. One month post-CAR T cells, the patient had an excellent response by PET-CT imaging (FIG. 5G). Two months post-CAR T cell infusion, tumor rebounded in prior and new locations with CD19-low/negative expression, with the exception of the diseased area that received palliative RT before CAR T cells. Currently, one year after treatment, the area of antigen-heterogeneous tumor subjected to palliative RT followed by CAR T cells remains in CR (FIG. 5G).

Discussion

The initial choice to target CD19 in B cell malignancies was largely driven by the elevated and relatively homogeneous expression of CD19 in leukemia and lymphoma and its confinement to the B cell lineage in normal tissues (Brentjens et al., Nat Med (2003); 9:279-286; Maher et al., Nat Biotechnol (2002); 20:70-75). Based on the remarkable complete remission rates of 70-90% in phase I ALL trial patients (Sadelain, J Clin Invest (2015); 125:3392-3400), the prospect of extending CAR therapy to a wide range of cancers is intriguing. While CAR therapy has only recently begun to tackle solid tumors (Zhao et al., Cancer Cell (2015); 28:415-428; Morello et al., Cancer Discov (2016); 6:133-146; Jindal et al., Med Oncol (2018); 35:87), results have so far been modest with few occurrences of major responses (Louis et al., Blood (2011); 118:6050-6056; Brown et al., N Engl J Med (2016); 375:2561-2569). Escape and regrowth of antigen-negative tumor cells now being a well-documented mechanism of resistance to CAR therapy (Brown et al., N Engl J Med (2016); 375:2561-2569; Gardner et al., Blood (2016); 127:2406-2410; Jackson and Brentjiens, Cancer Discov (2015); 5:1238-1240), novel approaches are needed to enable CART cells to effectively prevent antigen escape.

An early approach to overcoming antigen escape from CAR T cells is to target two different antigens (Hegde et al., J Clin Invest (2016); 126:3036-3052). Another makes use of “armored CARs” to recruit endogenous T cells via the secretion of activating cytokines such as IL-18 (Avanzi et al., Cell Rep (2018); 23: 2130-2141) or the expression of costimulatory ligands (Zhao et al., Cancer Cell (2015); 28:415-428). Checkpoint inhibitor therapy has since been added to CAR T cells, aiming to reinvigorate both the CAR T cells and endogenous tumor-reactive T cells (Suarez et al., Oncotarget (2016); 7:34341-34355; Cherkassky et al., J Clin Invest (2016); 126:3130-3144). However, all of these approaches rely upon tumor cells to express a tumor-specific antigen that is recognized by either CARs or TCRs. None of these approaches provide a mechanism by which a tumor cells lacking both CAR targets and immunogenic TCR epitopes could be eliminated by T cells.

The approach reported here delineates a mechanism by which a tumor cell still be eliminated by CAR T cells in trans, irrespective of immunogenicity. This approach is thus relevant to preempt antigen escape and may be particularly beneficial in tumors with low mutational burden, where the probability of neoantigen presentation and recognition is low.

The spatial and temporal specificity achieved here relies upon a physiologic response of the CAR T cell and radiosensitization of tumor cells irrespective of target expression. The observation that TRAIL is induced in CAR T cells after tumor encounter ensures active and maximal production within the tumor microenvironment. The ability to induce TRAIL receptor on Ag⁺ and Ag⁻ tumor cells through targeted RT provides a window of opportunity to enhance site-specific CAR T cell efficacy against heterogeneous tumor. The effect of this interaction has multiple implications. It was found that both systemic and localized RT sensitize the tumor to CAR T cell killing. Most importantly, in antigen-heterogeneous pancreatic cancer, it was shown that Ag⁻ tumor cells that would otherwise escape CAR recognition can be eliminated by CAR T cells after low dose RT in vivo. In the case of systemic disease, low dose total body irradiation may effectively sensitize tumor cells and results in elimination at lower CAR T cell dose, potentially reducing the risk for cytokine release syndrome while increasing the efficacy.

The early observation that tumor cells are highly sensitive to TRAIL-induced apoptosis relative to normal cells (Walczak et al., Nat Med (1999); 5:157-163) generated enthusiasm for recombinant TRAIL or agonistic TRAIL receptor-based therapies. Unfortunately this therapy has encountered multiple limitations, including short half-life of TRAIL protein (Ichikawa et al., Nat Med (2001); 7: 954-960), reduced apoptotic ability of bivalent antibody (Waj ant, Cell Death Differ (2015); 22:1727-1741), limited local tumor penetration when administered systemically, and downstream resistance to apoptosis through tumor gene expression changes (Ichikawa et al., Nat Med (2001); 7: 954-960). CART cells as the source of TRAIL offer several potential advantages, such as concentrated TRAIL synthesis within the tumor, continuous production as long as tumor and T cells are present, and supply of native trimeric protein rather than potentially less apoptotic bivalent antibody (Wajant, Cell Death Differ (2015); 22:1727-1741). Although TRAIL may exert a pro-apoptotic effect on CAR T cells through death receptor 5 (Tschumi et al., J Immunother Cancer (2018); 6:71), this activity is not increased by radiation conditioning prior to CAR T cell infusion.

Several other forms of immunotherapy are commonly combined with RT under certain circumstances. An “immunogenic,” ablative high dose of radiation induces tumor death and in some contexts leads to increased antigen presentation, subsequent T cell activation, and potentially an “abscopal” or secondary immune response against unirradiated tumor (Spiotto et al., Sci Immunol (2016); 1). Due to the infrequency of the abscopal effect in clinical practice, predictably harnessing this phenomenon remains an active area of investigation. Unlike endogenous T cells, CAR T cells do not rely on antigen presentation, and unless radiation induces expression of the particular CAR target molecule (Weiss et al., Cancer Res (2018); 78:1031-1043), it is not intuitive whether radiation may have an immunogenic, immunosuppressive, or irrelevant effect on CAR T cell therapy. A fundamentally different type of “immunogenic radiation” in the context of CAR T cell therapy was described: one by which a sublethal, low dose of radiation locally sensitizes tumor to CAR T cell killing in trans. Unlike its ablative counterpart, sensitizing radiation is not limited by location or size of disease, and given the much lower dose may be applied to wider areas for patients with diffuse metastases, with less concern for RT-related side effects.

A patient with heterogeneous tumor treated with palliative (non-curative) RT before CAR T cell therapy exhibited results consistent with the mouse data, without signs of excess toxicity. Although this clinical correlate aligns with the animal findings, it does not test the hypothesis. In particular, the effect of RT alone on the lasting complete response of his heterogeneous tumor cannot be ignored. However, the administered radiation dose was unable to eliminate his tumor ex vivo, consistent with it being roughly half the standard locally curative dose of >45 Gy for gross disease in this type of aggressive lymphoma (Ng et al., International journal of radiation oncology, biology, physics (2018); 100:652-669). Further, although toxicity was not observed in the RT field of the leg, it is possible other normal tissues, such as the GI system, may exhibit heightened RT sensitivity to activated CAR T cell produced TRAIL (Finnberg et al., Cancer Res (2016); 76:700-712). A clinical trial incorporating RT with CAR T cells is planned to assess the effect on clonal antigen heterogeneity, the safety of RT conditioning, and systemic effects of local RT on CAR T cell-mediated disease response.

RT is currently used at some point in the treatment of about half of metastatic cancer patients for palliation, and is commonly utilized in almost all non-metastatic cancer types as an alternative, or an addition to surgery to increase local control (Miller et al., CA Cancer J Clin (2016); 66:271-289).

Implementing CAR therapy within current RT regimens may further increase local and systemic tumor control. The findings suggest that integrated delivery of these two therapies warrants coordination among disease management teams.

The findings support the concept that multimodality CAR therapy with RT conditioning may improve responses in solid tumors. Most importantly, a mechanistic platform by which engineered T cells can be further enhanced to eliminate clonally heterogeneous solid tumors was provided.

Materials and Methods

Cell Culture

Tumor cells expressing firefly luciferase-GFP were described previously (Zhao et al., Cancer Cell (2015); 28:415-428). 293T cell line, H29 and retroviral packaging cell lines were cultured in DMEM supplemented with 10% FCS (Zhao et al., Cancer Cell (2015); 28:415-428). Capan-2 cell were generously provided by Jason S. Lewis (MSKCC) and grown in RPMI supplemented with 10% FCS. Cells were tested for mycoplasma using the MycoAlert Mycoplasma Detection Kit (Lonza) prior to injection into the animals.

Buffy coats from healthy volunteer donors were obtained from the New York Blood Center. Peripheral blood mononuclear cells were isolated by density gradient centrifugation, and cells were then stimulated with PHA (Sigma) and cultured as previously described (Zhao et al., Cancer Cell (2015); 28:415-428).

Radiation

Radiation dose: All experiments using PDAC used 2 Gy, unless otherwise specified. For in vitro RT studies, all RT sensitization experiments were performed with RT given to the tumor cells two days prior to tumor analysis or coculture with T cells, unless specified otherwise.

Radiation method: Local RT to the pancreas was performed by identifying the pancreatic tumor using intraperitoneal contrast and cone beam CT imaging on an X-Rad 225Cx machine, which combines high-accuracy cone beam CT imaging with 3D image guided radiation treatment under general anesthesia. Local RT was delivered using either anterior-posterior, or anterior-posterior and lateral beams. Experiments requiring less target precision (total body RT) were performed using the small animal irradiator with open jaws in the AP direction.

Flow Cytometry

Fluorochrome-conjugated antibodies to CD3 (UCHT1), CD4 (S3.5), CD8 (3B5), DR5 (DJR2-4, PE-conjugated, BioLegend), CD95 (DX2, PE-Cy7-conjugated, BD Biosciences), LeA (7LE, AF405-conjuugated, Novus), CD19 (SJ25C1), 41BBL (5F4), and Granzyme B (FGB12, Invitrogen) were used. Alexa 647-conjugated goat anti-human F(ab)2 (ThermoFisher) was used to detect CARs. Flow cytometry was performed on a BD LSRII and data analyzed with FlowJo software Ver. 9.5.2 (TreeStar). Fc Receptor Binding Inhibitor Antibody Human (eBioscience) was used to block Fc receptors. In some cases, CountBright beads (Invitrogen) were added to samples to count cell numbers.

TRAIL Measurements

For RNA and ELISA experiments, CAR T cells were exposed Capan2 expressing the target antigen for 4 hours followed by removal and monoculture of T cells, which were replated in new media daily. Cells were removed and analyzed for TRAIL mRNA expression at given time points, and media was collected at the end of each day for TRAIL ELISA (MyBiosource MBS335491). qPCR was performed using the TaqMan system (ThermoFisher), using primers Hs00921974 (TRAIL), Hs00366278 (DR5), and Hs04194366 (RPL13A housekeeping).

RNA Extraction and Real-Time Quantitative PCR

Total RNA was extracted from cells by using the RNeasy kit (QIAGEN), following the manufacturer's instructions. RNA concentration and quality were assessed by UV spectroscopy using the NanoDrop spectrophotometer (Themo Fisher Scientific). One hundred to 200 ng total RNA were used to prepare cDNA using the SuperScript III First-Strand Synthesis SuperMix (Invitrogen), with a 1:1 volume ratio of random hexamers and oligo dT. Completed cDNA synthesis reactions were treated with 2U RNase H for 20 min at 37° C. Quantitative PCR was performed using the ABsolute Blue qPCR SYBR Green Low ROX Mix. PCR assays were run on the QuantStudio™ 7 Flex System, and Cₜ values were obtained with the QuantStudio Real-Time PCR software. Relative changes in gene expression were analysed with the 2^{ΔCt} method.

Vector Constructs

The 1928 and 19BBζ CARs, which comprise the SJ25C1 CD19-specific scFv have been previously described (Maher et al., Nat Biotechnol (2002); 20:70-75). LBBz and L28z were constructed by replacing the CD19-specific scFv with the human 5B1 scFv targeting Le^{A}. All constructs were designed to express Gaussia Luciferase for T cell imaging as previously described (Santos et al., Nat Med (2009); 15:338-344). L(del) mutants were created by removing the intracellular costimulatory and signaling domains from the specified construct, while retaining the extracellular and transmembrane portion. Constructs expressing TRAIL were created by adding the TRAIL cDNA sequence following the specified CAR and a P2A sequence.

Retroviral Production and Transduction

Plasmids encoding the SFG γ-retroviral (RV) vector (Riviere et al., Proc Natl Acad Sci USA (1995); 92:6733-6737) were prepared as previously described (Maher et al., Nat Biotechnol (2002); 20:70-75). VSV-G pseudotyped retroviral supernatants derived from transduced gpg29 fibroblasts (H29) were used to construct stable retroviral-producing cell lines as previously described (Gallardo et al., Blood (1997); 90:952957). T cells were transduced by centrifugation on Retronectin (Takara)-coated plates. In T cell knockout studies, CAR transduction was performed directly after Cas9/gRNA electroporation as described (Eyquem et al., Nature (2017); 543, 113-117).

Cytotoxic T Lymphocyte Assays (CTLs)

CTLs using 100% Ag+ tumor cells: The cytotoxicity of T cells transduced with a CAR was determined by standard luciferase based assays. For luciferase based assays, tumor cells expressing firefly luciferase-GFP served as target cells. The effector and tumor cells were co-cultured at indicated E/T ration in the black-walled 96 or 384 well plates in triplicate manner. Target cells alone were plated at the same cell density to determine the baseline luciferase expression (no T cell control). 18 hr later, luciferase substrate (Bright-Glo, Promega) was directly added to each well. Emitted light was measured by luminescence plate reader or Xenogen IVIS Imaging System (Xenogen) with Living Image software (Xenogen) for acquisition of imaging data sets. Lysis was determined as [1−(RLUsample)/(RLUmax)]×100. Assays were performed using CAR T cells transduced within the previous week.

CTLs using 75% Ag+, 25% Ag− tumor cells: In experiments involving pre-stimulated CAR T cells, all CAR T cells were grown for 10-12 days at a constant concentration of 1 million cells/ml in the presence of 20U/ml IL-2, reconstituted every other day. Cells were stimulated before CTL by adding the CAR T cells to adherent cells containing the target antigen (Le^{A}+ Capan2) 1 day prior to the experiment, and removing the T cells by aspiration the day of the experiment. CAR T cells were then cocultured with RT-sensitized 75% Ag⁺ Capan2 PDAC at an E:T ratio of 1:3 in 48 well plates. Percent killing relative to no treatment controls, in addition to the relative number of Ag+ and Ag− tumor cells remaining, was determined at the pre-specified time points of 4 days for LBBz CAR T cell cultures, and 5 days for L(del) CAR T cell cultures. In experiments that specifically quantified Ag− cell killing, only Ag− cells expressed Luciferase.

In all cytotoxicity assays where RT was used, tumor cells (Capan2) were given RT, grown for two days in culture, then live cells were incubated with CAR T cells.

Video Microscopy

Ag⁻ cells labeled with CTV (CellTrace Violet, Fisher C34571) were mixed with unlabeled Ag⁺ cells at a ratio of 75% LeA⁺, in addition to CAR T cells in 8-well microscopy slides, and Annexin-V 595 (Fisher A13203). Confocal images were acquired every 7 minutes over 18 hours in culture at optimal imaging parameters with a LSM 880 Confocal Microscope (Carl Zeiss). Data was 3D-rendered and visualized using Imaris (Bitplane). Percent killing of LeA⁻ cells was determined at every time point using a custom macro made in Image)/FIJI (NIH), which automatically quantified total Ag⁻ cells (blue cells) and dead/dying Ag⁻ cells (double red and blue positive).

Gene Disruption

48 h after initiating T-cell activation, the cells were transfected by electrontransfer of Cas9 mRNA and gRNA using an AgilePulse MAX system (Harvard Apparatus). 3×10⁶ cells were mixed with 5 μg of Cas9 and 5 μg of gRNA into a 0.2 cm cuvette. Following electroporation cells were diluted into culture medium and incubated at 37° C., 5% CO₂. To obtain TCR-negative T cells, TCR-positive T cells were removed 3-5 days after gRNA transfection using magnetic biotin-anti-TCRαβ and anti-biotin microbeads and LS columns (Miltenyi Biotech). To obtain TRAIL-negative cells, TRAIL-positive T cells were removed using magnetic PE-anti-TRAIL (R&D, FAB687P) and anti-PE microbeads in LS columns (Miltenyi Biotech). To obtain DR5-negative cells, FACS sorting was performed using PE-anti-DR5 staining.

For TCR knockout, a gRNA that targets a sequence in the first exon of the constant chain of the TCRα gene (TRAC) that is required for the TCRα and β assembly and addressing to the cell-surface was used, as previously described⁴². TRAIL was performed using synthetic modified gRNA kits (Synthego). Guide RNAs were reconstituted at 1 μg μl⁻¹ in cytoporation T Buffer (Harvard Apparatus). Cas9 mRNA was synthesized by TriLink Biotechnologies.

Pancreatic Cancer Tumor Model

8- to 12-week-old NOD/SCID/IL-2Ry-null (NSG) male mice (Jackson Laboratory) were used, under a protocol approved by the MSKCC Institutional Animal Care and Use Committee. Specified ratios of LeA+ and LeA− FACS-sorted Capan2 PDAC tumor cells were injected into the pancreas of NSG mice after surgically opening the mice and exposing the pancreas under IRB-approved mouse protocol. 75,000 tumor cells were injected per mouse, in 50% matrigel. Mice were randomized to treatment and treatment groups were blinded to personnel performing treatment and tumor assessment. Tumor established in the pancreas for 9 days, then mice were treated with RT followed by T cells. Tumor volume was measured by Bioluminescence Imaging (BLI) using retro-orbital D-luciferin injection followed by IVIS imaging. Tumor burden for each mouse was expressed over time relative to that mouse's baseline tumor BLI at the beginning of treatment.

T Cell Imaging

CAR T cells containing Gaussia Luciferase were imaged using coelenterazine (3031-10 Coelenterazine-SOL in vivo, Nanolight), injected retro-orbitally.

Transcriptome Analysis

Cells were lysed in Trizol LS (Invitrogen) and then submitted to the Integrated Genomics Operation at MSKCC for RNA extraction. After ribogreen quantification and quality control on bioAnalyser, 500 ng of total RNA underwent library preparation using the Truseq Stranded Total RNA library preparation chemistry (Illumina), with 6 cycles of PCR. Samples were barcoded and run on a Hiseq 2500 1T in a 50 bp/50 bp Paired end run, using the TruSeq SBS Kit v3 (Illumina). An average of 51 million paired reads were generated per sample and the percent of mRNA bases was 58% on average.

The output FASTQ data files were mapped to the target genome using the rnaStar aligner which maps reads genomically and resolves reads across splice junctions. The 2 pass mapping method was used in which the reads are mapped twice. The first mapping pass uses a list of known annotated junctions from Ensemble. Novel junctions found in the first pass are then added to the known junctions and a second mapping pass is done (on the second pass the RemoveNoncanoncial flag is used). After mapping, the output SAM files were post processed using the PICARD tools to: add read groups, AddOrReplaceReadGroups which in additional sorts the file and coverts it to the compressed BAM format. The expression count matrix from the mapped reads were computed using HTSeq (www-huber.embl.de) and one of several possible gene model databases. The raw count matrix generated by HTSeq are then be processed using the R/Bioconductor package DESeq (www-huber.embl.de) which is used to both normalize the full dataset and analyze differential expression between sample groups.

For GSA the Bioconductor package PIANO was used (bioconductor.org). The precise call is: gsa.res <-runGSA(fc,geneSetStat=“mean”,gsc=gsc, gsSizeLim=c(min.gns,max.gns),nPerm=nPerm), where fc==foldChange, min.gns==5, max.gns==1000, nPerm==1e4. For GeneSets the MSigDb from the Broad was used (software.broadinstitute.org). The following collections were used: “c1.all.v4.0.symbols.gmt”, “c2.all.v4.0.symbols.gmt”, “c3.all.v4.0.symbols.gmt”, “c5-1.all.v4.0.symbols.gmt”, “c6-1.all.v4.0.symbols.gmt”, “c7.all.v4.0.symbols.gmt”.

Statistics

All experimental data are presented as mean±s.e.m. No statistical methods were used to predetermine sample size. Groups were compared using unpaired, two-tailed t-test. Statistical analysis was performed on GraphPad Prism 7 software.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A chimeric antigen receptor (CAR), comprising an extracellular antigen-binding domain, a transmembrane domain, and an intracellular domain, wherein the extracellular antigen-binding domain specifically binds to Sialyl Lewis A, and comprises: a heavy chain variable region and a light chain variable region, wherein the heavy chain variable region comprises a CDR3 comprising the amino acid sequence set forth in SEQ ID NO: 3 or a conservative modification thereof, and the light chain variable region comprises a CDR3 comprising the amino acid sequence set forth in SEQ ID NO: 6 or a conservative modification thereof.

2. The CAR of claim 1, wherein the heavy chain variable region comprises a CDR2 comprising the amino acid sequence set forth in SEQ ID NO: 2 or a conservative modification thereof, and the light chain variable region comprises a CDR2 comprising the amino acid sequence set forth in SEQ ID NO: 5 or a conservative modification thereof.

3. The CAR of claim 1, wherein the heavy chain variable region comprises a CDR1 comprising the amino acid sequence set forth in SEQ ID NO: 1 or a conservative modification thereof, and the light chain variable region comprises a CDR1 comprising the amino acid sequence set forth in SEQ ID NO: 4 or a conservative modification thereof.

4. The CAR of claim 1, wherein the heavy chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and/or the light chain variable region comprises a CDR1 comprising the amino acid sequence set forth in SEQ ID NO: 4, a CDR2 comprising the amino acid sequence set forth in SEQ ID NO: 5, and a light chain variable region CDR3 comprising the amino acid sequence set forth in SEQ ID NO: 6.

5. The CAR of claim 1, wherein the heavy chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the light chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

6. The CAR of claim 1, wherein the heavy chain variable region comprises an amino acid sequence that is at least about 80% homologous or identical to the amino acid sequence set forth in SEQ ID NO: 7, and/or the light chain variable region comprises an amino acid sequence that is at least about 80% homologous or identical to the amino acid sequence set forth in SEQ ID NO: 8.

7. The CAR of claim 6, wherein the heavy chain variable region comprises the amino acid sequence set forth in SEQ ID NO: 7, and the light chain variable region comprises the amino acid sequence set forth in SEQ ID NO: 8.

8. The CAR of claim 1, wherein the extracellular antigen-binding domain comprises a single-chain variable fragment (scFv), a Fab, or a F(ab)₂.

9. The CAR of claim 8, wherein the scFv is a human scFv.

10. The CAR of claim 8, one or more of the scFv, Fab and F(ab)₂ are comprised in a fusion protein with a heterologous sequence to form the extracellular antigen-binding domain.

11. The CAR of claim 1, wherein the extracellular antigen-binding domain comprises a linker between the heavy chain variable region and the light chain variable region.

12. The CAR of claim 1, wherein a signal peptide is covalently joined to the 5′ terminus of the extracellular antigen-binding domain.

13. The CAR of claim 1, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide a PD-1 polypeptide, a LAG-3 polypeptide, a 2B4 polypeptide, a BTLA polypeptide, or a combination thereof.

14. The CAR of claim 1, wherein intracellular domain comprises a CD3ζ polypeptide.

15. The CAR of claim 14, wherein the intracellular domain further comprises at least one co-stimulatory signaling region.

16. The CAR of claim 15, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

17. The CAR of claim 16, wherein the at least one co-stimulatory signaling region comprises a CD28 polypeptide.

18. The CAR of claim 14, wherein the CD3ζ polypeptide is a wild-type CD3ζ polypeptide or a modified CD3ζ polypeptide, wherein the modified CD3ζ polypeptide lacks a) all or part of immunoreceptor tyrosine-based activation motifs (ITAMs), wherein the ITAMs are ITAM1, ITAM2, and ITAM3; and/or lacks all or part of basic-rich stretch (BRS) regions, wherein the BRS regions are BRS1, BRS2, and BRS3.

19. The CAR of claim 18, wherein the modified CD3ζ polypeptide:
a) lacks ITAM2 or a portion thereof, optionally further lacks i) ITAM3 or a portion thereof, and/or ii) ITAM1 or a portion thereof;
b) lacks ITAM1 or a portion thereof, optionally further lacks ITAM3 or a portion thereof;
c) lacks ITAM3 or a portion thereof;
d) comprises a deletion of ITAM2 or a portion thereof, optionally further comprises i) a deletion of ITAM3 or a portion thereof, and/or ii) a deletion of ITAM1 or a portion thereof;
e) comprises a deletion of ITAM1 or a portion thereof, optionally further comprises a deletion of ITAM3 or a portion thereof; and/or
f) comprises a deletion of ITAM3 or a portion thereof.

20. The CAR of claim 18, wherein the modified CD3ζ polypeptide:
a) lacks BRS2 or a portion thereof, and optionally further lacks i) BRS3 or a portion thereof, and/or ii) BRS1 or a portion thereof;
b) lacks BRS1 or a portion thereof, and optionally further lacks BRS3 or a portion thereof;
c) lacks BRS3 or a portion thereof; and/or
d) lacks BRS1 or portion thereof, BRS2 or portion thereof, and BRS3 or a portion thereof;
e) comprises a deletion of BRS2 or a portion thereof, and optionally further comprises i) a deletion of BRS3 or a portion thereof, and/or ii) a deletion of BRS1 or a portion thereof;
f) comprises a deletion of BRS1 or a portion thereof, and optionally further comprises a deletion of BRS3 or a portion thereof;
g) comprises a deletion of BRS3 or a portion thereof; and/or
h) comprises a deletion of BRS1 or portion thereof, BRS2 or portion thereof, and BRS3 or a portion thereof.

21. The CAR of claim 18, wherein the modified CD3ζ polypeptide lacks ITAM2, ITAM3, BRS2, and BRS3, or comprises a deletion of ITAM2, ITAM3, BRS2, and BRS3.

22. The CAR of claim 1, further comprising a hinge/spacer region.

23. The CAR of claim 22, wherein the hinge/spacer region is a native or modified hinge/spacer region of a molecule selected from the group consisting of a CD8 polypeptide, a CD28 polypeptide, a CD3ζ polypeptide, a CD4 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide or a combination thereof.

24. The CAR of claim 22, wherein:
a) the hinge/spacer region comprises a hinge spacer region of a CD28 polypeptide and the transmembrane domain comprises a transmembrane domain of a CD28 polypeptide;
b) the hinge/spacer region comprises a hinge spacer region of a CD84 polypeptide and the transmembrane domain comprises a transmembrane domain of a CD84 polypeptide;
c) the hinge/spacer region comprises a hinge spacer region of a CD166 polypeptide and the transmembrane domain comprises a transmembrane domain of a CD166 polypeptide;
d) the hinge/spacer region comprises a hinge spacer region of a CD8a polypeptide and the transmembrane domain comprises a transmembrane domain of a CD8a polypeptide; or
e) the hinge/spacer region comprises a hinge spacer region of a CD8b polypeptide and the transmembrane domain comprises a transmembrane domain of a CD8b polypeptide; or
f) the hinge/spacer region comprises a hinge spacer region of a CD28 polypeptide and the transmembrane domain comprises a transmembrane domain of a ICOS polypeptide.

25. The CAR of claim 1, wherein the CAR is recombinantly expressed, or expressed from a vector.

26. An immunoresponsive cell comprising the CAR of claim 1.

27. The immunoresponsive cell of claim 26, wherein the immunoresponsive cell is selected from the group consisting of a T cell, a Natural Killer (NK) cell, a lymphoid progenitor cell, a T cell-precursor cell, and a pluripotent stem cell from which a lymphoid cell may be differentiated.

28. The immunoresponsive cell of claim 27, wherein the immunoresponsive cell is a T cell.

29. The immunoresponsive cell of claim 28, wherein the T cell is selected from the group consisting of a cytotoxic T lymphocyte (CTL), a regulatory T cell, and central memory T cells.

30. A nucleic acid molecule comprising a nucleic sequence encoding the chimeric antigen receptor (CAR) of claim 1.

31. A vector comprising the nucleic acid molecule of claim 30.

32. A host cell comprising the vector of claim 31.

33. A method for producing an immunoresponsive cell that binds to Sialyl Lewis A, comprising introducing into the immunoresponsive cell a nucleic acid molecule comprising a nucleic acid sequence that encodes the CAR of claim 1.

34. A composition comprising the immunoresponsive cell of claim 1.

35. A method of treating or preventing a malignant growth in a subject, comprising administering to the subject an effective amount of the immunoresponsive cell of claim 1.

36. A kit for treating or preventing a malignant growth, comprising the immunoresponsive cell of claim 1.
